(19) United States
(12) Patent Application Publication
(10) Pub. No.: US 2008/0025966 A1
(43) Pub. Date: Jan. 31, 2008

(54) Methods And Compositions For The Treatment Of Gastrointestinal disorders

(76) Inventor: Mark G. Currie, Sterling, MA (US)

Correspondence Address: FISH & RICHARDSON PC, P.O. BOX 1022, MINNEAPOLIS, MN, 55440-1022, US

(21) Appl. No.: 10/587,450

(22) PCT Filed: Jan. 31, 2005
(86) PCT No.: PCT/US05/02941

§ 371 Date: Apr. 12, 2007

Related U.S. Application Data
(60) Provisional application No. 60/540,675, filed on Jan. 30, 2004.

(51) Int. Cl. A61K 38/48; A61P 1/00; A61P 1/12

(52) U.S. Cl. 424/94.64

(57) ABSTRACT

The use of chymotrypsin inhibitors for treating various disorders, including IBS and other gastrointestinal disorders and conditions (e.g., gastrointestinal motility disorders, functional gastrointestinal disorders, gastroesophageal reflux disease (GERD), Crohn's disease, ulcerative colitis, inflammatory bowel disease, functional heartburn, dyspepsia (including functional dyspepsia or nonulcer dyspepsia), gastroparesis, chronic intestinal pseudo-obstruction (or colonic pseudoobstruction), and disorders and conditions associated with constipation is described.

### TECHNICAL FIELD

This invention relates to methods and compositions for treating gastrointestinal disorders, obesity, congestive heart failure, benign prostatic hyperplasia and other disorders including hypertension and asthma.

### BACKGROUND

Irritable bowel syndrome (IBS) is a common chronic disorder of the intestine that affects 20 to 60 million individuals in the US alone (Lehman Brothers, Global Healthcare-Irritable Bowel Syndrome Industry Update, September 1999). IBS is the most common disorder diagnosed by gastroenterologists (28% of patients examined) and accounts for 12% of visits to primary care physicians (Camilleri 2001 Gastroenterology 120:652-668). In the US, the economic impact of IBS is estimated at $25 billion annually, through direct costs of health care use and indirect costs of absenteeism from work (Talley 1995 Gastroenterology 109:1736-1741). Patients with IBS have three times more absenteeism from work and report a reduced quality of life. Sufferers may be unable or unwilling to attend social events, maintain employment, or travel even short distances (Drossman 1993 Dig Dis Sci 38:1569-1580). There is a tremendous unmet medical need in this population since few prescription options exist to treat IBS.

Patients with IBS suffer from abdominal pain and a disturbed bowel pattern. Three subgroups of IBS patients have been defined based on the predominant bowel habit: constipation-predominant (c-IBS), diarrhea-predominant (d-IBS) or alternating between the two (a-IBS). Estimates of individuals who suffer from c-IBS range from 20-50% of the IBS patients with 30% frequently cited. In contrast to the other two subgroups that have a similar gender ratio, c-IBS is more common in women (ratio of 3:1) (Talley et al. 1995 Am J Epidemiol 142:76-83).

The definition and diagnostic criteria for IBS have been formalized in the “Rome Criteria” (Drossman et al. 1999 Gut 45:Suppl II:1-81), which are well accepted in clinical practice. However, the complexity of symptoms has not been explained by anatomical abnormalities or metabolic changes. This has led to the classification of IBS as a functional GI disorder, which is diagnosed on the basis of the Rome criteria and limited evaluation to exclude organic disease(Ringel et al. 2001 Annu Rev Med 52: 319-338). IBS is considered to be a “biopsychosocial” disorder resulting from a combination of three interacting mechanisms: altered bowel motility, an increased sensitivity of the intestine or colon to pain stimuli (visceral sensitivity) and psychosocial factors (Camilleri 2001 Gastroenterology 120:652-668). Recently, there has been increasing evidence for a role of inflammation in the etiology of IBS. Reports indicate that subsets of IBS patients have small but significant increases in colonic inflammatory and mast cells, increased inducible nitric oxide (NO) and synthase (iNOS) and altered expression of inflammatory cytokines (reviewed by Talley 2000, Medscape Coverage of DDW Week).

Guanylin is an intestinal peptide that stimulates chloride secretion. In humans, guanylin is produced initially as a 115 amino acid protein referred to as preproguanylin. The mature protein, which is believed to be the active form, has 15 amino acids. Guanylin is inactivated by cleavage by the serine protease, chymotrypsin, which is present in the gastrointestinal tract, and by a chymotrypsin-like enzyme that is present in the liver. Guanylin is an agonist of the transmembrane guanylate cyclase (GC-C) receptor. The GC-C receptor is present on the apical plasma membrane of enterocytes in intestinal tract and in other epithelia. Activation of the GC-C receptor by guanylin in the intestine increases cGMP levels. This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are fluid and electrolyte homeostasis, the regulation of epithelial cell proliferation and the induction of apoptosis (Shailubhai 2002 Curr Opin Drug Dis Devel 5:261-268). Other GC-C receptor agonists include uroguanylin, renoguanylin, lymphoguanylin and the E. coli heat stable ST peptide.

Diarrhea is a common complication in HIV patients. It appears that diarrhea is particularly common in patients treated with protease inhibitors (Sherman et al. 2000 Clin Infect Dis 30:908). Prolonged diarrhea impacts quality of life and can contribute to weight loss, malnutrition, immunosuppression, poor drug absorption, non-compliance with therapy and mortality. Oat bran, psyllium, loperamide, calcium carbonate, pancrelipase, and SP-303 have been shown to have some beneficial effect on diarrhea associated with the use of HIV protease inhibitors (Sherman et al., supra). Bode et al. (AIDS 13:2595, 1999) state that the cause of HIV protease inhibitor associated diarrhea is unknown. Bode et al. also report that saquinavir, ritonavir, and nelfinavir, but not indinavir, impair the epithelial barrier in human HT-29/B6 cells.

### SUMMARY

The present invention features compositions and related methods for treating IBS and other gastrointestinal disorders and conditions (e.g., gastrointestinal motility disorders, functional gastrointestinal disorders, gastroesophageal reflux disease (GERD), Crohn's disease, ulcerative colitis, inflammatory bowel disease, functional heartburn, dyspepsia (including functional dyspepsia or nonulcer dyspepsia), gastroparesis, chronic intestinal pseudo-obstruction (or colonic pseudoobstruction), and disorders and conditions associated with constipation, e.g., constipation associated with use of opiate pain killers, post-surgical constipation, and constipation associated with neuropathic disorders as well as other conditions and disorders.

The methods and the compositions in the invention relate to the administration of a compound that inhibits chymotrypsin activity. By interfering with the inactivation of guanylin in the intestinal tract caused by chymotrypsin cleavage of guanylin, a chymotrypsin inhibitor can potentiate the action of naturally-occurring guanylin, thereby increasing or regularizing intestinal motility. The invention also features the use of a chymotrypsin inhibitor in a combination therapy with therapeutically administered GC-C receptor agonist or some other treatment for a gastrointestinal disorder. In certain embodiments, the GC-C receptor agonist is guanylin or a biologically active variant or fragment thereof.

The present invention also features compositions and related methods for treating obesity, congestive heart failure and benign prostatic hyperplasia (BPH).

Without being bound by any particular theory, in the case of IBS and other gastrointestinal disorders the compositions of the invention are useful because they can increase or regularize gastrointestinal motility by potentiating guanylin activity.

Without being bound by any particular theory, in the case of IBS and other gastrointestinal disorders the compositions of the invention are useful, in part, because they can decrease inflammation by potentiating guanylin activity.

Without being bound by any particular theory, in the case of IBS and other gastrointestinal disorders the compositions of the invention are also useful because they can decrease gastrointestinal pain or visceral pain by potentiating guanylin activity.

The invention features pharmaceutical compositions comprising certain compounds that are capable of reducing the activity of chymotrypsin, particularly the ability of chymotrypsin to inactivate guanylin by proteolytic cleavage. Also within the invention are pharmaceutical compositions comprising a chymotrypsin inhibitor as well as combination compositions comprising a chymotrypsin inhibitor and a second therapeutic agent.

The invention includes methods for treating other disorders such as congestive heart failure and benign prostatic hyperplasia by administering a chymotrypsin inhibitor. Such agents can be used in combination with natriuretic peptides (e.g., atrial natriuretic peptide, brain natriuretic peptide or C-type natriuretic peptide), a diuretic, or an inhibitor of angiotensin converting enzyme.

The invention features methods and compositions for increasing intestinal motility by potentiating the action of guanylin. Intestinal motility involves spontaneous coordinated distentions and contractions of the stomach, intestines, colon and rectum to move food through the gastrointestinal tract during the digestive process.

The invention features a therapeutic or prophylactic method comprising administering a composition comprising an inhibitor of chymotrypsin. For the treatment of gastrointestinal disorders, the inhibitor can be administered orally, by rectal suppository or parenterally.

In various embodiments, the patient is suffering from a gastrointestinal disorder; the patient is suffering from a disorder selected from the group consisting of: a gastrointestinal motility disorder, irritable bowel syndrome, a functional gastrointestinal disorder, gastroesophageal reflux disease, functional heartburn, dyspepsia, functional dyspepsia, nonulcer dyspepsia, gastroparesis, chronic intestinal pseudo-obstruction, colonic pseudo-obstruction, obesity, congestive heart failure, or benign prostatic hyperplasia. In another aspect, the invention features a method for treating a patient suffering from constipation, the method comprising administering a composition comprising, consisting essentially of or consisting of a chymotrypsin inhibitor and a pharmaceutically acceptable carrier. Clinically accepted criteria that define constipation range from the frequency of bowel movements, the consistency of feces and the ease of bowel movement. One common definition of constipation is less than three bowel movements per week. Other definitions include abnormally hard stools or defecation that requires excessive straining (Schiller 2001 Aliment Pharmacol Ther 15:749-763). Constipation may be idiopathic (functional constipation or slow transit constipation) or secondary to other causes including neurologic, metabolic or endocrine disorders. These disorders include diabetes mellitus, hypothyroidism, hyperthyroidism, hypocalcaemia, Multiple sclerosis, Parkinson's disease, spinal cord lesions, Neurofibromatosis, autonomic neuropathy, Chagas disease, Hirschsprung disease and cystic fibrosis. Constipation may also be the result of surgery or due to the use of drugs such as analgesics (like opioids), antihypertensives, anticonvulsants, antidepressants, antispasmodics and antipsychotics.

In various embodiments, the constipation is associated with use of a therapeutic agent; the constipation is associated with a neuropathic disorder; the constipation is post-surgical constipation (postoperative ileus); the constipation is associated with a gastrointestinal disorder; the constipation is idiopathic (functional constipation or slow transit constipation); the constipation is associated with neuropathic, metabolic or endocrine disorder (e.g., diabetes mellitus, hypothyroidism, hyperthyroidism, hypocalcaemia, Multiple Sclerosis, Parkinson's disease, spinal cord lesions, neurofibromatosis, autonomic neuropathy, Chagas disease, Hirschsprung disease or cystic fibrosis). Constipation may also be the result of surgery or due to the use of drugs such as analgesics (e.g., opioids), antihypertensives, anticonvulsants, antidepressants, antispasmodics and antipsychotics.

In another aspect, the invention features a method for treating a patient suffering from a gastrointestinal disorder, the method comprising administering a composition comprising, consisting essentially of or consisting of a chymotrypsin inhibitor and a pharmaceutically acceptable carrier.

In various embodiments, the patient is suffering from a gastrointestinal disorder; the patient is suffering from a disorder selected from the group consisting of: a gastrointestinal motility disorder, irritable bowel syndrome, chronic constipation, post-operative ileus, a functional gastrointestinal disorder, gastroesophageal reflux disease, functional heartburn, dyspepsia, functional dyspepsia, nonulcer dyspepsia, gastroparesis, chronic intestinal pseudo-obstruction, Crohn's disease, ulcerative colitis, Inflammatory bowel disease, colonic pseudo-obstruction, obesity, congestive heart failure, or benign prostatic hyperplasia.

In another aspect, the invention features a method for increasing gastrointestinal motility in a patient, the method comprising administering to the patient a composition comprising, consisting essentially of or consisting of a chymotrypsin inhibitor and a pharmaceutically acceptable carrier In another aspect, the invention features a method for decreasing gastrointestinal pain or visceral pain in a patient, the method comprising administering to the patient a composition comprising, consisting essentially of or consisting of a chymotrypsin inhibitor and a pharmaceutically acceptable carrier composition comprising a chymotrypsin inhibitor.

In another aspect, the invention features a method for increasing the activity of an intestinal guanylate cyclase (GC-C) receptor in a patient, the method comprising administering to the patient a composition comprising, consisting essentially of or consisting of a chymotrypsin inhibitor and a pharmaceutically acceptable carrier.

In another aspect, the invention features a method for increasing the level of cGMP in a patient, the method comprising administering to the patient a composition comprising, consisting essentially of or consisting of a chymotrypsin inhibitor and a pharmaceutically acceptable carrier.

In another aspect, the invention features a method for treating a disorder ameliorated by increasing cGMP levels, the method comprising, consisting essentially of or consisting of a chymotrypsin inhibitor and a pharmaceutically acceptable carrier.

In another aspect, the invention features a pharmaceutical composition comprising consisting essentially of or consisting of a chymotrypsin inhibitor and a pharmaceutically acceptable carrier. The composition can include a polymer that controls the release of the inhibitor. The composition can include a second agent, e.g., a GC-C receptor agonist such as guanylin or a biologically active variant or fragment thereof.

In another aspect, the invention features a method for treating obesity, the method comprising administering a pharmaceutical composition comprising consisting essentially of or consisting of a chymotrypsin inhibitor and a pharmaceutically acceptable carrier. The composition can be administered in combination with another agent for treatment of obesity.

In another aspect, the invention features a method for treating congestive heart failure, the method comprising: administering to the patient a pharmaceutical composition comprising consisting essentially of or consisting of a chymotrypsin inhibitor and a pharmaceutically acceptable carrier. The composition can be administered in combination with another agent for treatment of congestive heart failure, for example, a natriuretic peptide such as atrial natriuretic peptide, brain natriuretic peptide or C-type natriuretic peptide, a diuretic, or an inhibitor of angiotensin converting enzyme.

In another aspect, the invention features a method for treating benign prostatic hyperplasia, the method comprising: administering to the patient a pharmaceutical composition comprising consisting essentially of or consisting of a chymotrypsin inhibitor and a pharmaceutically acceptable carrier. The composition can be administered in combination with another agent for treatment of BPH, for example, a 5-alpha reductase inhibitor (e.g., finasteride) or an alpha adrenergic inhibitor (e.g., doxazosine).

The chymotrypsin inhibitor can be administered in combination with guanylin or a biologically active fragment or variant thereof. A number of guanylin and variants thereof are depicted in FIGS. 3-5. Also useful are guanylin related polypeptides having the formula: In a first aspect, the invention features a polypeptide comprising, consisting of, or consisting essentially of the amino acid sequence: Xaa₁ Xaa₂ Xaa₃ Cys₄ Xaa₅ Xaa₆ Xaa₇ Xaa₈ Xaa₉ Xaa₁₀ Xaa₁₁ Cys₁₂ Xaa₁₃ Xaa₁₄ Xaa₁₅ Xaa₁₆ (SEQ ID NO: 1) wherein:
Xaa₁ is Ser, Asn, Tyr, Ala, Gln, Pro, Lys, Gly, or Thr, or is missing;
Xaa₂ is His, Asp, Glu, Ala, Ser, Asn, Gly, or is missing;
Xaa₃ is Thr, Asp, Ser, Glu, Pro, Val or Leu;
Xaa₅ is Asp, Ile or Glu;
Xaa₆ is Ile, Trp or Leu;
Xaa₇ is Cys, Ser, or Tyr;
Xaa₈ is Ala, Val, Thr, Ile, Met or is missing;
Xaa₉ is a) any amino acid, b) Phe, Tyr, Asn, Trp, c) an amino acid other than Phe, Trp, or Tyr, d) non-aromatic amino acid or e) is missing;
Xaa₁₀ is Ala, Val, Met, Thr or Ile;
Xaa₁₁ is Ala or Val;
Xaa₁₃ is Ala or Thr;
Xaa₁₄ is Gly, Ala or Ser;
Xaa₁₅ is Cys, Tyr or is missing; and
Xaa₁₆ is: a) any amino acid; c) is missing or d) His or Leu or Ser.

The details of one or more embodiments of the invention are set forth in the accompanying description and claims. The publications and patents referenced herein are incorporated by reference.

### FIGURES

FIG. 1 shows the results of a T84 cGMP assay to assess guanylin activity after a chymotrypsin digestion assay. The chymotrypsin digestion assay was performed in the presence or absence of chymostatin.

FIG. 2 shows the results of LC/MS analysis of the processing of guanylin in a chymotrypsin digestion assay. The chymotrypsin digestion assay was performed in the presence or absence of chymostatin.

FIG. 3 depicts the amino acid sequence of various deletion variants of human guanylin in which one, two, three or four amino acids are deleted. The deleted amino acids are between the first and fourth cysteines in human guanylin as well as amino terminal to the first cysteine in human guanylin.

FIG. 4 depicts the amino acid sequence of various insertion variants of human guanylin in which one, two, three or four amino acids are inserted. The inserted amino acids are between the first and fourth cysteines in human guanylin as well as amino terminal to the first cysteine in human guanylin and carboxy terminal to the fourth cysteine in human guanylin.

FIG. 5 depict the amino acid sequence of various guanylins and variants thereof.

### DETAILED DESCRIPTION

Guanylin binds to and activates the guanylate cyclase (GC-C) receptor, a key regulator of fluid and electrolyte balance in the intestine and kidney. When stimulated, this receptor, which is located on the apical membrane of the intestinal epithelial surface, causes an increase in intestinal epithelial cyclic GMP (cGMP). This increase in cGMP is believed to cause a decrease in water and sodium absorption and an increase in chloride and potassium ion secretion, leading to changes in intestinal fluid and electrolyte transport and increased intestinal motility. The intestinal GC-C receptor possesses an extracellular ligand binding region, a transmembrane region, an intracellular protein kinase-like region and a cyclase catalytic domain. Proposed functions for the GC-C receptor are fluid and electrolyte homeostasis, the regulation of epithelial cell proliferation and the induction of apoptosis (Shaibhubhai 2002 Curr Opin Drug Dis Devel 5:261-268).

In the human body an inactive form of chymotrypsin, chymotrypsinogen is produced in the pancreas. When this inactive enzyme reaches the small intestine it is converted to active chymotrypsin by the excision of two di-peptides. Active chymotrypsin will cleave peptides at the peptide bond on the carboxy-terminal side of Trp, Tyr or Phe and can cleave a peptide at the peptide bond on the amino terminal side of a Leu, Ile or Val (and, at elevated pH, His). Thus, chymotrypsin can cleave guanylin as shown by Greenberg et al. (J Investig Med 45:276-82, 1997). Chymotrypsin-mediated inactivation of guanylin can be prevented by chymostatin, a chymotrypsin inhibitor. Santos-Neto et al 2003 Pharm & Toxicol 92:114 observed that chymostatin was required in order to observe guanylin-induced intestinal fluid secretion in the suckling mouse model.

### Chymotrypsin Inhibitors

A wide variety of peptide and non-peptide chymotrypsin inhibitors are known. For example:
1. tissue-factor-pathway inhibitor (TFPI) (Peterson et al 1996 Eur J Biochem 235:310-6; for examples see human (GENBANK® AAH15514 GI:15930156), mouse (GENBANK® AAH36146 GI:23271605), and dog (GENBANK® AAB32443 GI:833924));
2. α-2 antiplasmin (Potempa et al. 1988 Science 241: 699-700, GENBANK® Accession P08697, GI:112907, SEQ ID NO. XXXMALLWGLLVLSWSCLQGPCSVFSPVSAMEPLGRQLTSGPNQEQVSPLTLLKLGN QEPGGQTALKSPPGVCSRDPTPEQTHRLARAMMAFTADLFSLVAQTSTCPNLILS PLSVALALSHLALGAQNHTLQRLQQVLHAGSGPCLPHLLSRLCQDLGPGAFRLA ARMYLQKGFPIKEDFLEQSEQLFGAKPVSLTGKQEDDLANINQWVKEATEGKIQ EFLSGLPEDTVLLLLNAIHFQGFWRNKFDPSLTQRDSFHLDEQFTVPVEMMQART YPLRWFLLEQPEIQVAHFPFKNNMSFVVLVPTHFEWNVSQVLANLSWDTLHPPL VWERPTKVRLPKLYLKHQMDLVATLSQLGLQELFQAPDLRGISEQSLVVSGVQH QSTLELSEVGVEAAAATSIAMSRMSLSSFSVNRPFLFFIFEDTTGLPLFVGSVRNP NPSAPRELKEQQDSPGNKDFLQSLKGFPRGDKLFGPDLKLVPPMEEDYPQFGSPK );
3. members of the serpin α-1 antichymotrypsin family (Forsyth et al. 2003 Genomics 81: 336-45; for example see CAS Registry No. 141176-92-3; functional variants thereof are described in European patent application EP1415664 and in Plotnick et al. 2003 Biochemistry 33:29927 (for example the P2 (Leu-357) variant);
4. gelin (U.S. Pat. No. 5,397,694, partial sequence (aa 1-29) can be found at GENBANK® Accession AAB27871, GI:409493, SEQ ID NO. XXXVDEKAEVTDGLCGDWTCSGAQVXQNDAAV), which has been proposed as a treatment for dermatitis as well a periodontitis and gingivitis;
5. hirustasin (Sollner et al. 1994 Eur J Biochem. 219: 937-43, GENBANK® Accession No P80302, GI:461516, SEQ ID NO. XXXTQGNTCGGETCSAAQVCLKGKCVCNEVHCRIRCKYGLKKD ENGCEYPCSCAKASQ);
6. certain eglins including eglin C (GENBANK® Accession P01051, GI:124128, SEQ ID NO. XXXTEFGSELKSFPEVVGKTVDQAREYFTLHYPQYDVYFLPEGSPVTLDLRYNRVRVF YNPGTNVVNHVPHVG) are peptide inhibitors of chymotrypsin. For other examples of eglins, see those disclosed in U.S. Pat. No. 5,180,667, U.S. Pat. No. 6,342,373, U.S. Pat. No. 4,636,489, Seemuller et al. 1981 Methods Enzymol. 804-816, Seemueller et al. 1986 Research Monographs in Cell and Tissue Physiology 337-59, Nick et al 1988 Adv in Experimental Medicine and Biology 240:83-8, and Schnebli et al 1986 Pulm. Emphysema Proteolysis (conference) CAN 107:228147 AN 1987:628147), which has been considered as a treatment for emphysema and for use as a non-steroidal anti-inflammatory agent;
7. inhibitors from Bombyx mori (see, e.g., JP 4013698 A2 and JP 04013697 A2; CA registry No. 142628-93-1, SEQ ID NO. XXX DEPTTKPFCEQAFGDCGTPY and CA registry No. 142628-94-2, SEQ ID NO. XXX DKPTTEPFIC EQRFGNCGTG);
8. the leech derived peptide thrombin inhibitor, hirudin (Zwilling 1968 Hoppe-Seyler's Zeitschriftfuer Physiologische Chemie 349:1787-8, CA Registry No. 8001-27-2, see for example, Genbank AAA01384 GI:269388, SEQ ID NO. XXXITYTDCTESGQNLCLCEGSNVCGKGNKCILGSQGKDNQCVTGEGTPKPQSHNQG DFEPIPEDAYDE). Hirudin variants are disclosed in the literature (for examples see those in U.S. Pat. No. 5,674,838, Great Britain patent application GB2242681 and those described in Wirsching et al 2003 Molecular Genetics and Metabolism 80:451-462); 9. a shorter hirudin variant, hirulog/BG 8967 (CA Registry No. 128270-60-0, SEQ ID NO. XXX; FPRPGGGGNGDFEEIPEEYL; Angiomax® (bivalirudin)) may also have chymotrypsin inhibition activity and may thus be useful in the present invention along with other peptides disclosed in PCT publication WO04076484 and U.S. Pat. No. 5,196,404; and
10. secretory leukocyteprotease inhibitor (SLPI) (for examples see GENBANK® CAA28187 GI:758101 (human), GENBANK® NP_{—}445824.1 GI:16758102 (rat), and GENBANK® NP_{—}035544.1 GI:6755574 (mouse); also Farley et al 1997 Drugs and the Pharmaceutical Sciences 84:305-334.
11. α-1 anti-trypsin which can inhibit elastase as well as chymotrypsin and thus may be useful in the present invention (for examples see GENBANK® CAB06092 GI:2780174 (human) and GENBANK® NP_{—}001009663 GI:57527135 (rat)). This product has been sold to treate α-1 anti-trypsin deficiency (a genetic disorder) as . Zemaira™ (Aventis Behring; FDA biologics license 2003 License #1281), Prolastin® (Bayer), and Aralast™ (Baxter).

In addition, a large number of peptide inhibitors of chymotrypsin are reviewed by Schoofs et al. (2002 Curr Pharm Des. 8: 483-91) and by Salier et al. (1996 Biochem J 315:1-9). McBride et al. 1996 J Mol Biol. 259: 819-27 and McBride et al. 2000 J Pept Sci. 6:446-52 disclose chymotrypsin inhibitors derived from combinatorial peptide libraries including those represented by CAS registry Nos. 306762-66-3, 306762-67-4, 306762-68-5, 306762-69-6, 306762-70-9, 306762-71-0, 306762-72-1, 306762-73-2, 306762-74-3, 306762-75-4, 178330-92-2, 178330-93-3, 178330-94-4, and 178330-95-5.

In addition, various small molecule inhibitors of chymotrypsin have been described including:
1. the compound depicted below and described in EP 0071433 (CAS registry No. 81459-62-3)

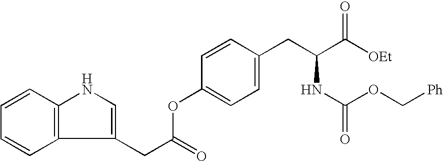

Other chymotrypsin inhibitors disclosed in EP 0071433 include those specifically identified as chymotrypsin inhibitors on pages 11-16 of the application and those identified by CA registry Nos. 81459-79-2, 81460-01-7, 85476-59-1, 85476-62-6 (also known as FK-401 CA Index name IH-Indole-3-acetic acid, 5-methoxy-2-methyl-, 4-[[2-[4-[2-(4-morpholinyl)ethyl]-1-piperazinyl]ethoxy]carbonyl]phenyl ester, trihydrochloride), 85476-63-7, 85476-67-1, 85476-70-6, 85858-66-8, 85858-68-0, 85858-69-1, 85858-70-4, 85858-71-5, 85858-72-6, 85858-73-7, 85858-75-9, 85858-77-1, 85858-79-3, 85858-81-7, 85858-83-9, 85858-84-0, 85858-85-1, 85858-87-3, 85858-89-5, 85858-90-8, 85858-92-0, 85879-03-4, 85879-05-6, 85879-06-7, and 85879-08-9);
2. compounds with chymotrypsin inhibition activity described in JP 56092217 A2;
3. compounds with chymotrypsin inhibition activity described in U.S. Pat. No. 4,755,383 (including, 1-Naphthaleneacetic acid, 4-[[4-(1-methylethyl)-1-piperazinyl]carbonyl]phenyl ester (CA registry No. 90186-24-6), Acetic acid, 9H-fluoren-9-ylidene-, 4-[[4-(1-methylethyl)-1-piperazinyl]carbonyl]phenyl ester (CA registry No. 90185-93-6), 1-Naphthalenecarboxylic acid, 1,2,3,4-tetrahydro-4-[[4-(1-methylethyl)-1-piperazinyl]carbonyl]phenyl ester, monomethanesulfonate (CA registry No. 89703-10-6), 1-Naphthalenecarboxylic acid, 1,2,3,4-tetrahydro-, 4-[(4-cyclohexyl-1-piperazinyl)carbonyl]phenyl ester (CA registry No. 85858-74-8), and 1-isopropyl-4-[4-(1,2,3,4-tetrahydronaphthoyloxy) benzoyl]piperazine methanesulfonate).
4. chymotrypsin inhibitors described in U.S. Pat. Nos. 4,755,383 and 4,639,435 include those of Formula I:

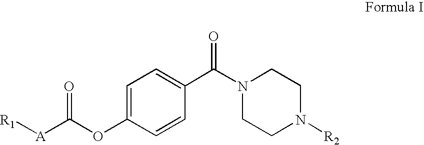

wherein A is a single bond, or an alkylene, vinylene, -0-alkylene or methine group; R¹ is a bicyclic carbocyclic residue which may partly be saturated and may optionally be substituted by at least one member of the class consisting of lower alkyl, lower alkoxy, oxo and nitro groups and halogen atoms; a fluorene residue which may optionally have an oxo group; a fluorenylidene group; an anthracene residue; a phenanthrene residue which may partly be saturated and may optionally be substituted by at least one lower alkyl group; a benzofuran or thianaphthene residue which may optionally be substituted by at least one member of the class consisting of lower alkyl and lower alkoxy groups; a benzopyran or benzazine residue which may partly be saturated and may optionally be substituted by at least one member of the class consisting of oxo and phenyl groups; a phthalimide residue; a benzodiazone residue; an isoxazole residue which may optionally be substituted by at least one member of the class consisting of lower alkyl and phenyl groups; an alkylenedioxybenzene residue; or a xanthene residue, and R² is a loweralkyl, cycloalkyl, cycloalkylalkyl or aralkyl group.

The methods of producing the compounds of the formula (I) and their chymotrypsin inhibitory activity characteristics have been described in Japanese Patent Application No. 109192/1982 and in Japanese Patent Application filed Apr. 28, 1983.
5. inhibitors described in EP 0128007 and U.S. Pat. No. 4,620,005 including: 1-isopropyl-4-[(4-(1,2,3,4-tetrahydro-1-naphthoyloxy)phenyl)carbonyloxymethyl carbonyl]piperazine; 1-isopropyl-4-[(4-(1,2,3,4-tetrahydro-1-naphthoyloxy)phenyl)ethyl carbonyl]piperazine; and and 1-isopropyl-4-[(4-(1,2,3,4-tetrahydro-1-naphthoyloxy)phenyl)methylcarbonyl ]piperazine;
6. inhibitors described U.S. Pat. No. 4,898,876 including inhibitors: 1-isopropyl-4-(4-(5,6,7,8-tetraphydronaphthalene-1-acetyloxy)benzoyl)piperazinehydrochloride; 1-isopropyl-4-(4-(9-fluorenylidene acetyloxy)benzoyl)piperazine; 1-isopropyl-4-(4-(thianaphthene-2-acetyloxy)benzoyl)piperazine methanesulfonate; 1-methyl-4-(4-(7-methoxyl-1,2,3,4-tetrahydro-1-naphtoyloxy) benxoyl)piperazine;methanesulfonate; and 1-methyl-4-(3-(1,2,3,4-tetrahydro-1-naphthoyloxy)benzoyl)piperazine hydrochloride);
7. YS3025 (CAS Registry No. 138320-33-9) disclosed by Rizzi et al. (1992 Biochem Int. 28:385-92);

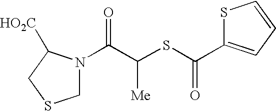

8. MR889 (CA registry No. 94149-41-4) disclosed by Luisetti et al. (1989 Biochem Biophys Res Commun. 165:568-73);

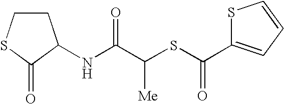

9. chymotrypsin inhibitors disclosed by Yokoo et al. (1987 Yakugaku Zasshi 107:732-7), many of which are of the phenyl ester type and which include those represented by CAS Registry Nos. 85858-76-0, 89703-10-6 (also known as FK-448 CA index name 1-Naphthalenecarboxylic acid, 1,2,3,4-tetrahydro-, 4-[[4-(1-methylethyl)-1-piperazinyl]carbonyl]phenyl ester, monomethanesulfonate), 90185-92-5, 90185-96-9, 90185-98-1, 90186-00-8, 90186-01-9, 90186-05-3, 90186-06-4, 90186-07-5, 90186-08-6, 90186-09-7, 90186-10-0, 90186-11-1, 90186-12-2, 90186-13-3, 90186-14-4, 90186-22-4, 90186-23-5, 90186-24-6, 90186-25-7, 90186-27-9, 90186-28-0, 90186-29-1, 90186-31-5, 90186-35-9, 90186-43-9, 90209-88-4, 90209-89-5, 90209-92-0, 90209-94-2, 90209-96-4, 90209-97-5, 90210-01-8, 90210-03-0, 90210-04-1, 90210-25-6, 90210-26-7, 90210-28-9, 90230-84-5, 90409-84-0, 95460-86-9, 95460-87-0, 95460-88-1, 95460-89-2, 95460-91-6, 114949-00-7, 114949-01-8, 114949-02-9, 114949-03-0, 114949-04-1, 114949-05-2, 114949-06-3, 114949-18-7, 114949-19-8, 114964-69-1, and 114964-70-4; and
10. the two chymotrypsin inhibitors shown below described by Boulanger (1986 Journal of Medicinal Chemistry 29:1483-7);

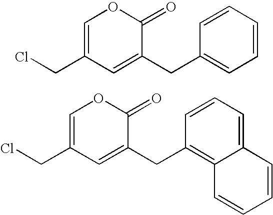

A number of chymotrypsin inhibitors are available from commercial suppliers, including:
1. chymostatin (CAS registry No. 9076-44-2);

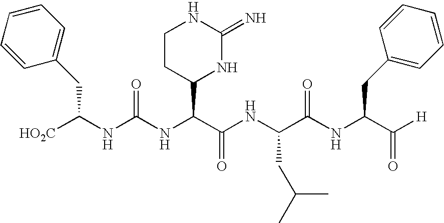

2. the inhibitor aprotinin or a derivative thereof (TRASYLOL®); CAS Registry No. 9087-70-1; SEQ ID NO. XXX; MKMSRLCLSVALLVLLGTLAASTPGCDTSNQAKAQRPDFCLEPPYTGPCKARIIR YFYNAKAGLCQTFVYGGCRAKRNNFKSAEDCMRTCGGAIGPWENL);
3. 4-(2-aminoethyl)-benzenesulfonylfluoride hydrochloride (Pefabloc; CAS Registry No. 30827-99-7);

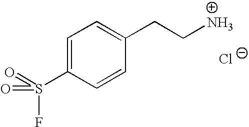

4. benzamidine (CA Registry No. 618-39-3);

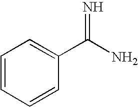

5. di-isopropyl phosphofluoridate (CAS Registry No. 55-91-4);

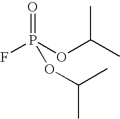

6. 6-aminocaproic acid (CAS Registry No. 60-32-2);

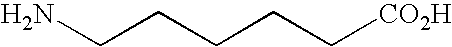

7. CAS Registry No. 88070-98-8;

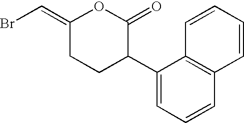

8. ecotin (a peptide inhibitor, CAS Registry No. 87928-05-0; ecotin variants with enhanced activity are described in PCT Publication Nos. WO0061634 and WO0061782);
9. PMSF;

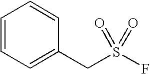

and
10. benzenesulfonamide (also known as N-tosyl-L-phenylalaninechloromethyl ketone (TPCK); CAS Registry No. 402-71-1).

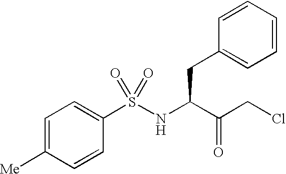

A number of chymotrypsin inhibitors are produced by plants, including: the peptide inhibitor CI2 (CA Registry No. 139466-47-0, GENBANK® Accession S18818, GI:100574), and variants and homologs thereof including CI-2A (U.S. Pat. No. 5,167,483), CI-2A (WO 9205239), WCI-3 (Shibata et al. 1988 J Biochem (Tokyo) 104:537-43), WCI-2 (Habu et al. 1992 J Biochem (Tokyo) 111:249-58), and WCI-x (Habu et al., supra). Other plant-derived inhibitors have also been described (Bryant et al. 1976 Biochemistry 15:3418-24; Hass et al. 1982 Biochemistry 21:752-6; Birk 1985 Int J Pept Protein Res. 25:113-31; Pearce et al 1982 Archives of Biochemistry and Biophysics 213:456-62; Tamir et al 1996 Journal ofProtein Chemistry 15: 219-29; Birk et al 1999 Khimiya beYisra'el 1:9-12; Polya 2003 Studies in Natural Products Chemistry 29: 567-641; Weder et al 2004 Journal of Agricultural and Food Chemistry 52:4219-4226; Teles et al 2004 Phytochemistry 65: 793-799; Tsoi et al 2004 Biological Chemistry 385:185-189 and Kollipara et al. 1992 Journal of Agricultural and Food Chemistry 40:2356-63). Hammond et al 1984 J. Biol. Chem. 259: 9883-9890 describe the Bowman-Birk protease inhibitor (BBI) in soybean (for example see GENBANK® AA089509 GI:29691202). BBIs are reviewed in Bowman et al. 1993 Protease Inhib. Cancer Chemoprev. Agents 93-6. Synthetic peptides have been generated which mimic BBI inhibitors (McBride et al 2001 Current Medicinal Chemistry 8: 909-917 and McBride et al 2002 Biopolymers 66:79-92). Other chymotrypsin inhibitors are described in Bister et al 2004 Journal of Natural Products 67: 1755-1757; Szenthe et al 2004 Biochemistry 43:3376-3384; Zhou et al. 2004 Comparative Biochemistry and Physiology, Part B: Biochemistry & Molecular Biology 137B:219-224 and Mak et al. 2004 Biochimica et Biophysica Acta 1671:93-105. These chymotrypsin inhibitors and others are useful in the methods of the invention.

### Chymotrypsin Activity Assays

Any standard chymotrypsin activity assay can be used to assess known chymotrypsin inhibitors and compounds which may inhibit chymotrypsin.

For example chymotrypsin activity can be measured using N-Glutaryl-Lphenylalanine p-nitroanilide (Sigma-Aldrich, Inc; Catalog No. 49738) as a substrate and cc-Chymotrypsin from bovine pancreas (EC 3 2 1. 1; Sigma-Aldrich; Catalog No. C4129) in an assay described by Kakade et al. (Cereal Chemistry 51:376 (1974)). In this assay, chymotrypsin hydrolyzes the substrate N-Glutaryl-L-phenylalanine-p-nitroanilide present in excess. The release of p-nitroanilide, a yellow dye, is measured spectrophotometrically.

An additional chymotrypsin assay is described by Kourteva et al. (Analytical Biochemistry 162:345-9, 1987) This assay is rapid and particularly useful for assessing higher molecular weight inhibitors. Briefly, a test compound is spotted onto an agar film which contains TLCK-chymotrypsin. Enzyme inhibition is visualized as colorless zones on a pink background after the films are stained with a chromogenic substrate N-acetyl-DL-phenylalanine-β-naphthyl ester. A variation on this assay can be use to assess trypsin activity and this can be useful for assessing the selectivity of an inhibitor.

### Guanylin Degradation Assay

Since guanylin is susceptible to digestion by chymotrypsin, chymotrypsin activity can be assayed based on guanylin cleavage. Guanylin (Sigma, G-116) or a guanylin variant were resuspended in 5 ml of 100 mM Tris-HCl, 2 mM CaCl₂, pH 7.8 at 30° C. for a final concentration of 0.01 mg/ml. From this stock, six (6) 500 μl aliquots were prepared in 2 ml Eppendorf tubes and labeled “Control”, “T0”, “T15”, “T30”, “T60”, and “T180”, with “T_” representing timepoints, in minutes. 5 μl of a 10 mM chymostatin (Sigma, C7268) stock (5 mg of chymostatin resuspended in 824 μl of DMSO) was added to the “Control” samples and all samples were incubated for 5 minutes at 30° C. Chymostatin inhibits chymotrypsin activity and this sample served as a negative control. Next, 20 μl of a chymotrypsin (Sigma, C6423) enzyme solution (0.01 mg/ml bovine chymotrypsin enzyme in 1 mM HCl, 2 mM Calcium Chloride) were added to the samples and mixed by inversion. Samples are incubated at 30° C. The “T0” samples were collected at time=0 minutes by adding 5 μl of a 10 mM chymostatin stock and subsequently stored at −80° C. All other timepoint samples are taken in a similar manner, with the “Control” samples collected parallel to the time=180 minutes samples. Determination of sensitivity to digestion by chymotrypsin was determined by LCMS analysis and by in vitro activity in the T84 cGMP assays described below.

For LC/MS analysis, samples were injected (10 μL) onto a reverse phase HPLC column (Waters Atlantis dC₁₈1.0×150 mm, 3 um particle size, 186001283) at 20° C., and were eluted with a reverse phase gradient (Mobile Phase A: 5 mM NH₄OAc in dH₂O, 0.1% formic acid, Mobile Phase B: 5 mM NH₄OAc in 80% methanol and 20% dH₂O, 0.1% formic acid; Initial condition of 5% B, ramping to 95% B over 35 minutes, and holding for 3 minutes, then returning to initial conditions over the next 7 minutes, all at a flow rate of 0.07 mL/min.). At 45 minutes, the gradient was at initial conditions of 5%B and held for 15 minutes. Guanylin samples were detected by quadrapole-time of flight mass spectrometry in TOF scan mode (cone voltage=30 V; collision=4 eV). Chymotrypsin sensitivity was determined by the loss of the initial mass species and the formation of the product mass species, with respect to time. Instrument response was converted into percentage units by comparison of the response of the initial mass versus the product mass, with “T0” representing total response of initial mass for all samples.

### Guanylins and Guanylin Variants

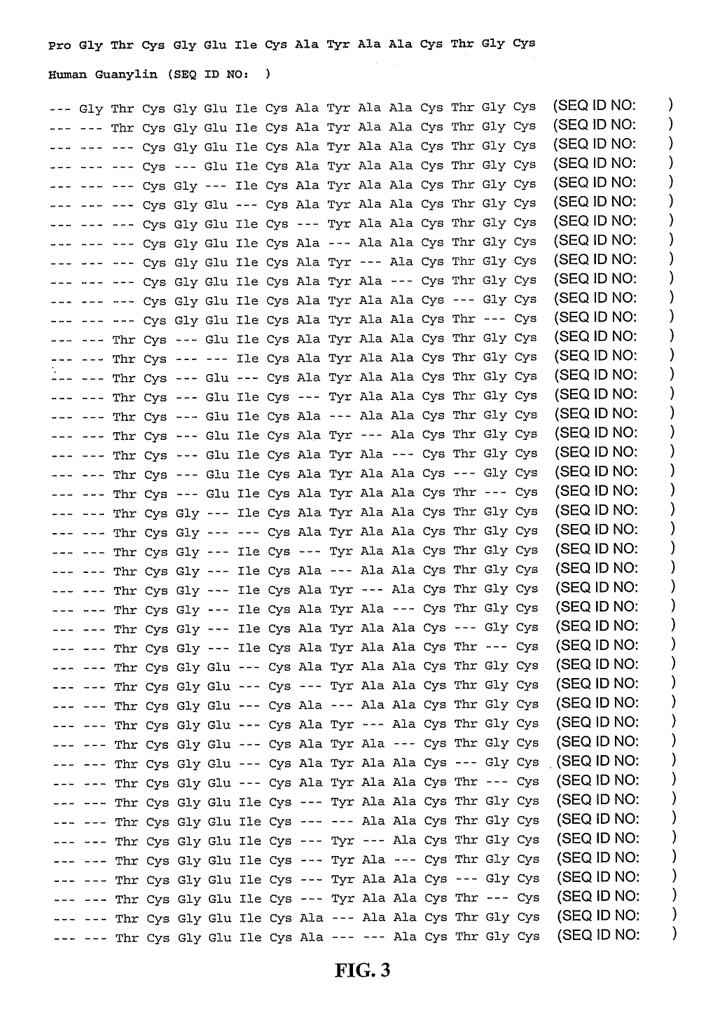

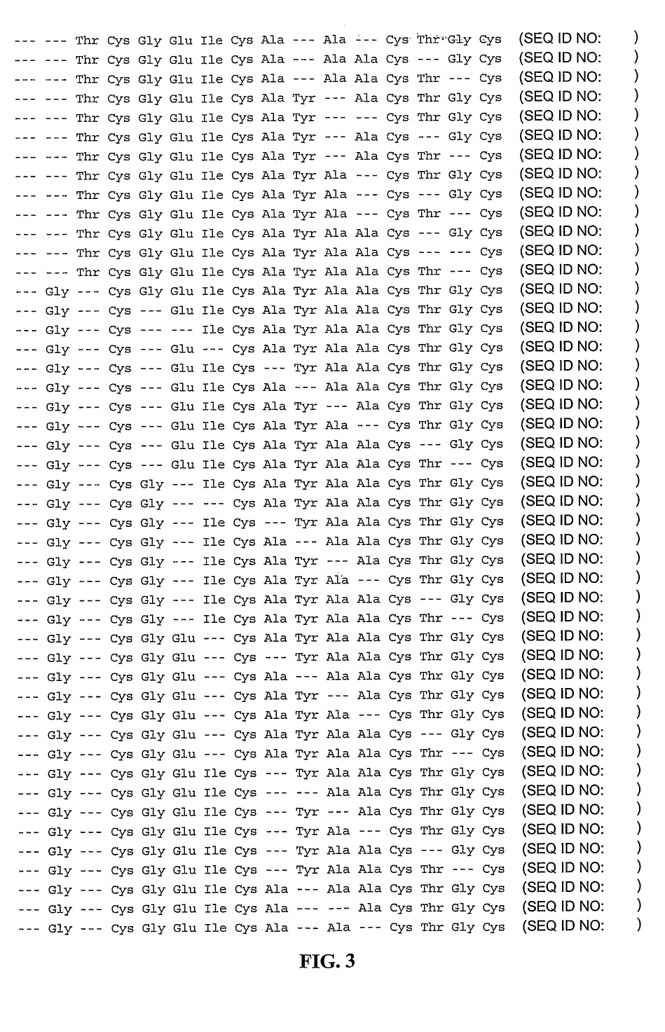

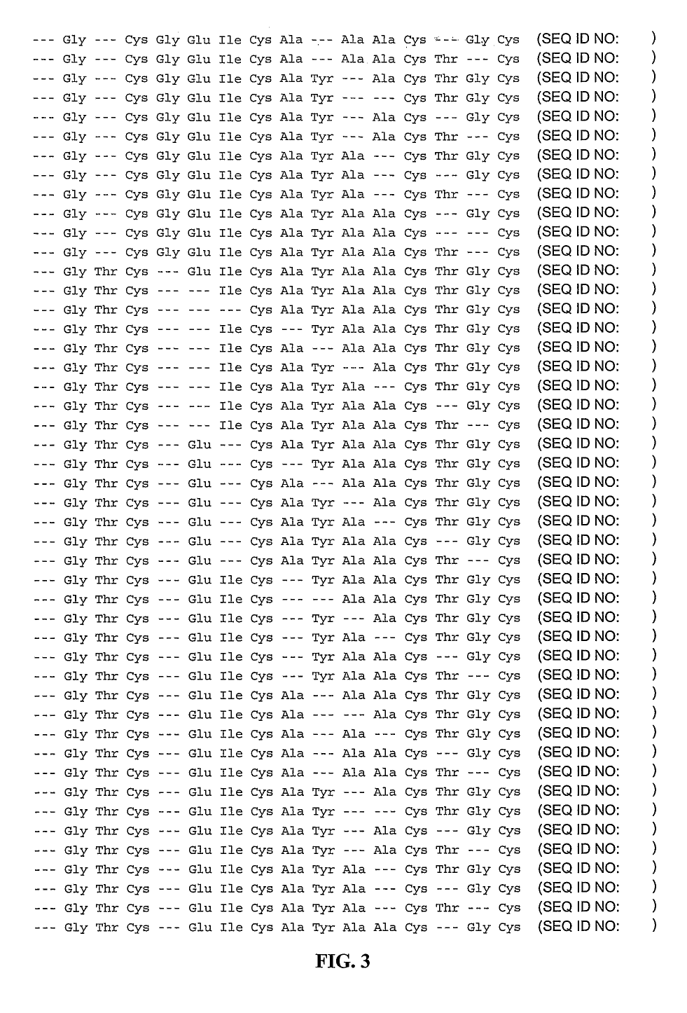

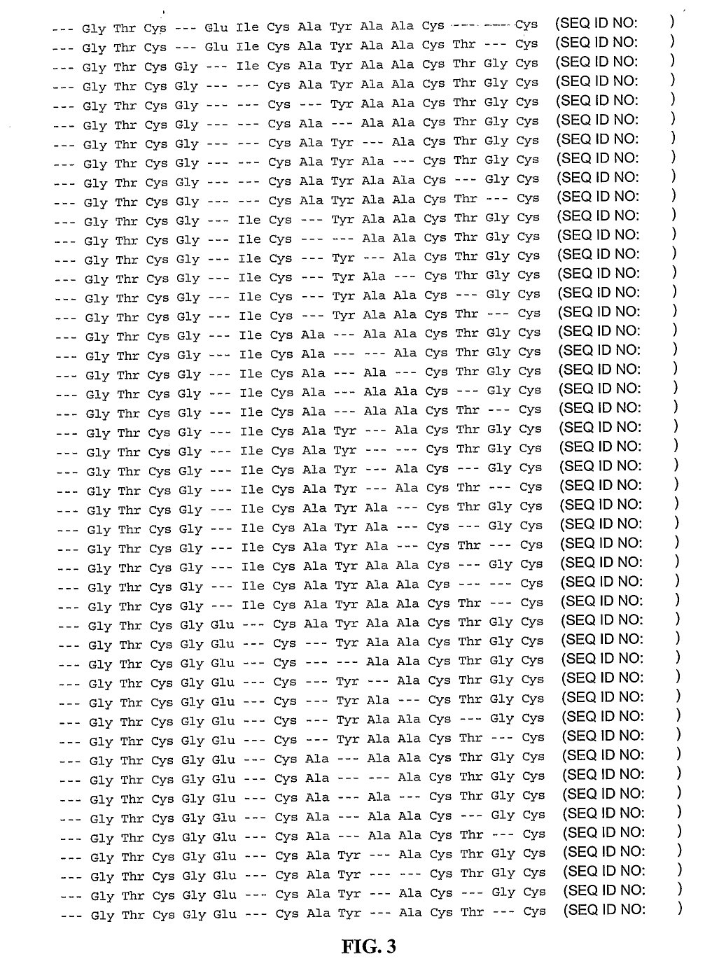

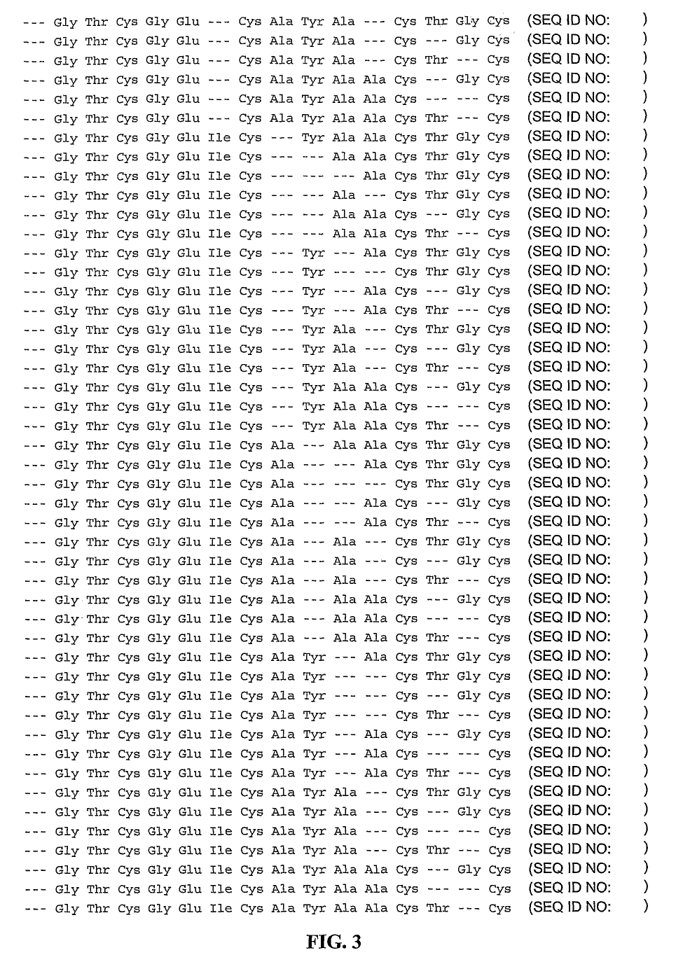

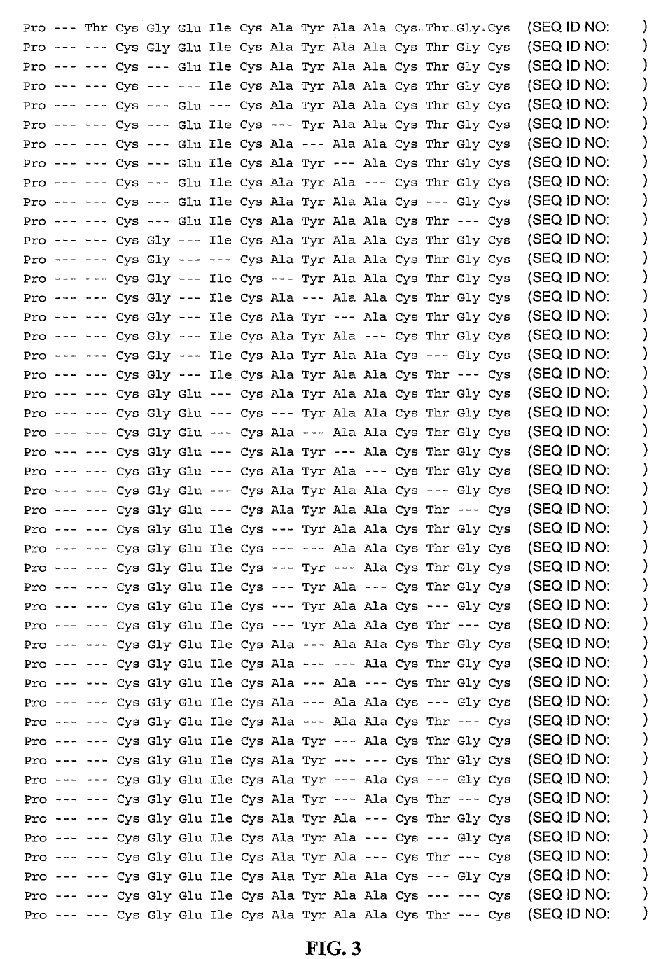

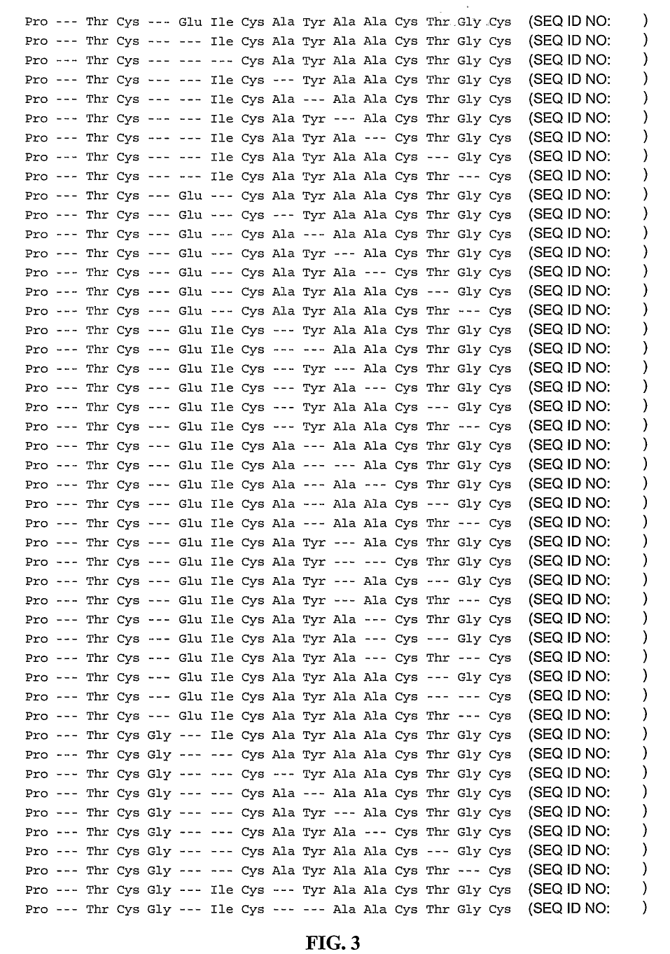

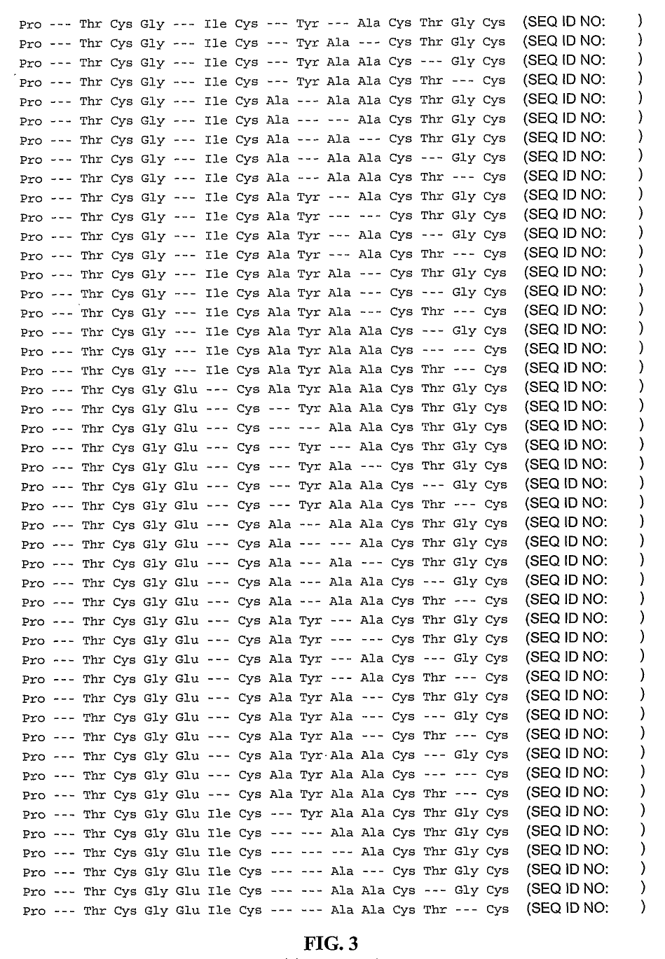

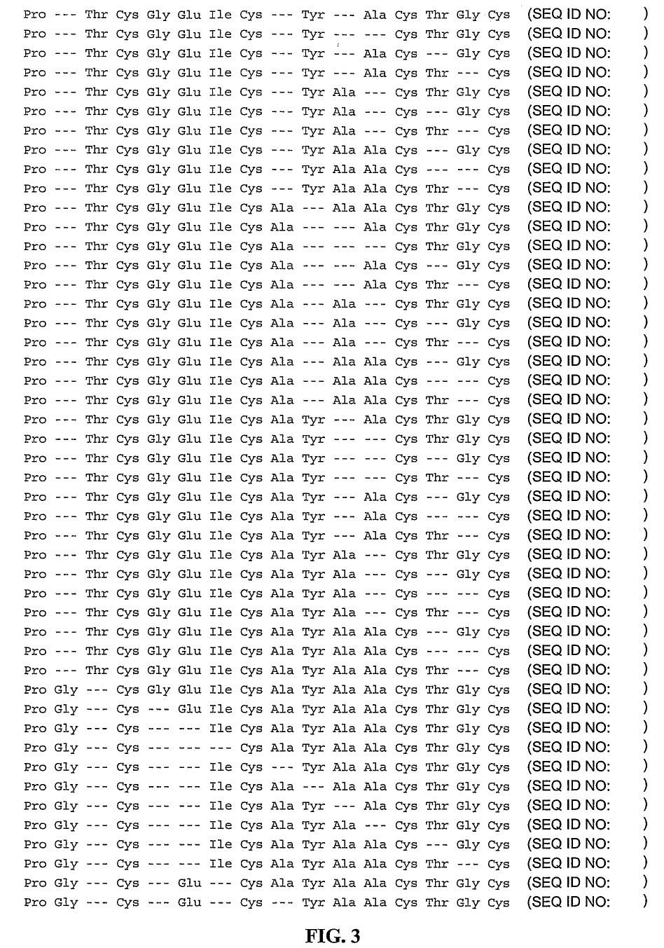

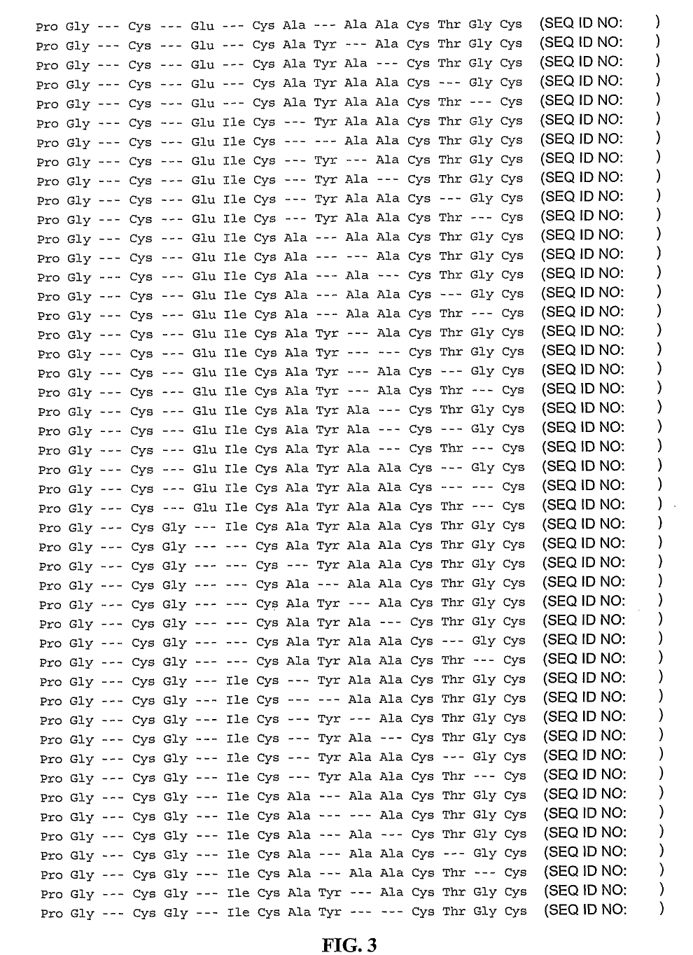

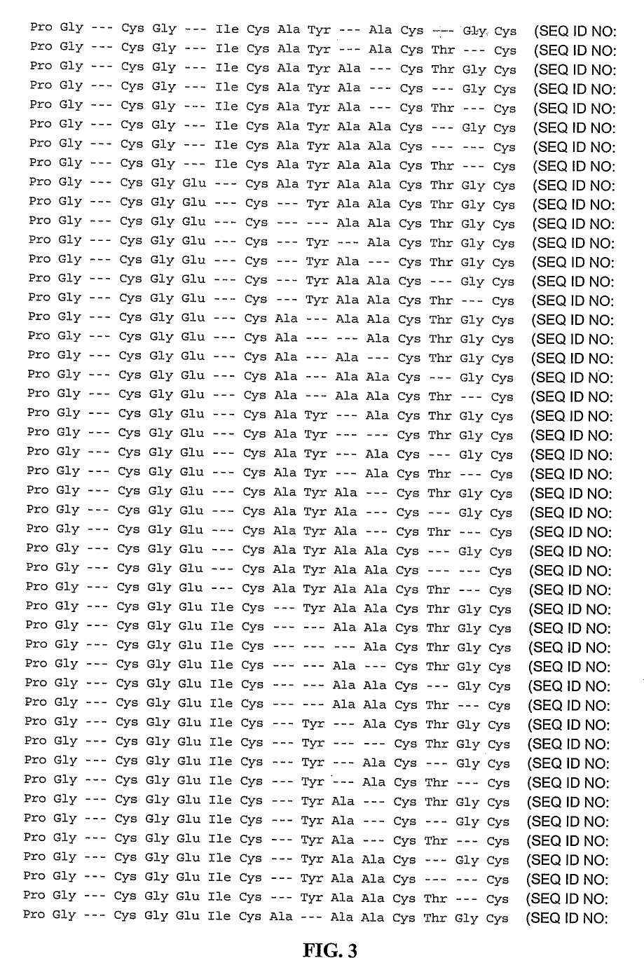

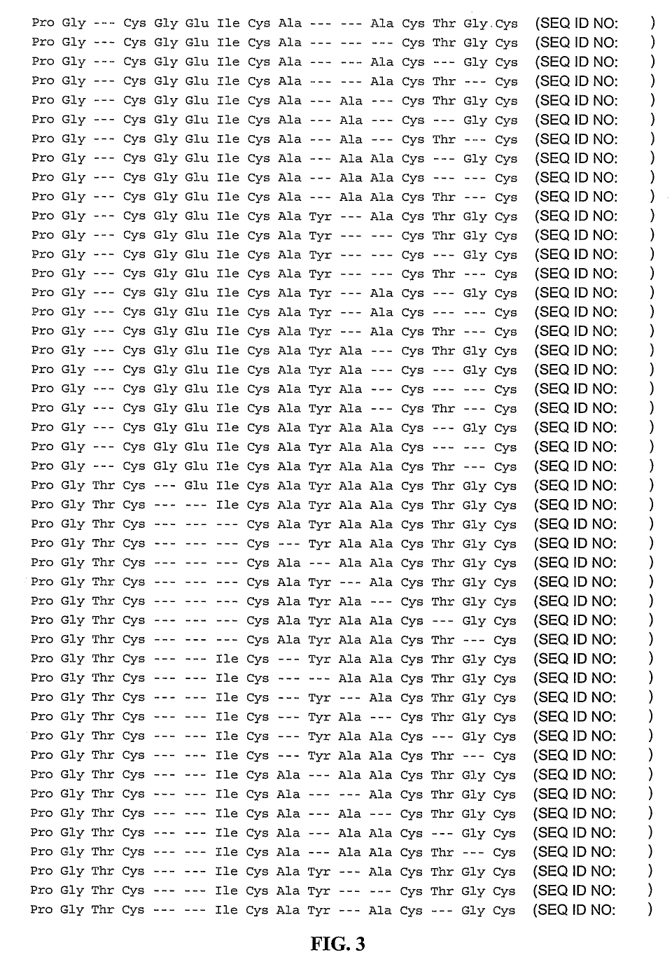

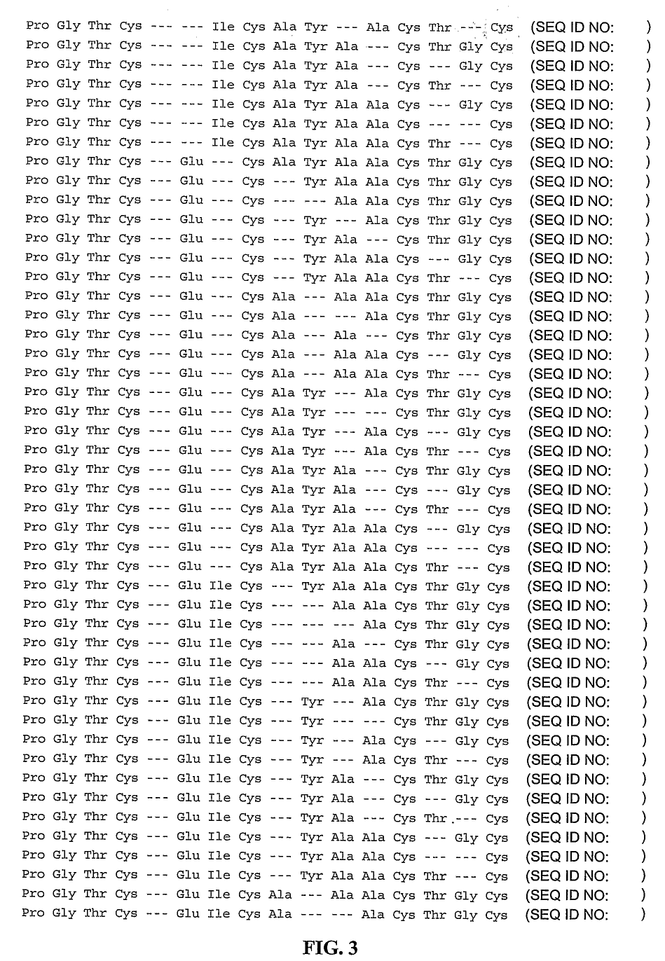

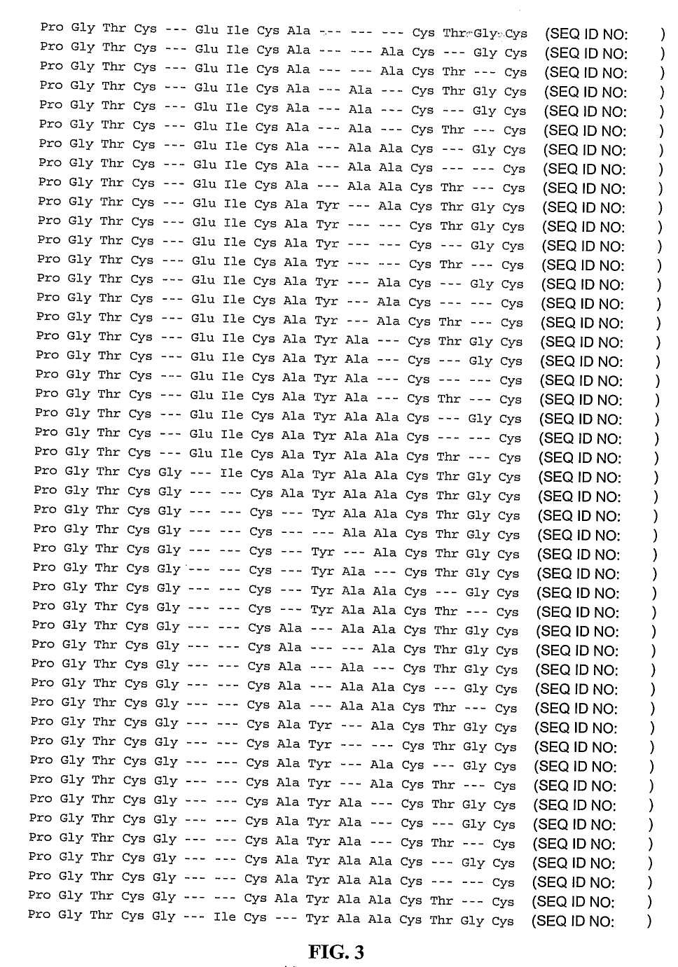

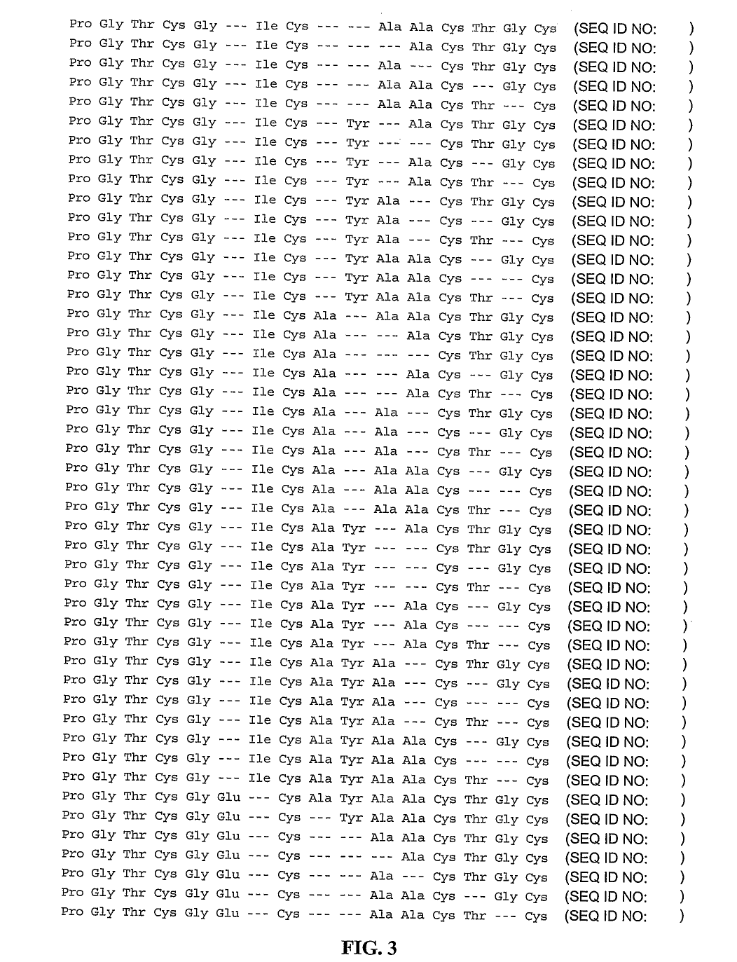

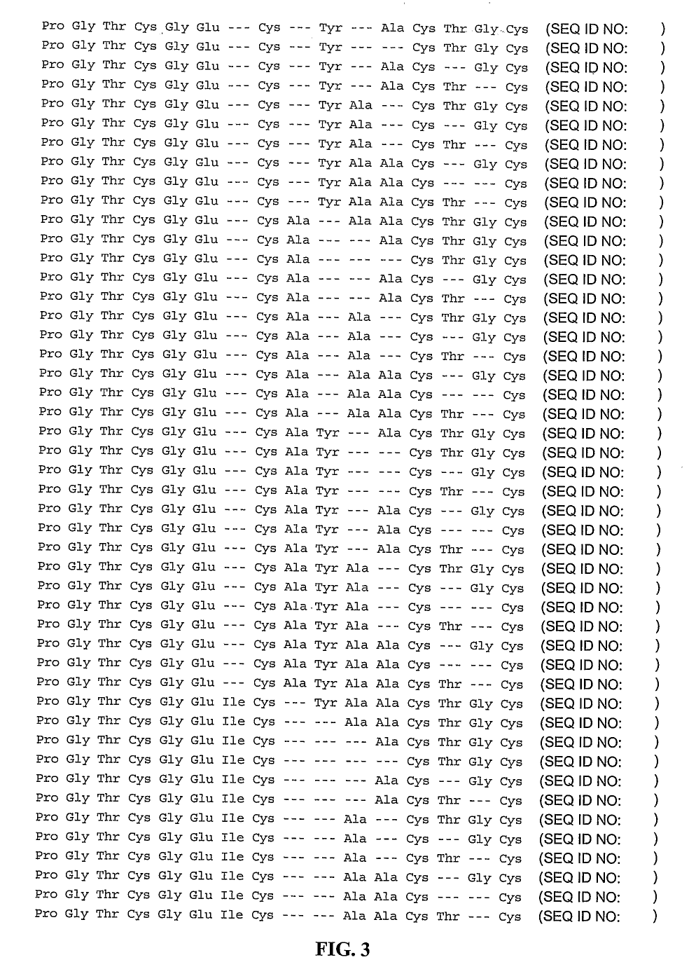

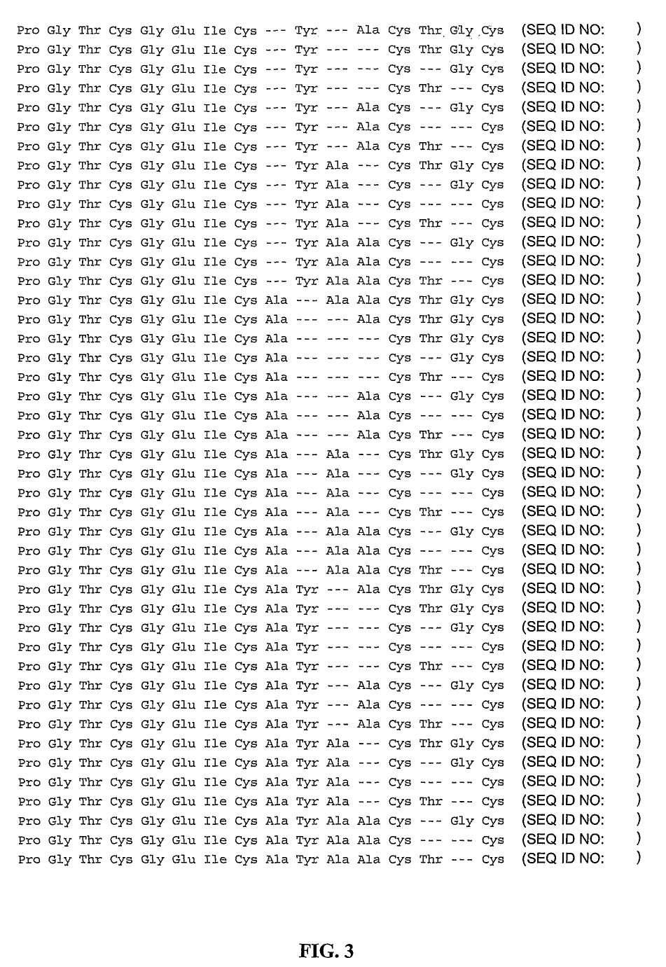

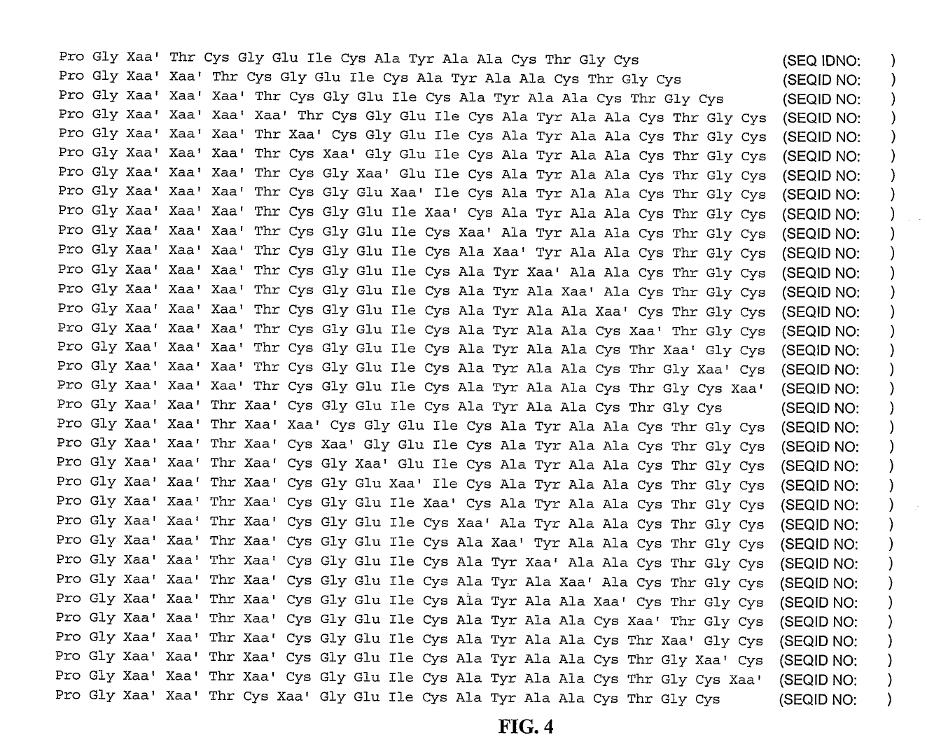

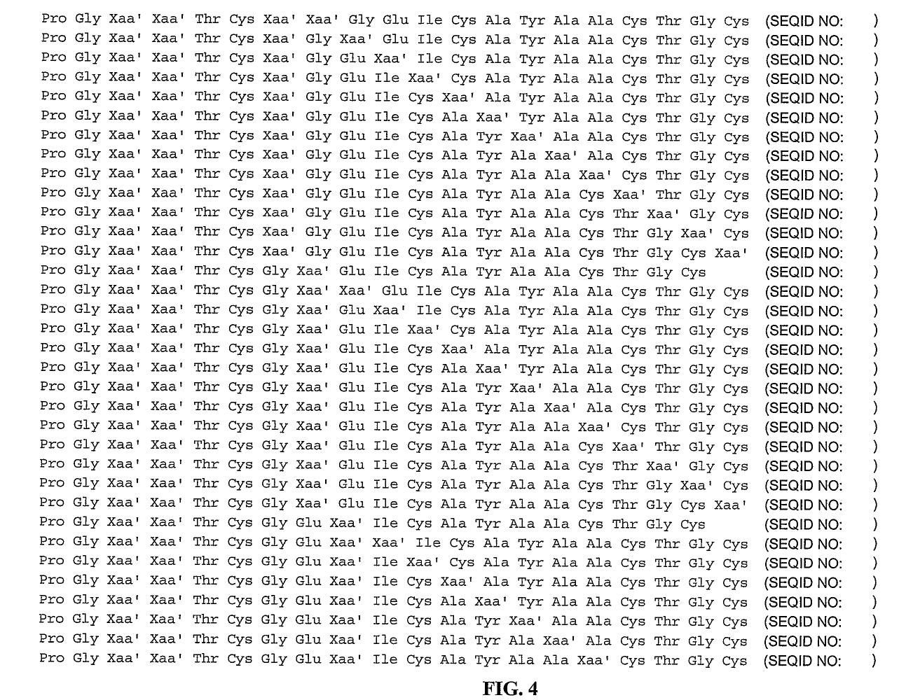

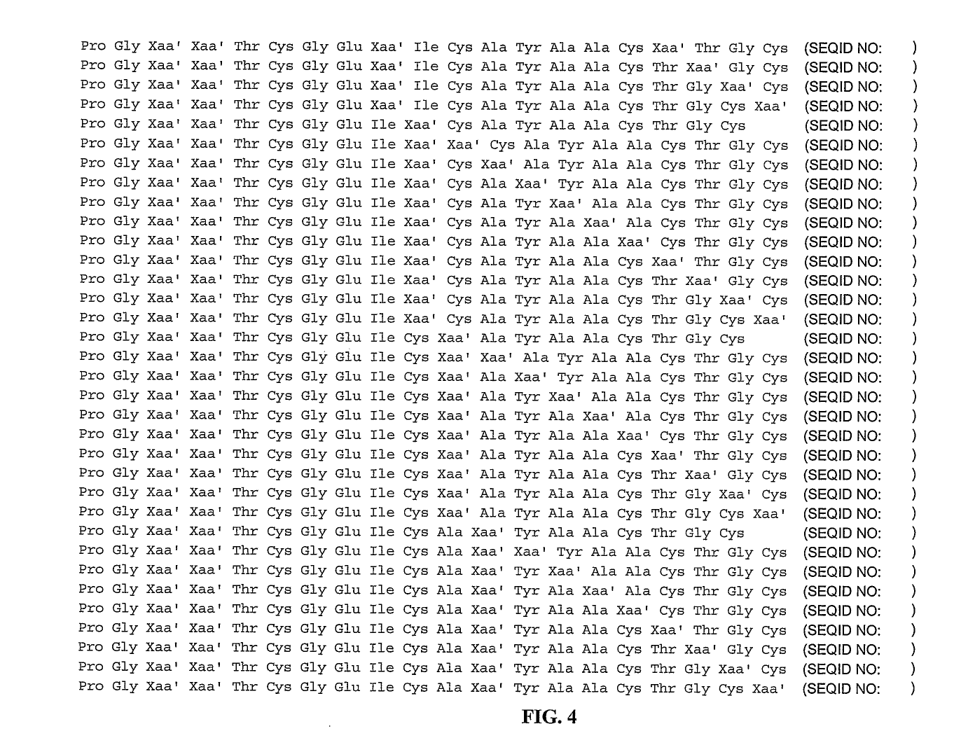

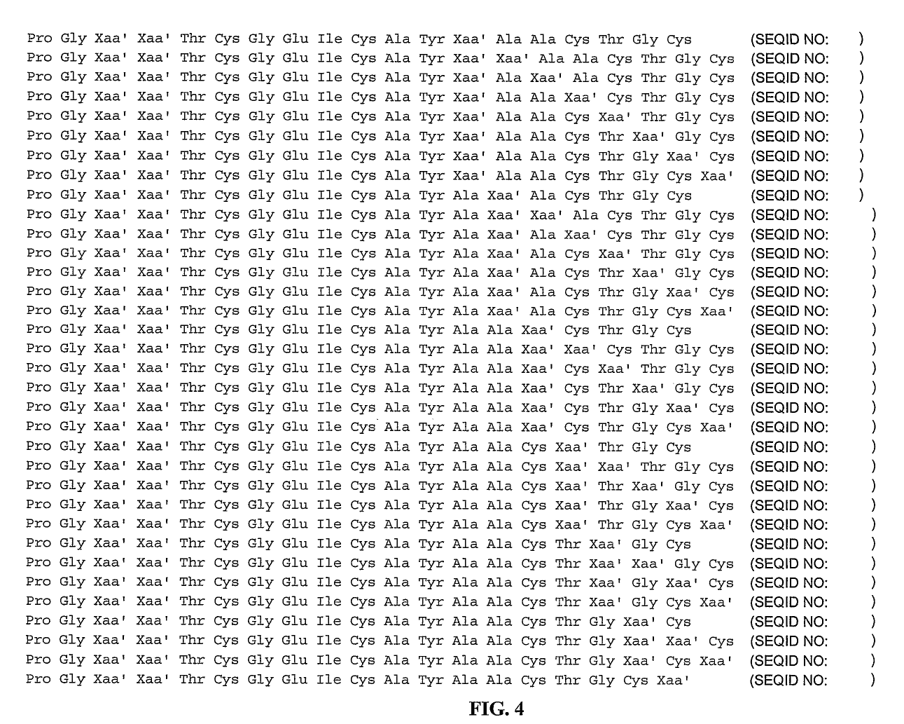

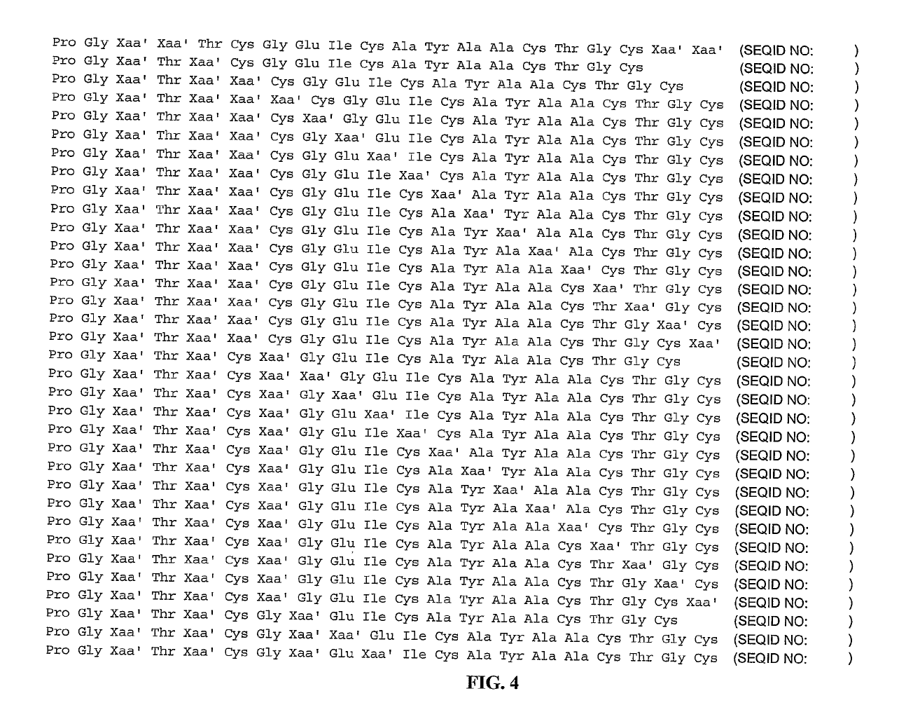

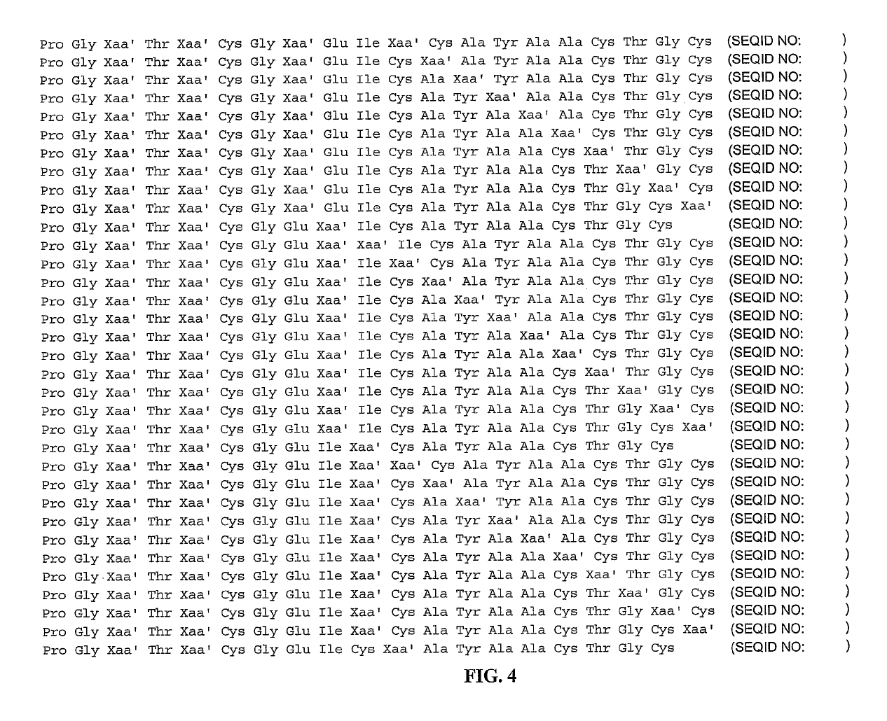

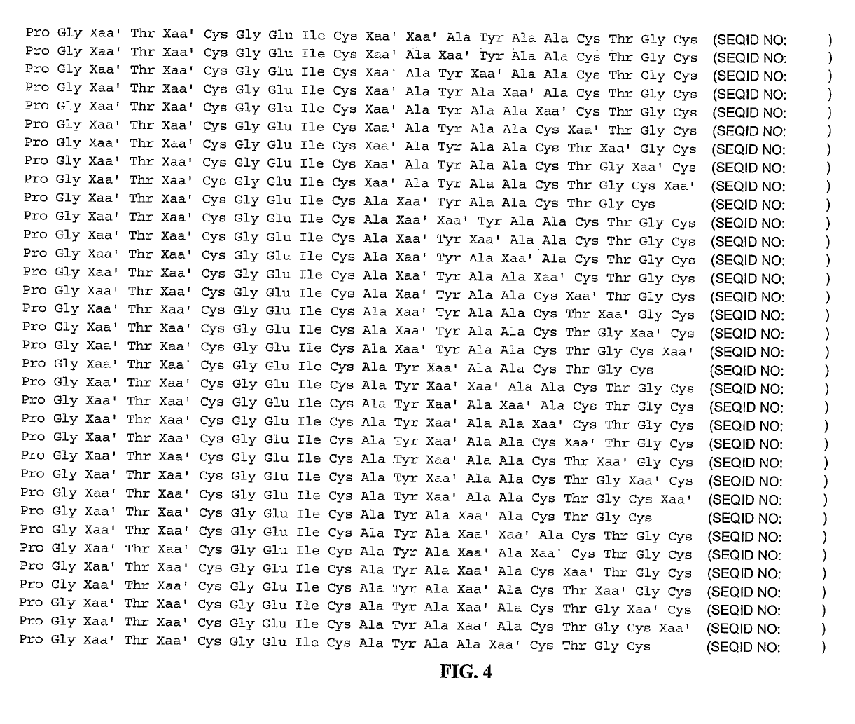

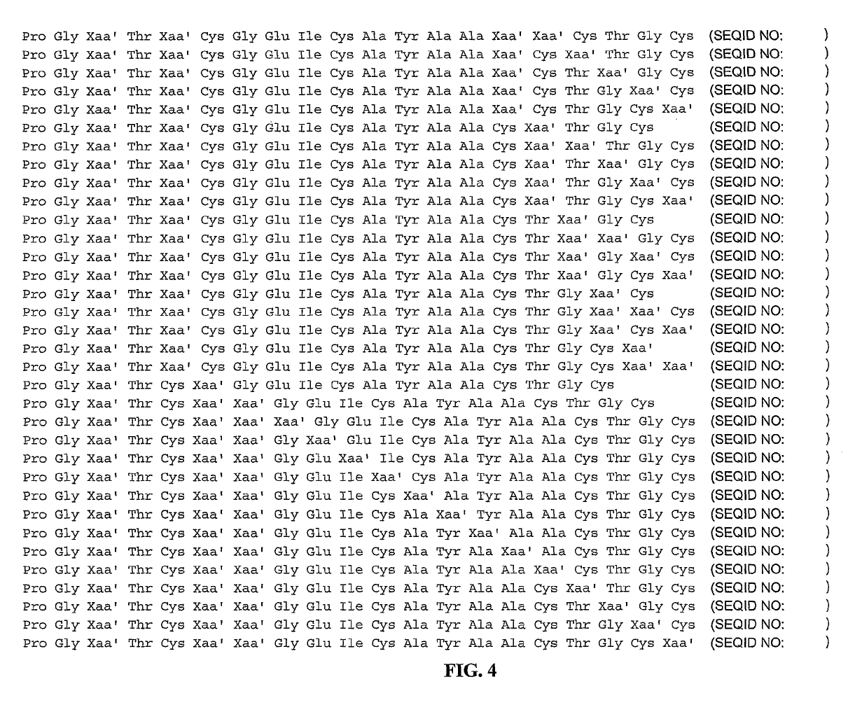

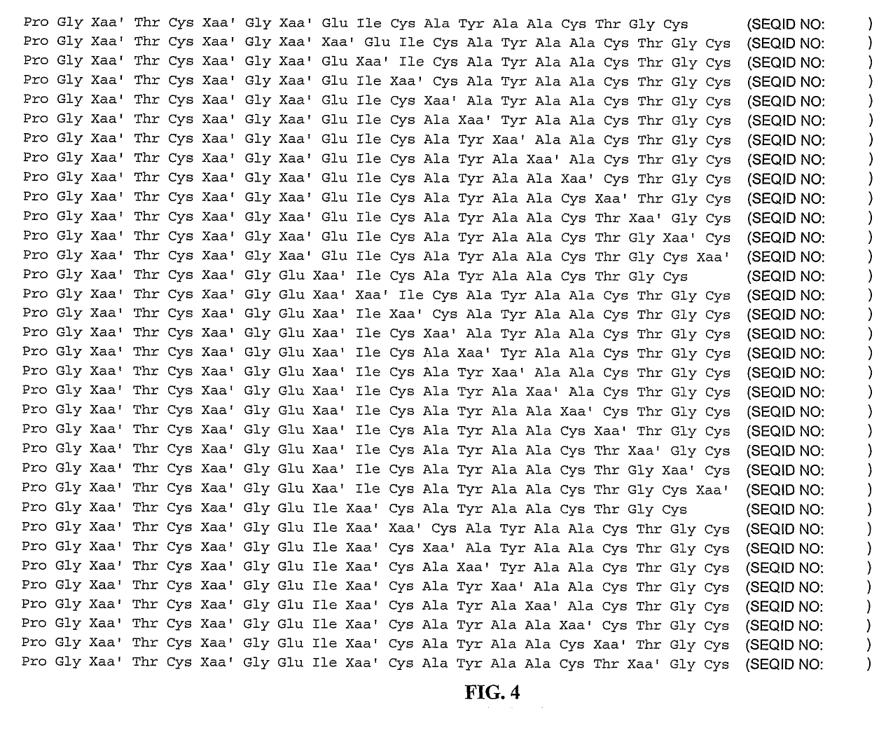

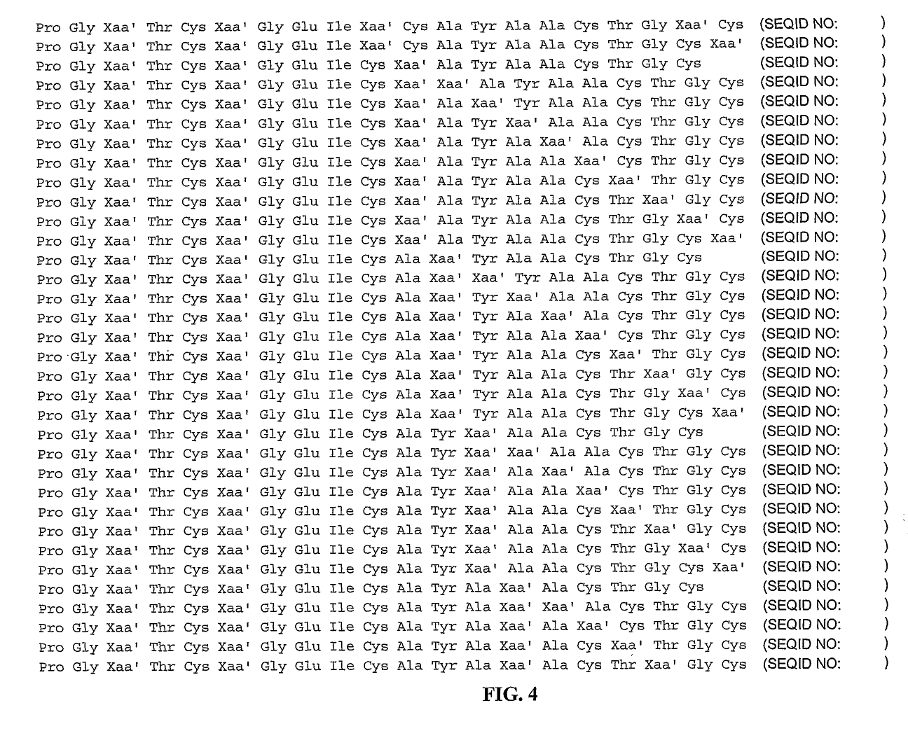

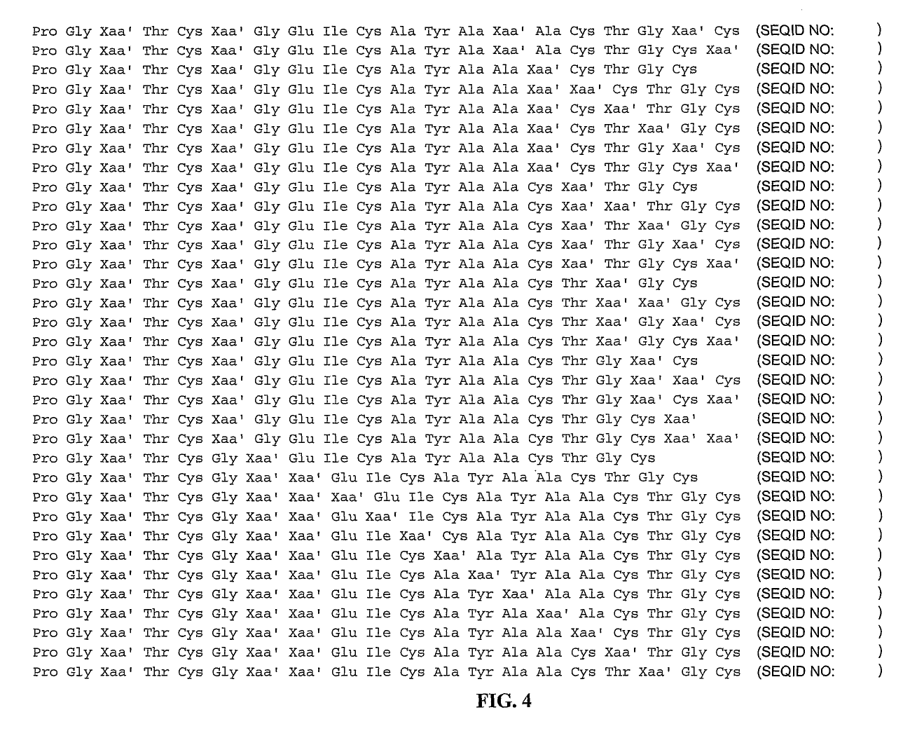

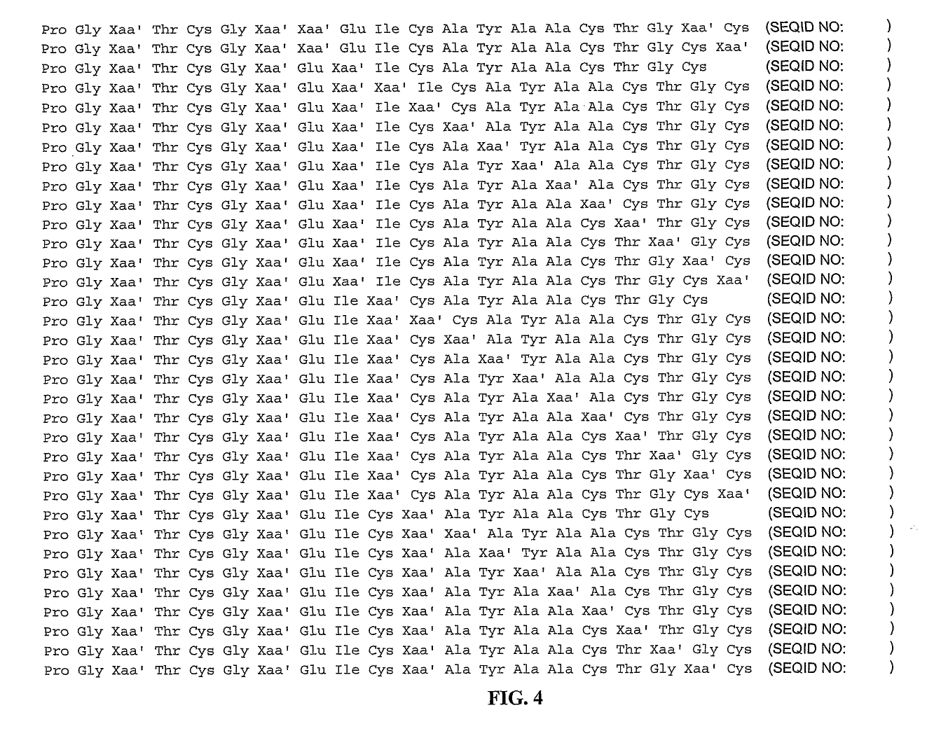

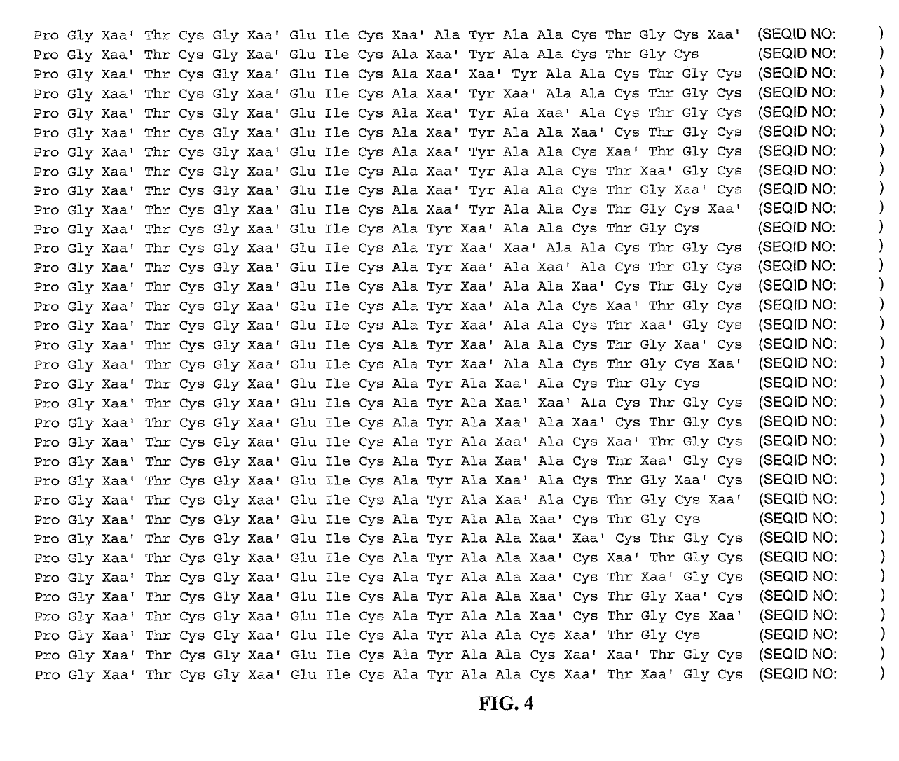

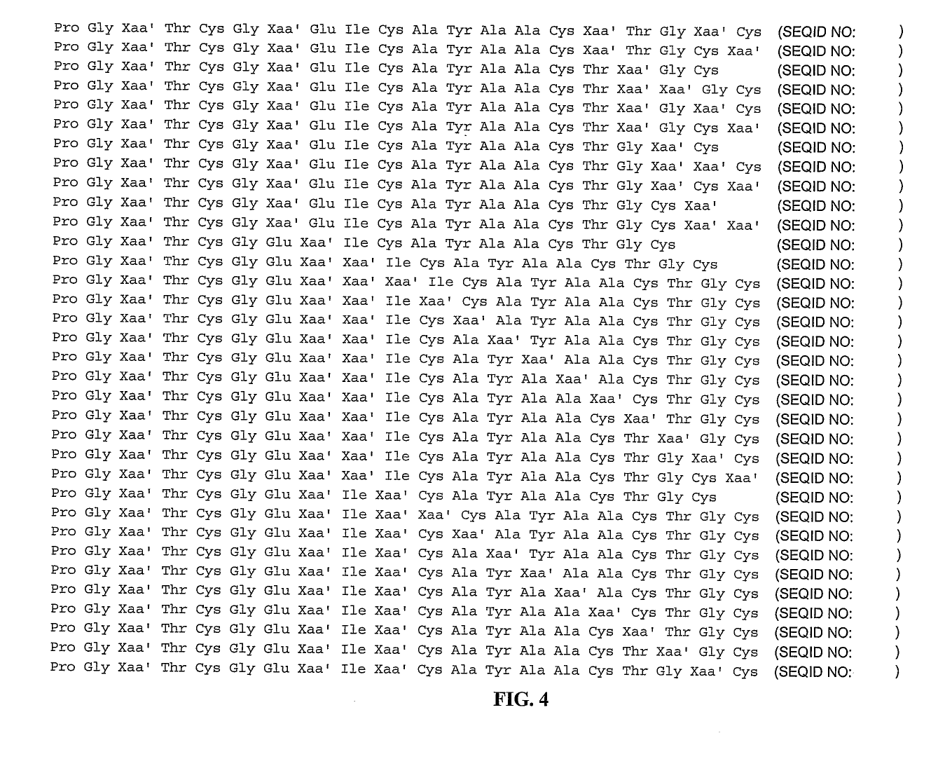

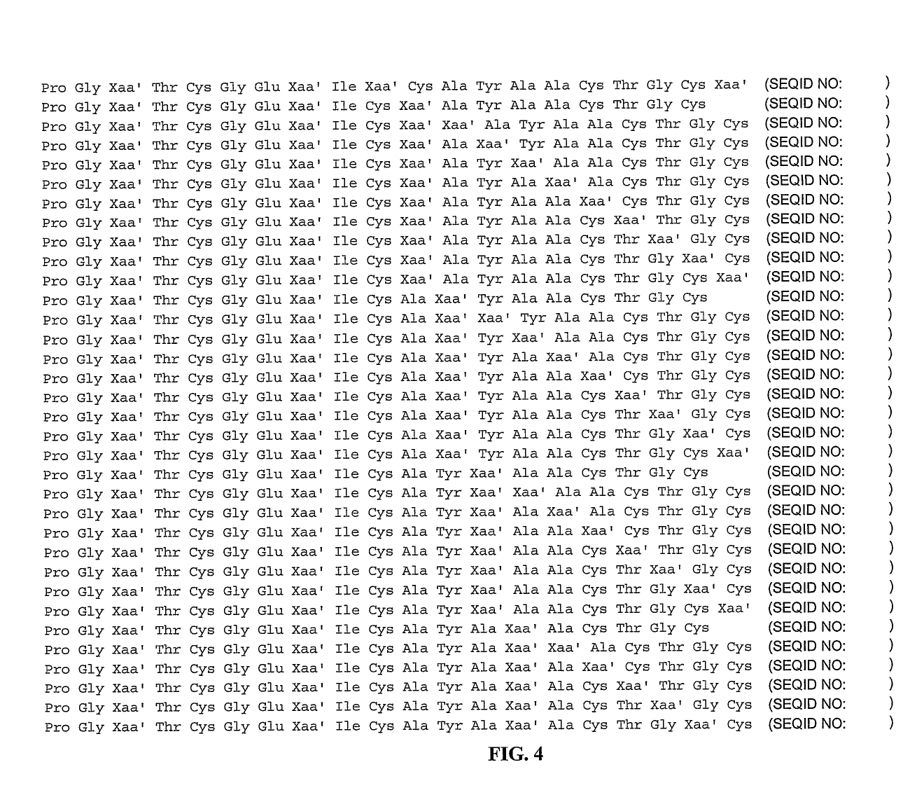

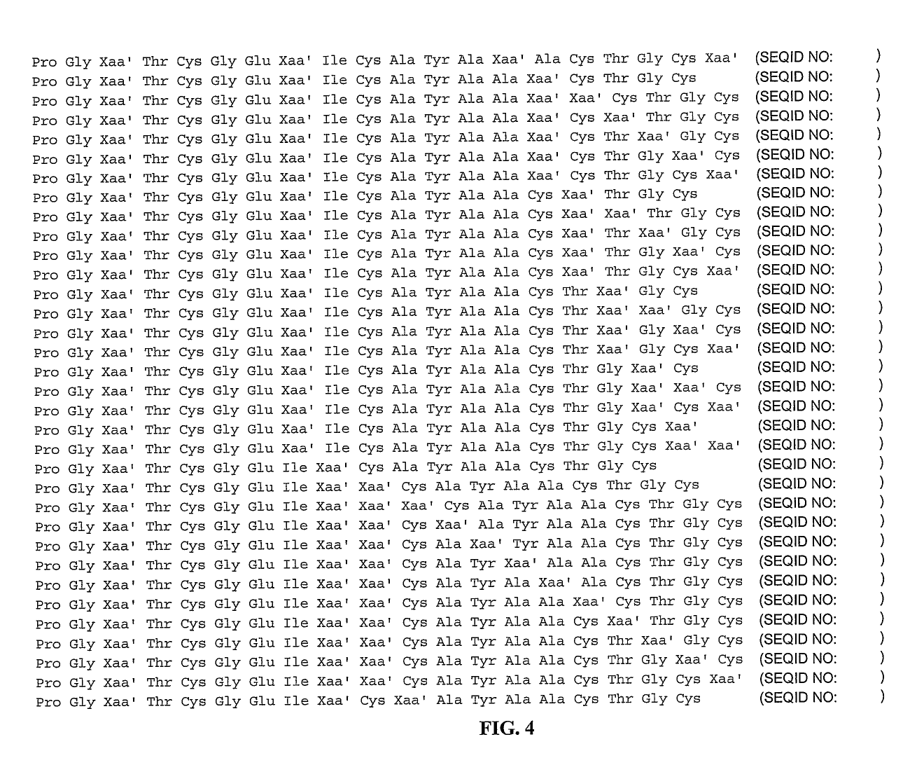

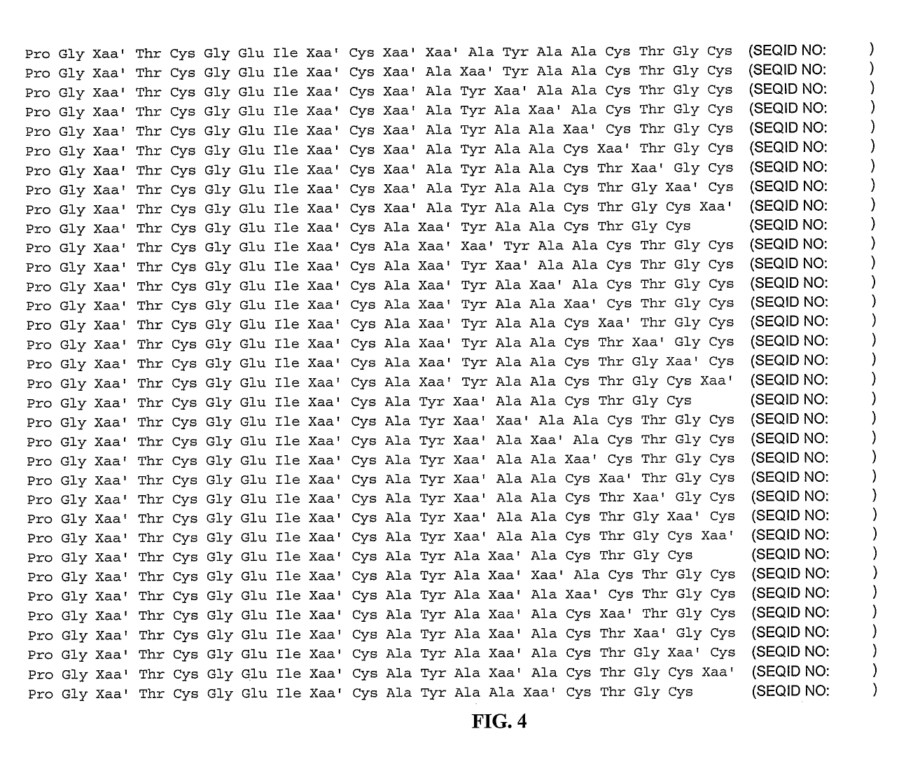

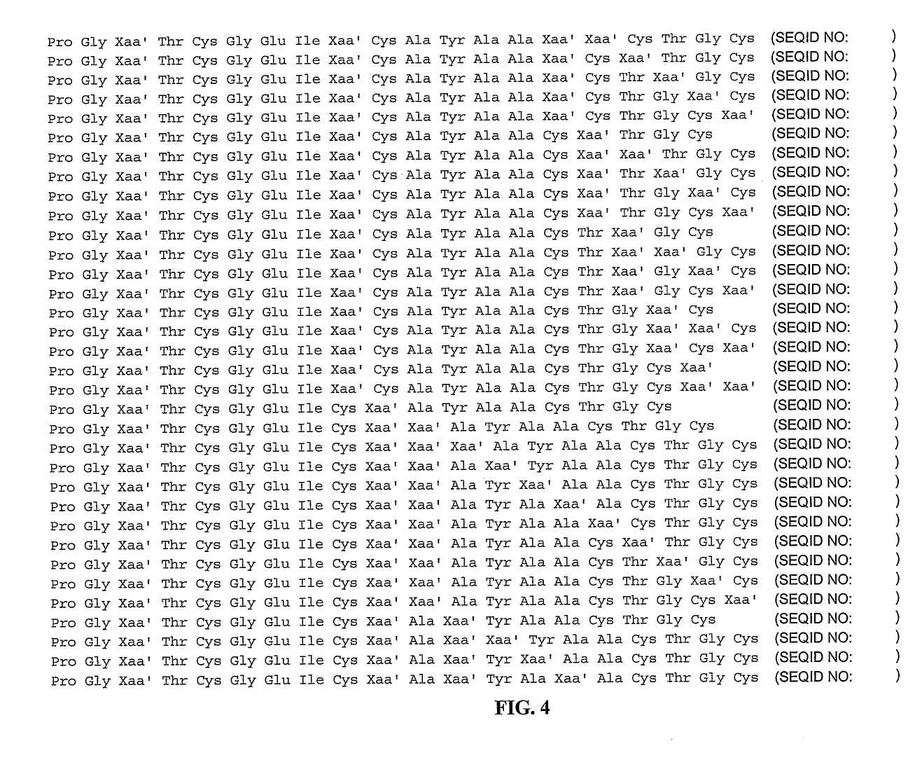

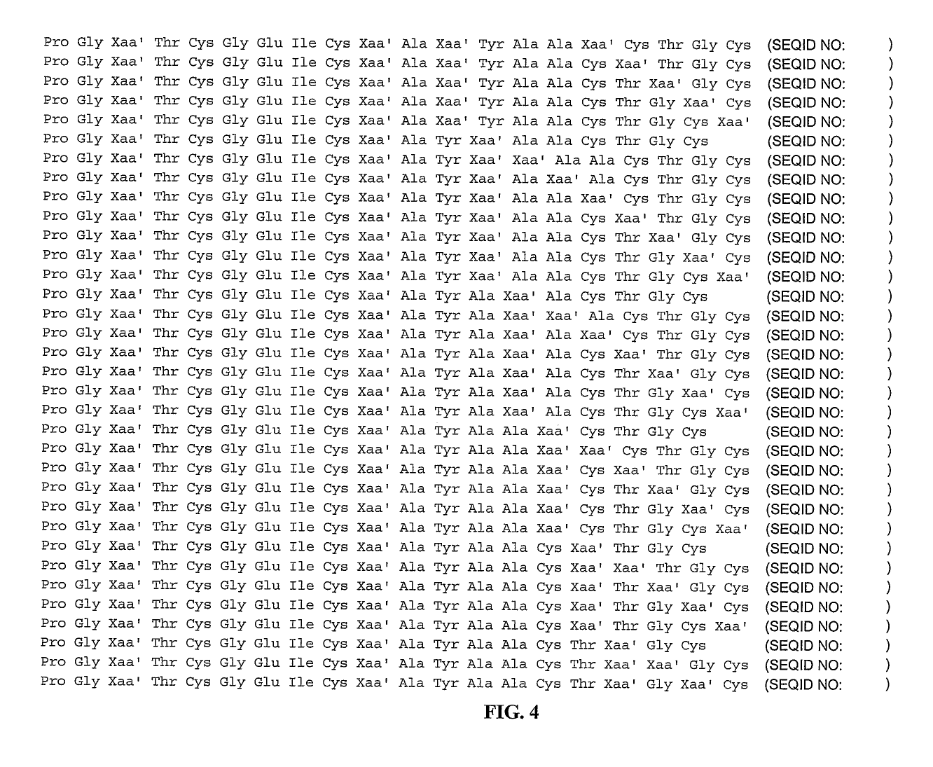

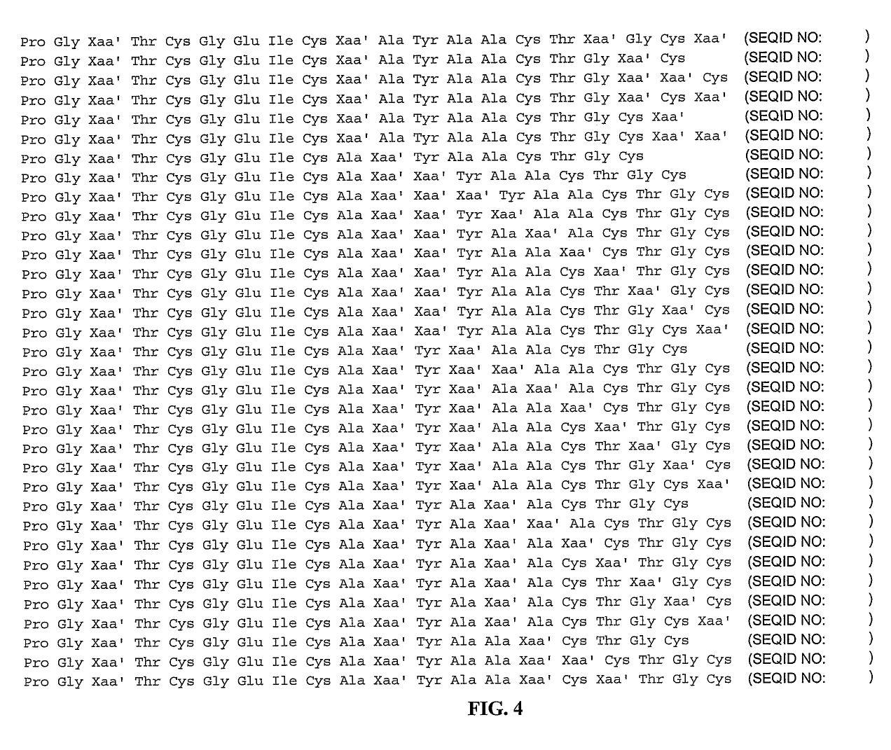

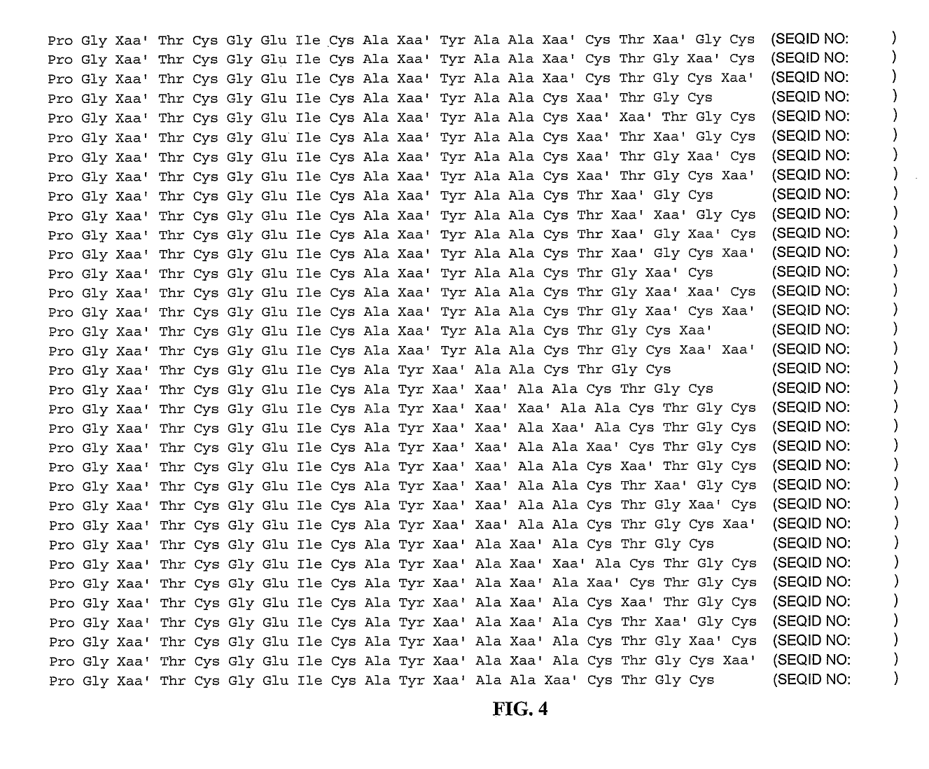

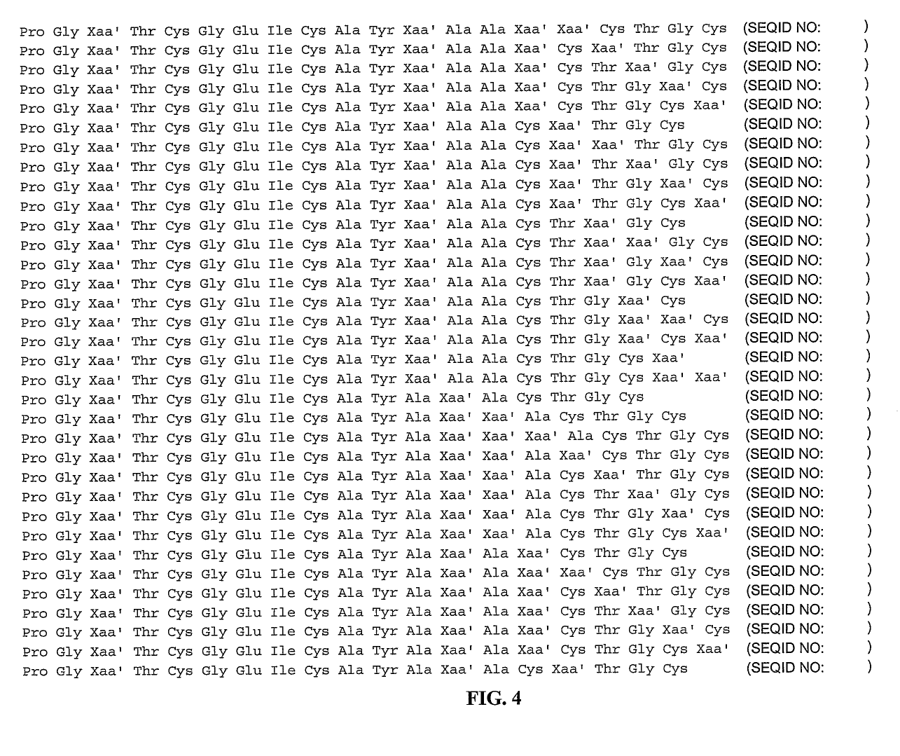

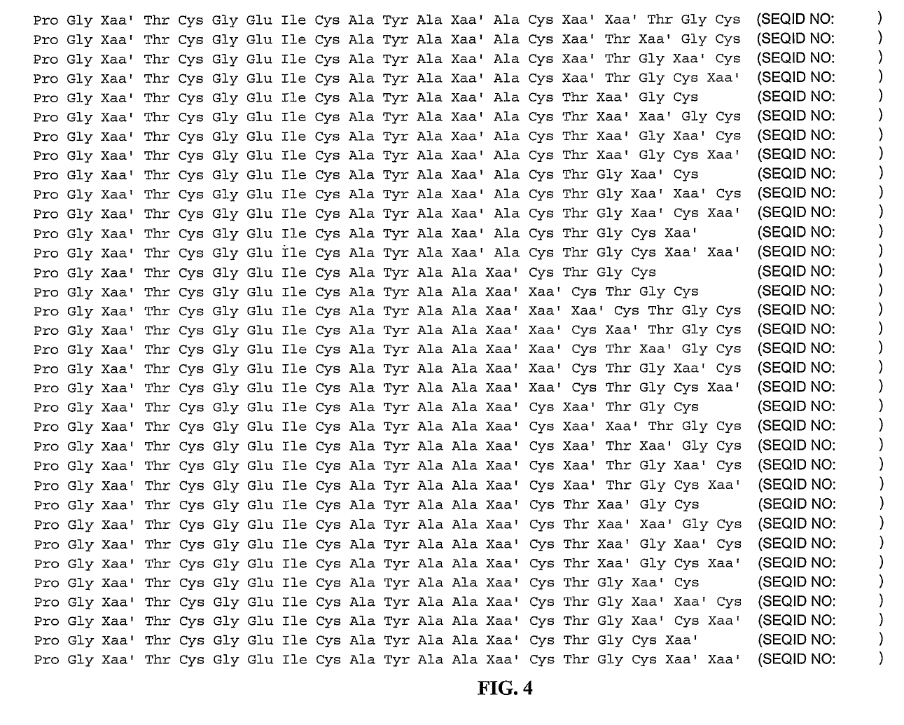

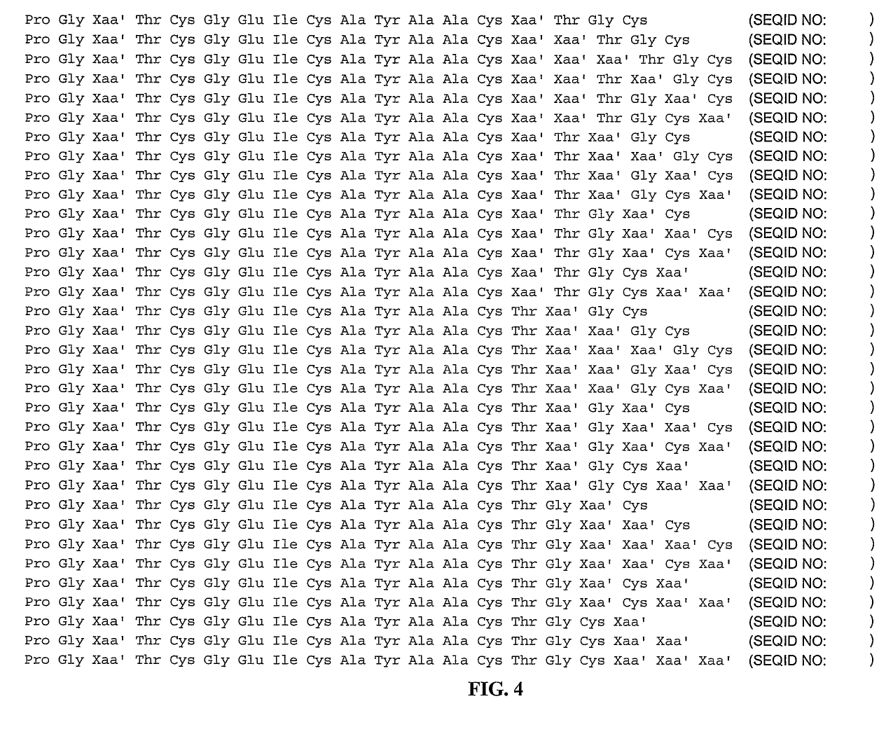

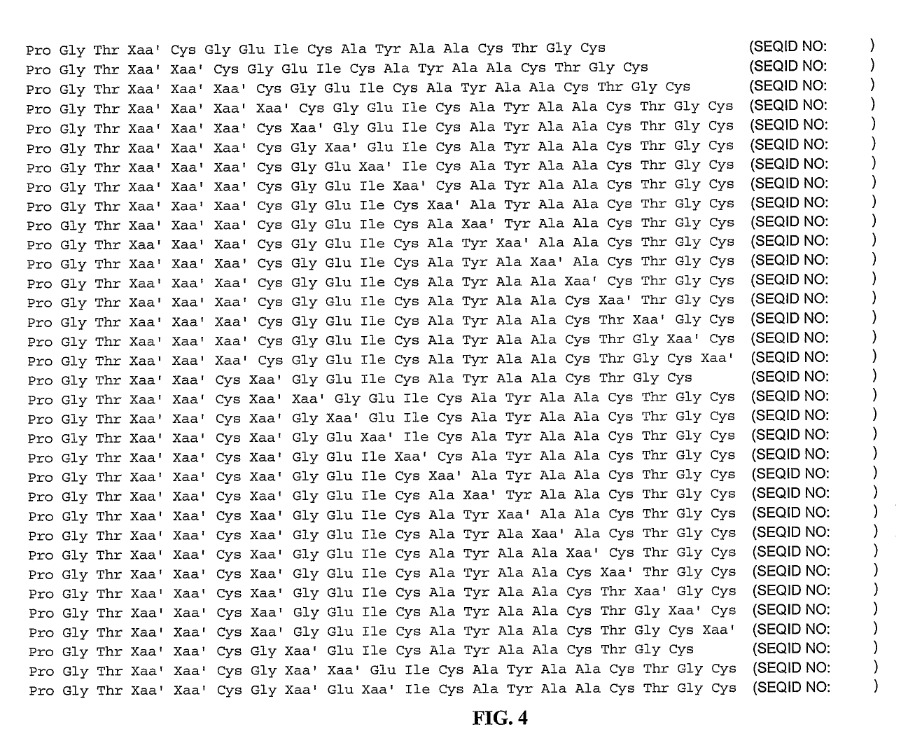

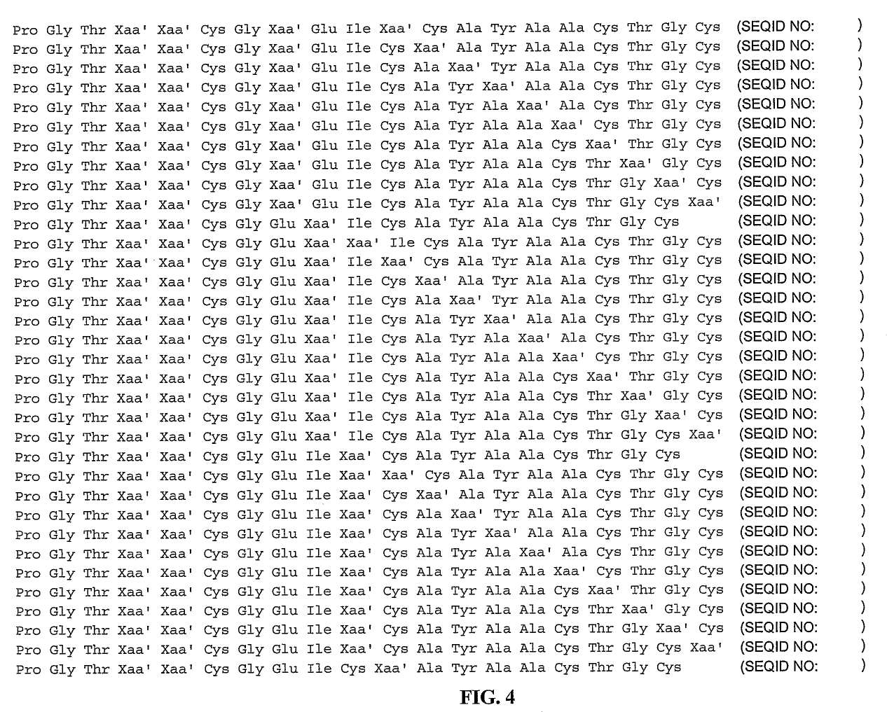

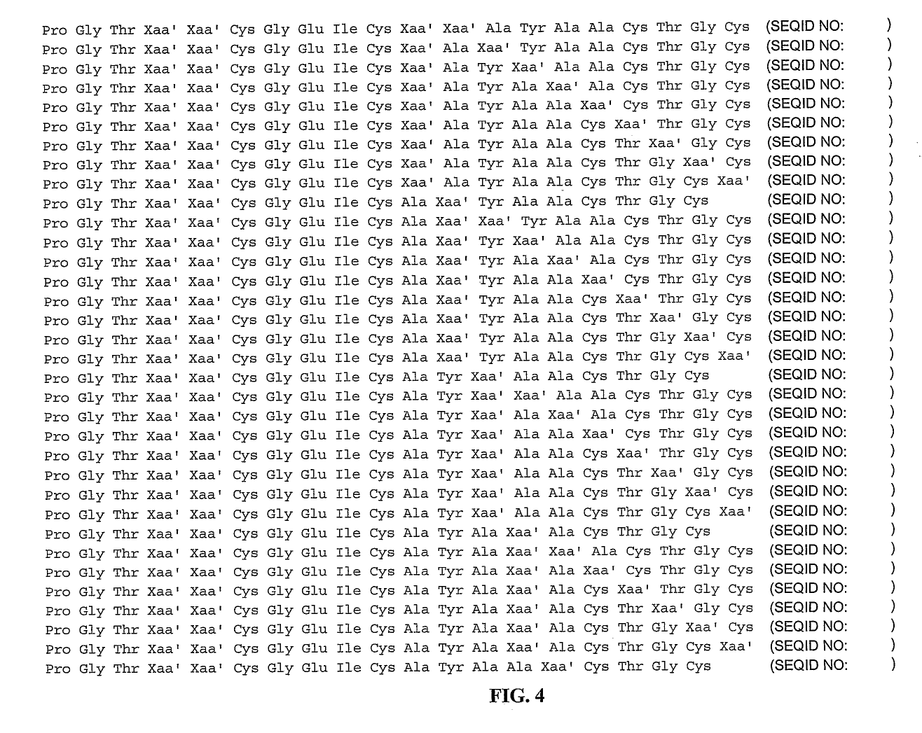

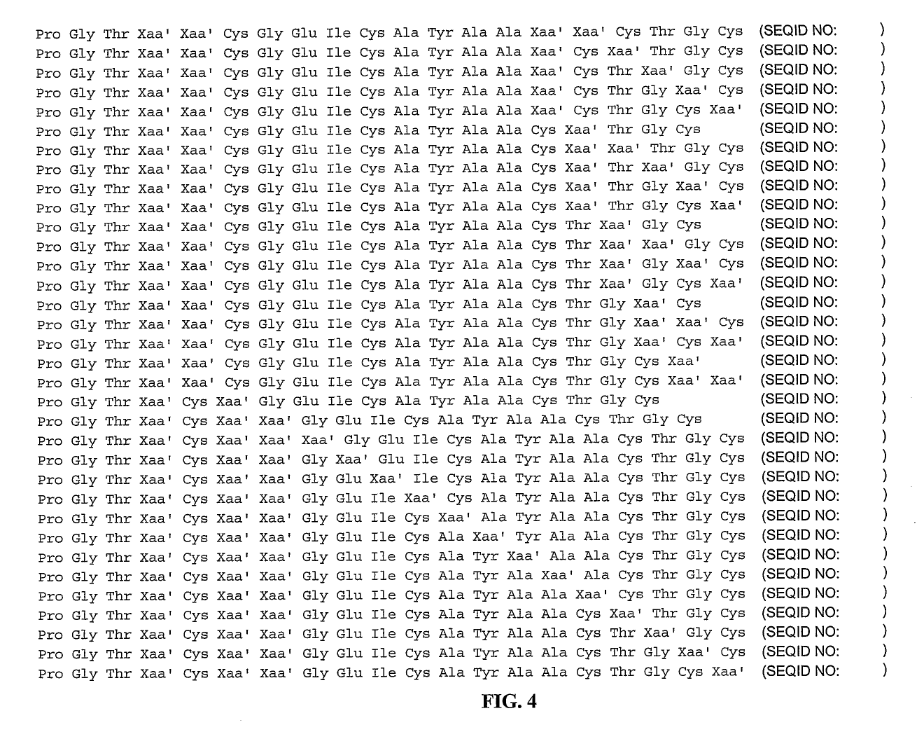

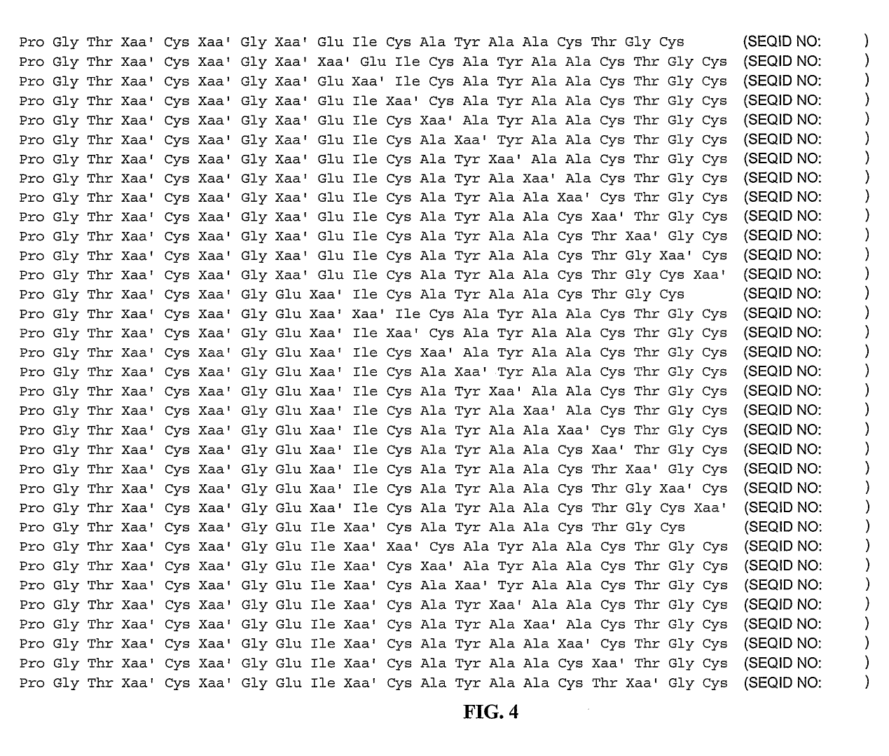

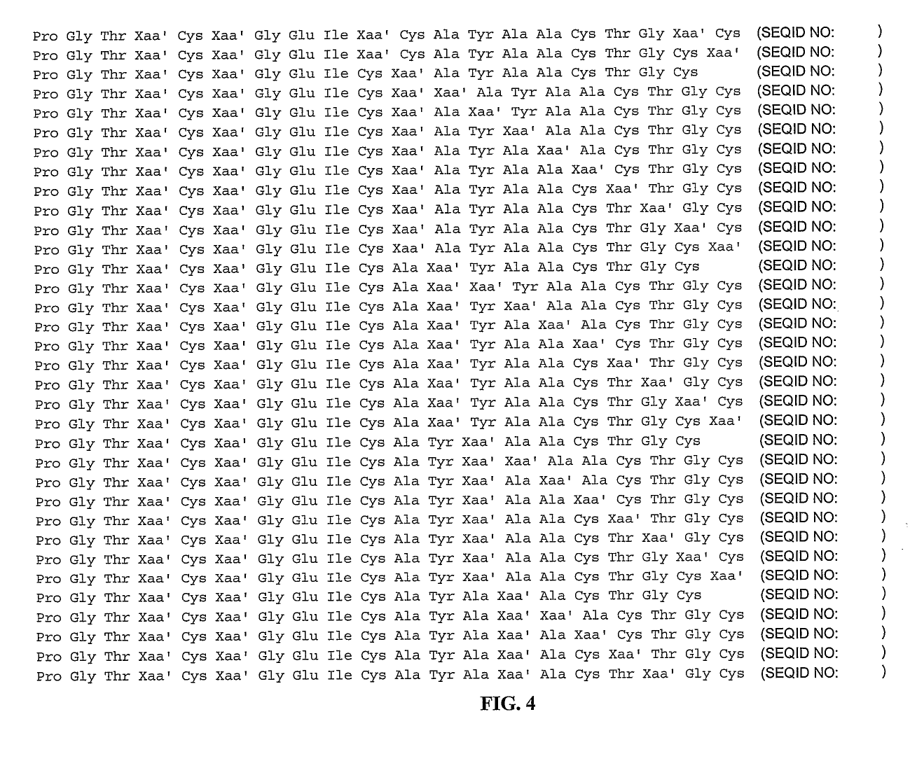

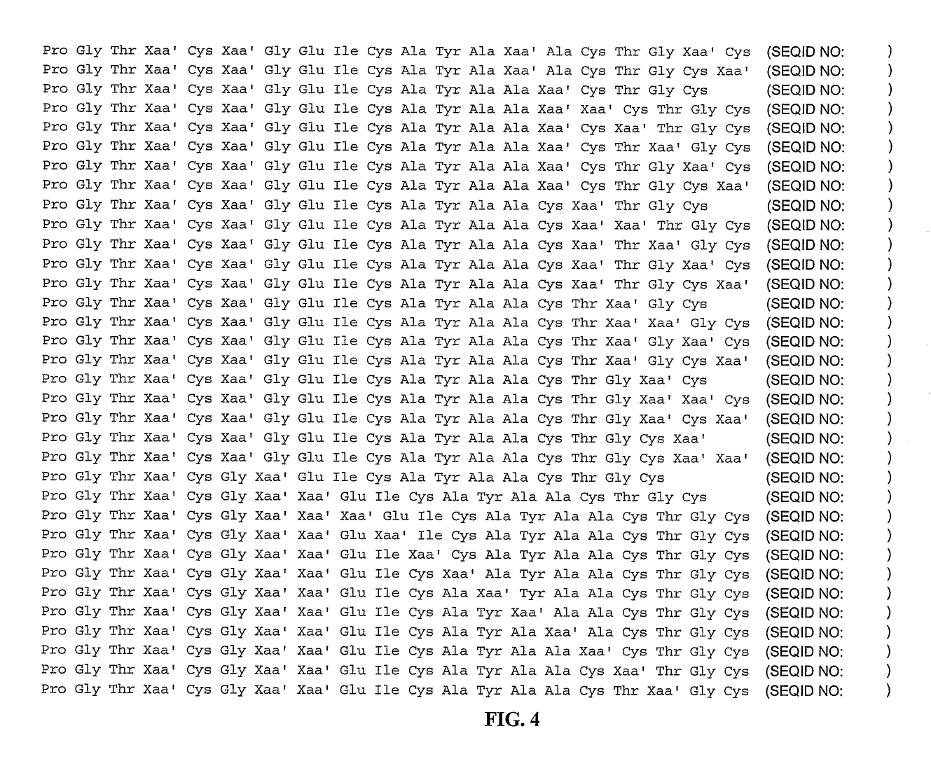

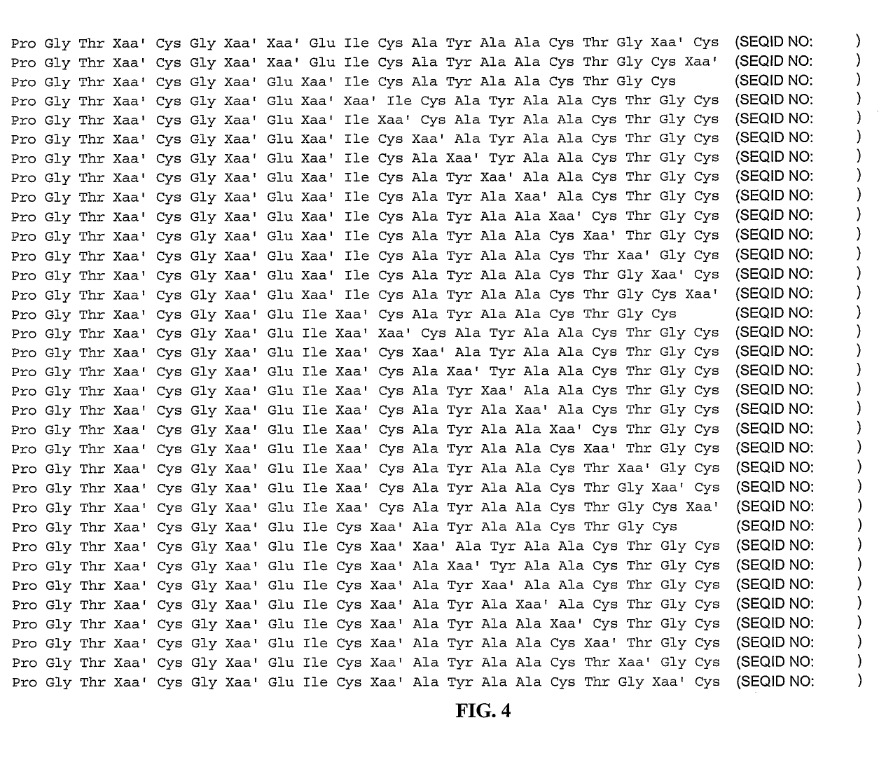

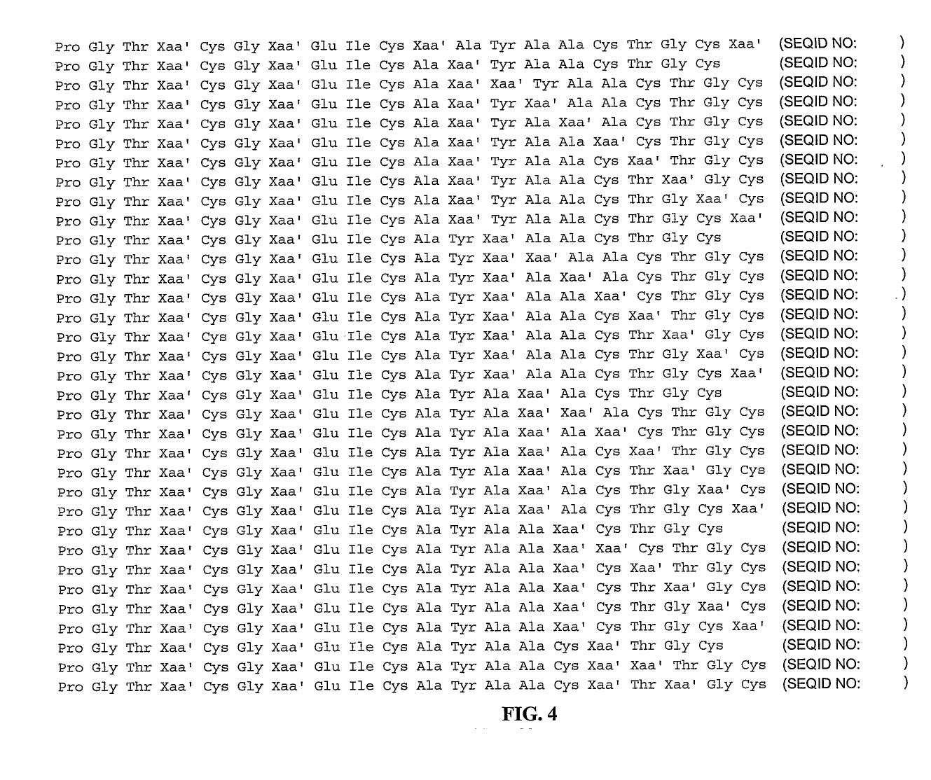

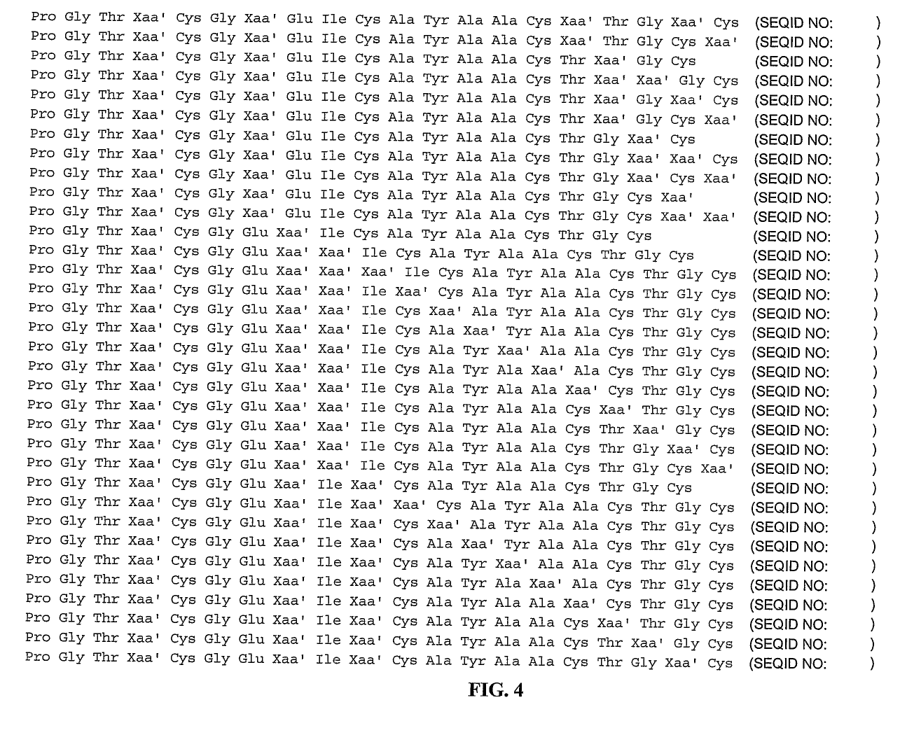

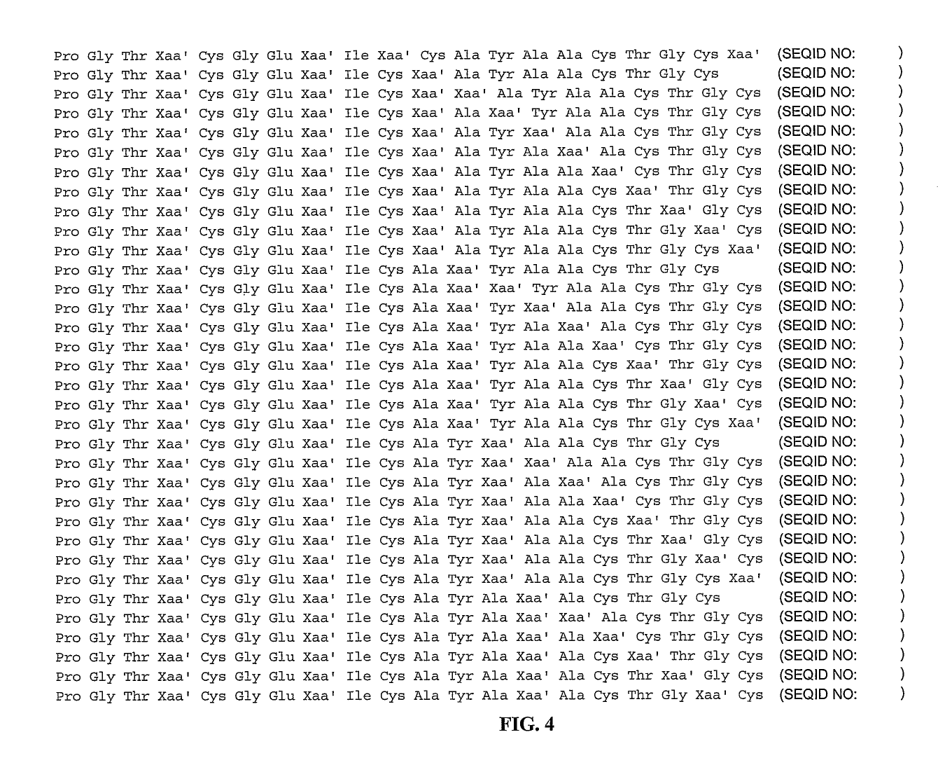

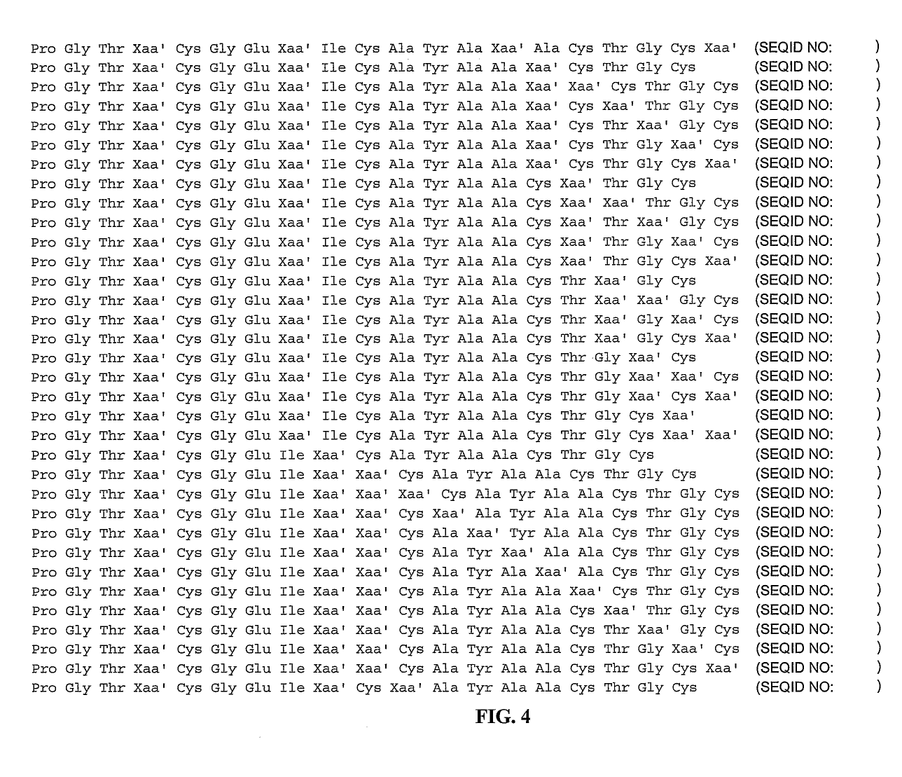

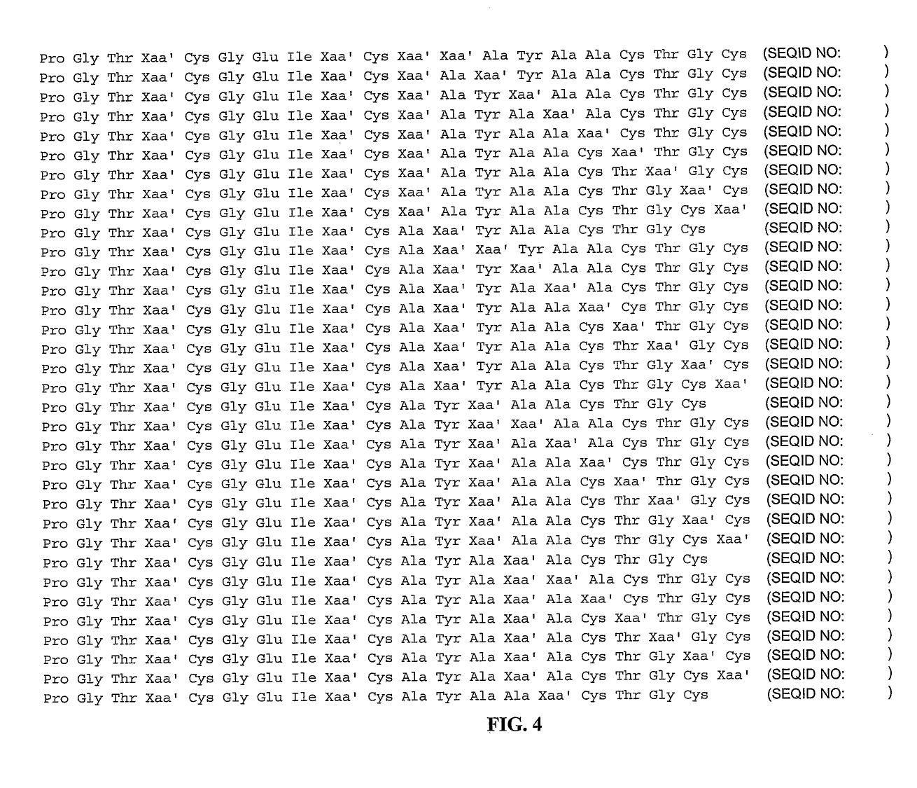

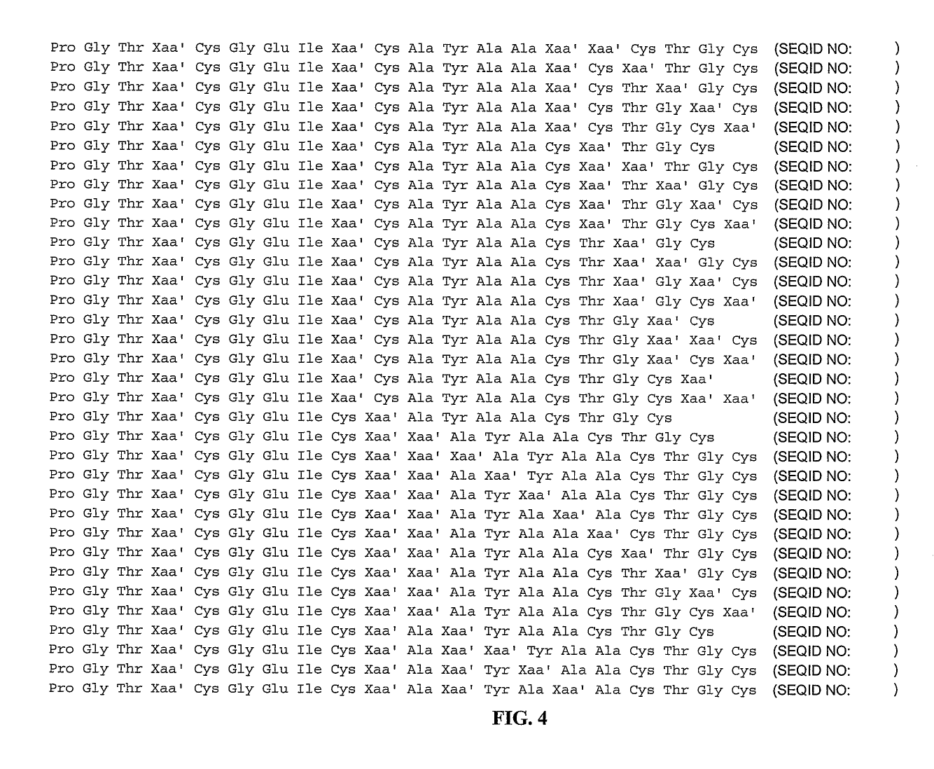

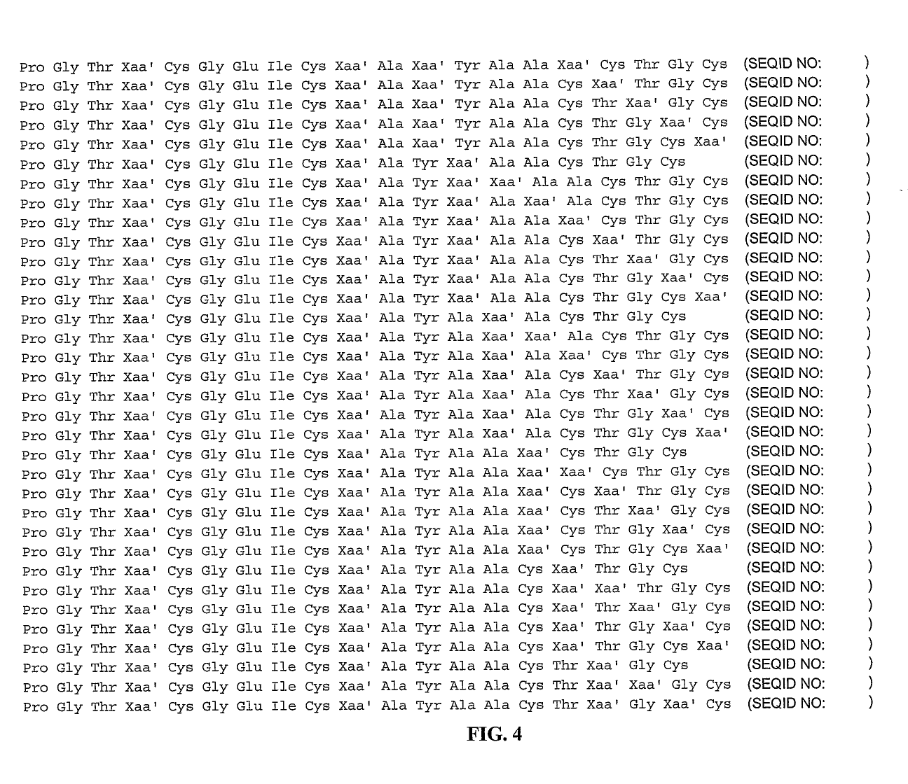

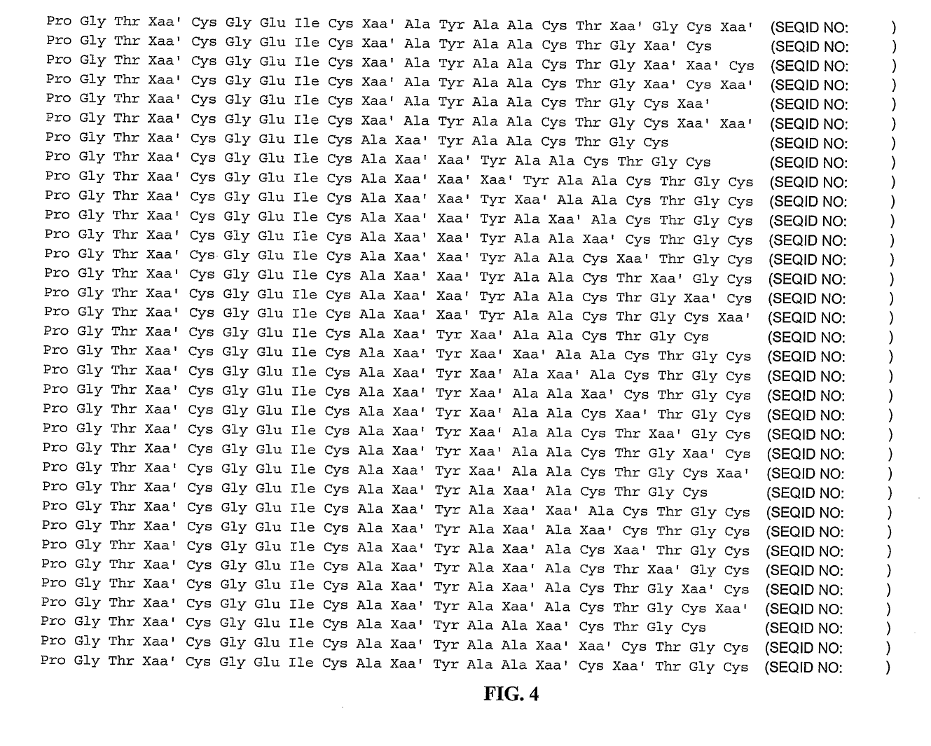

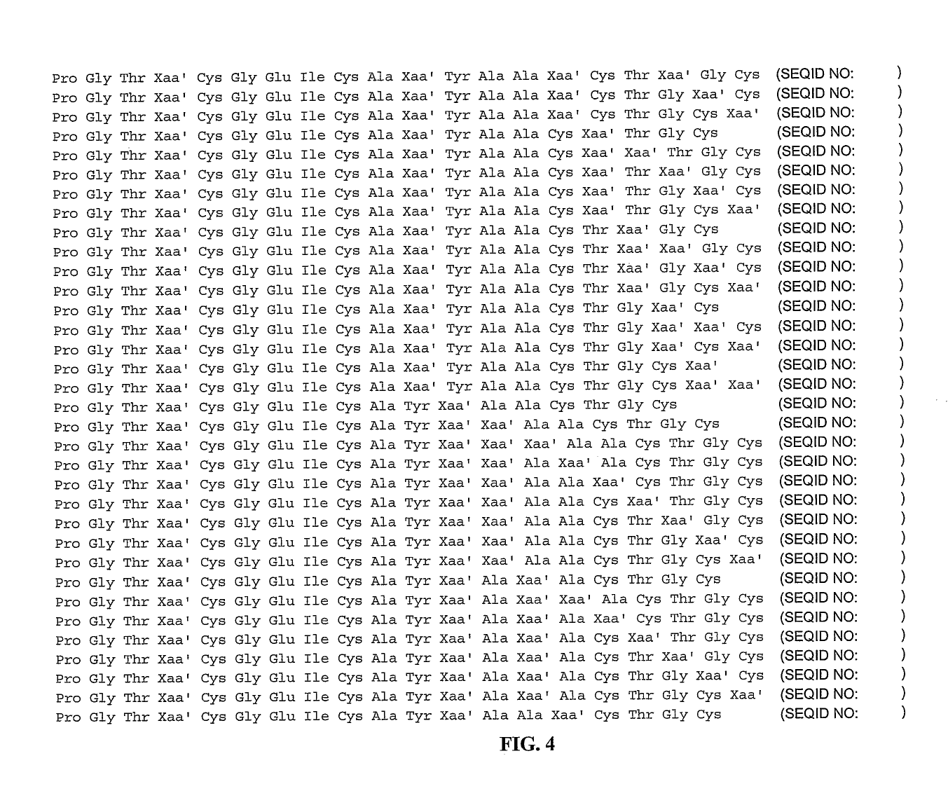

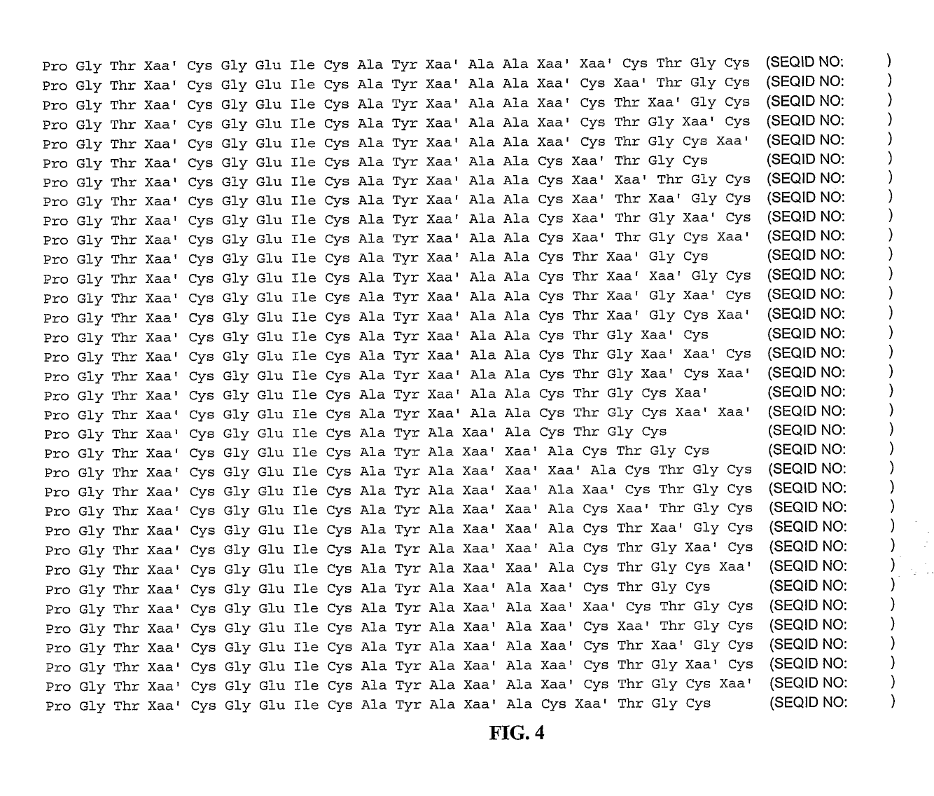

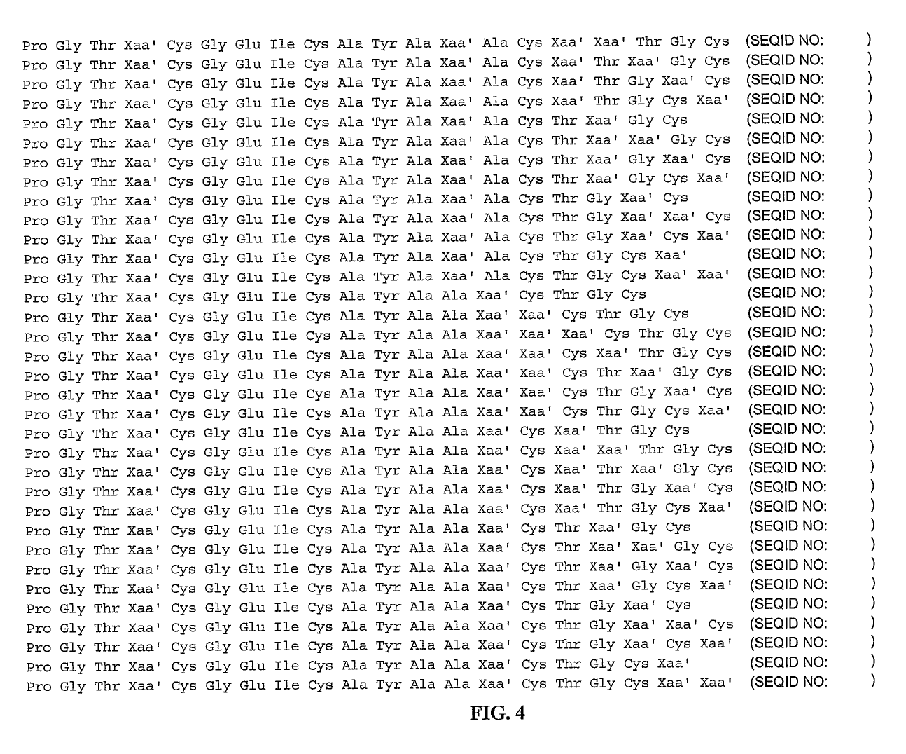

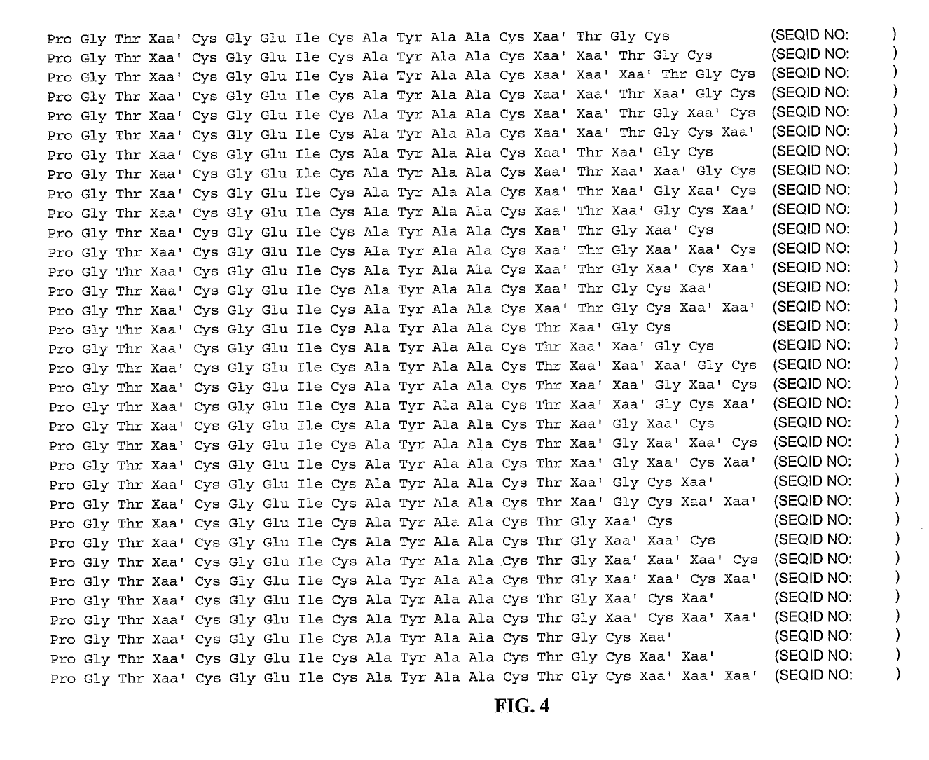

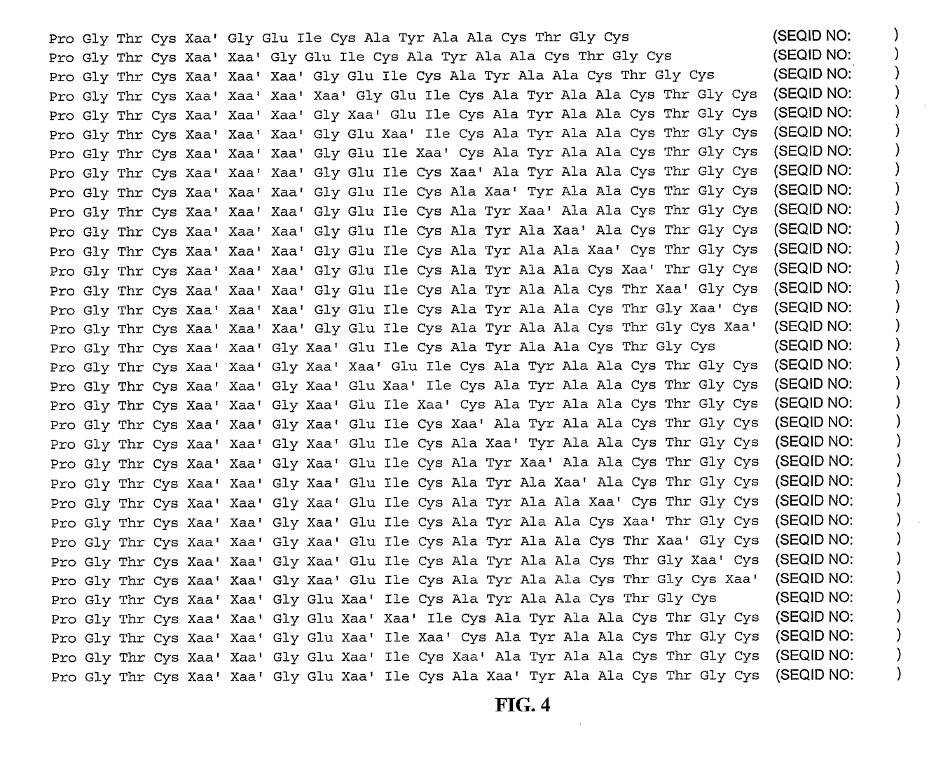

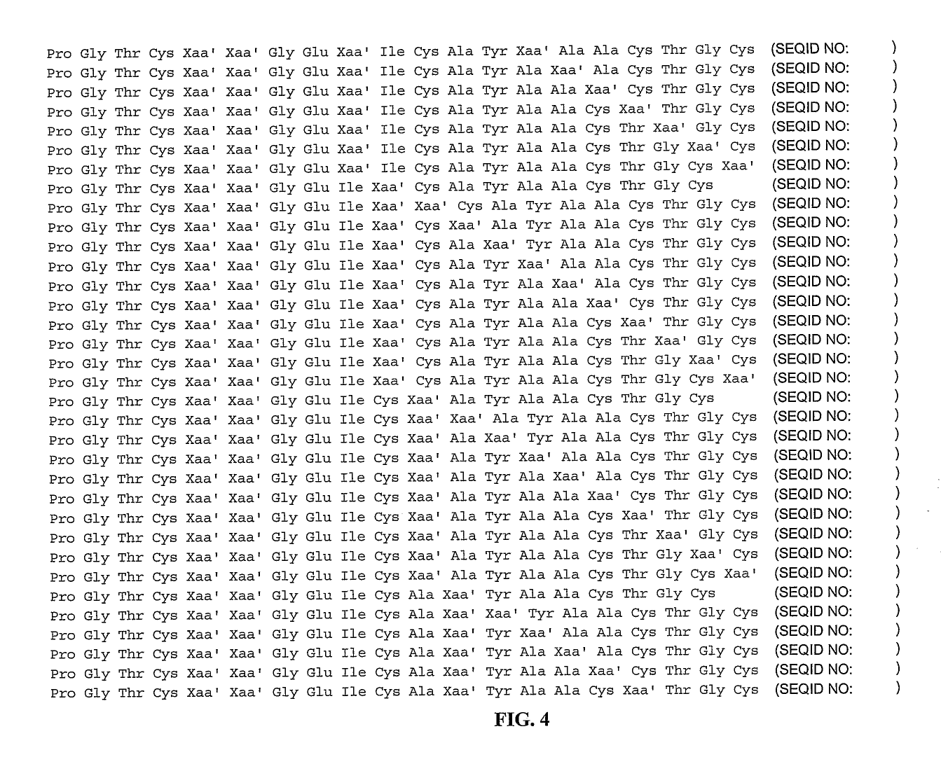

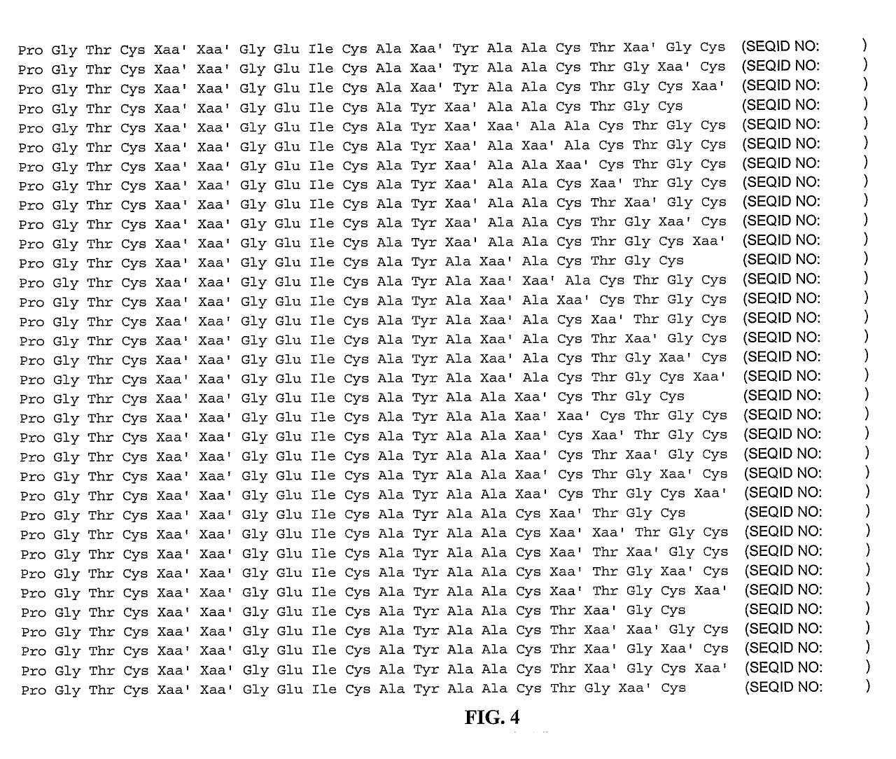

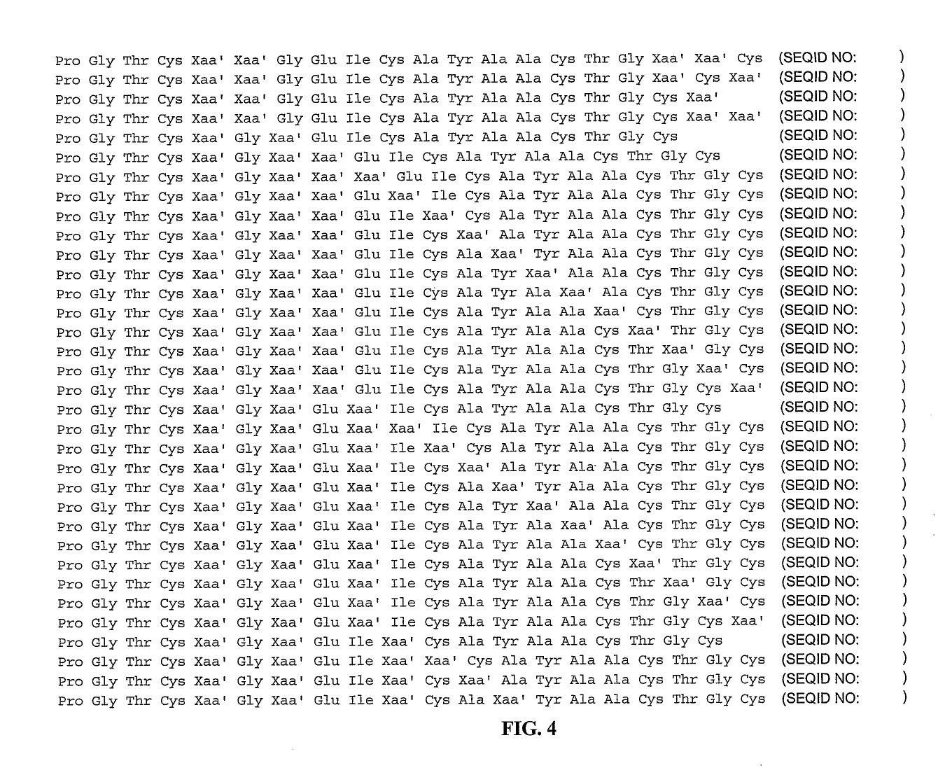

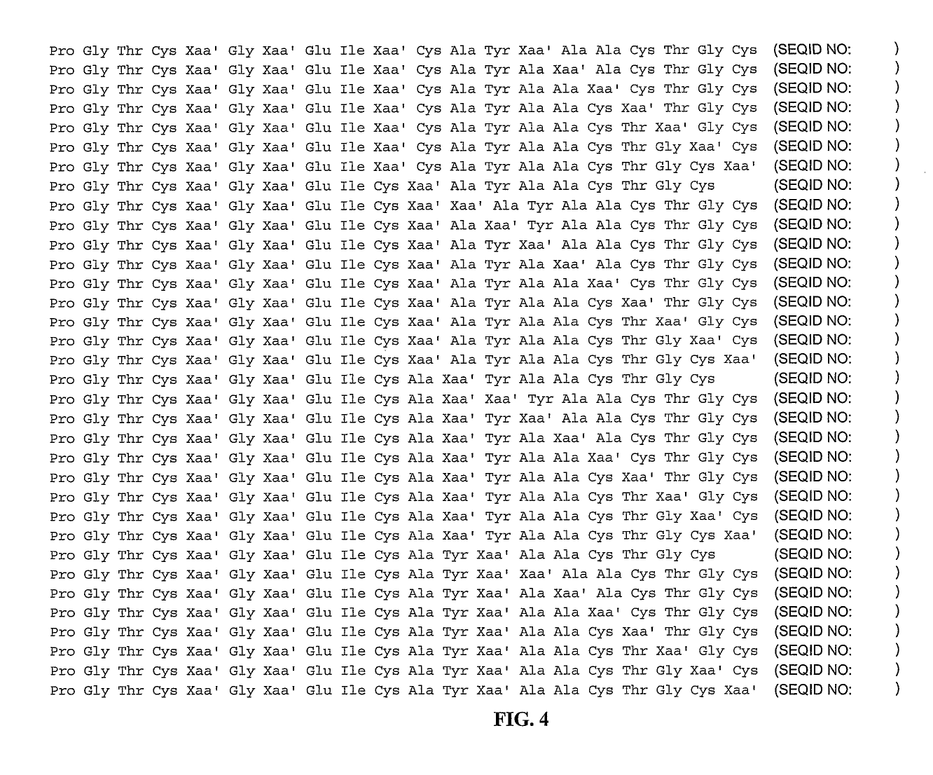

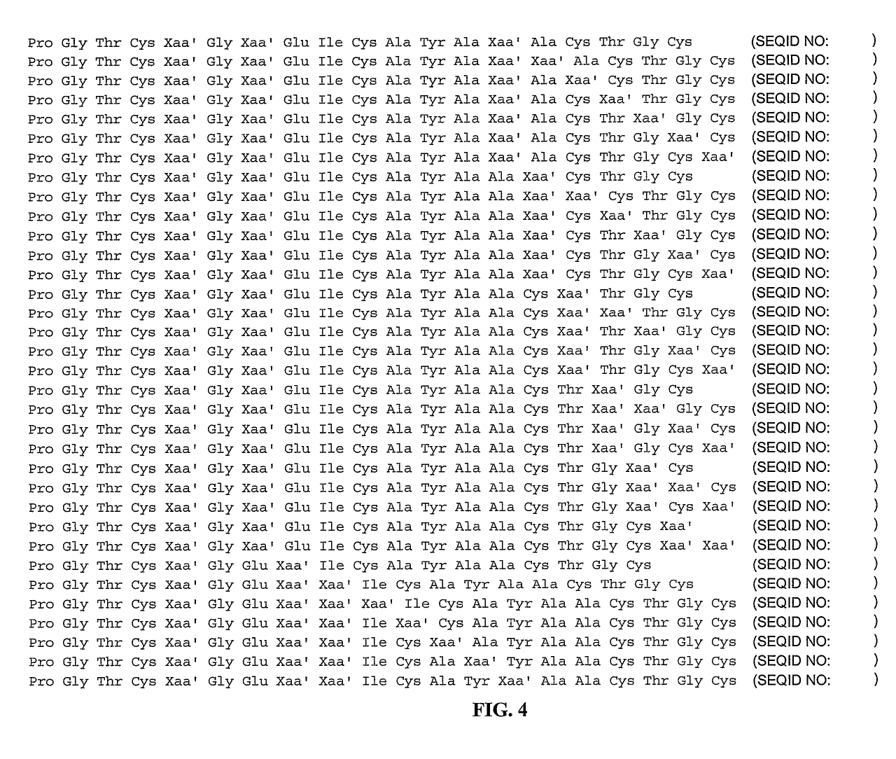

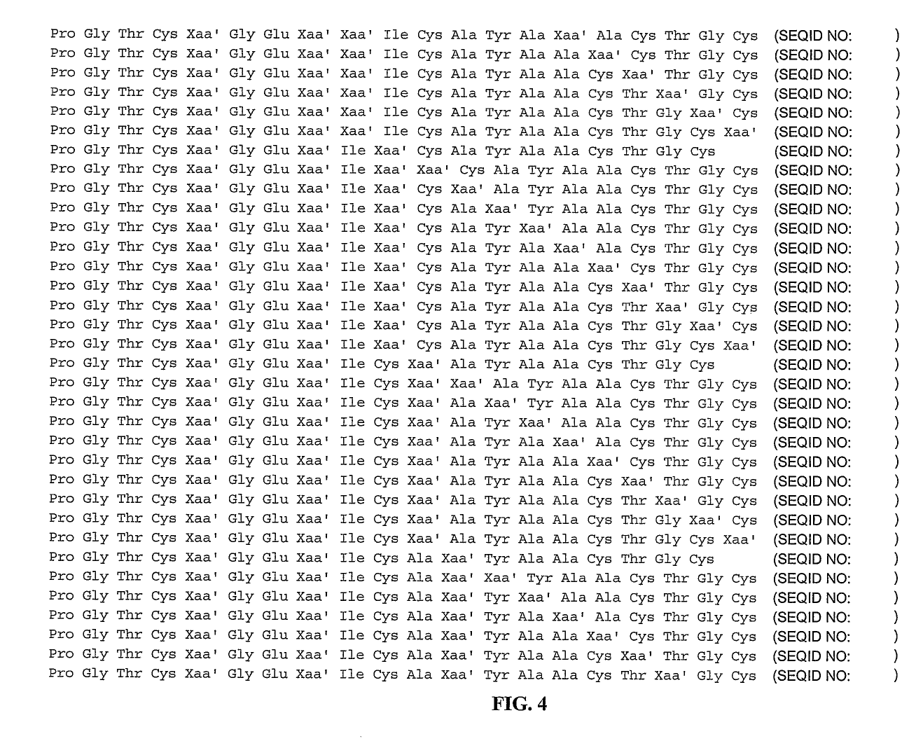

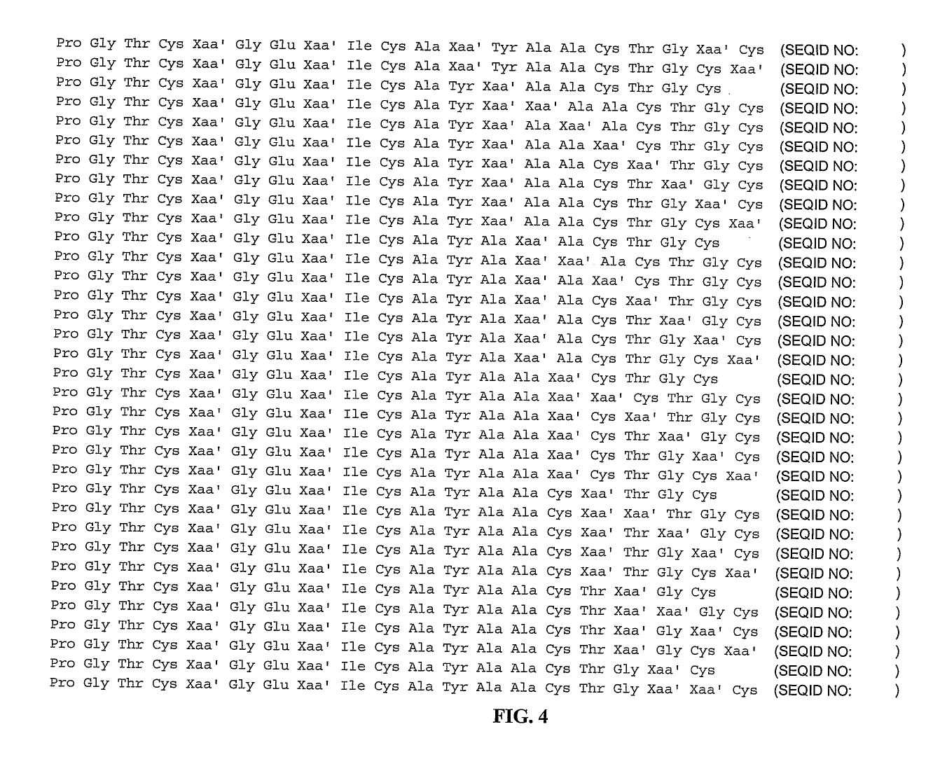

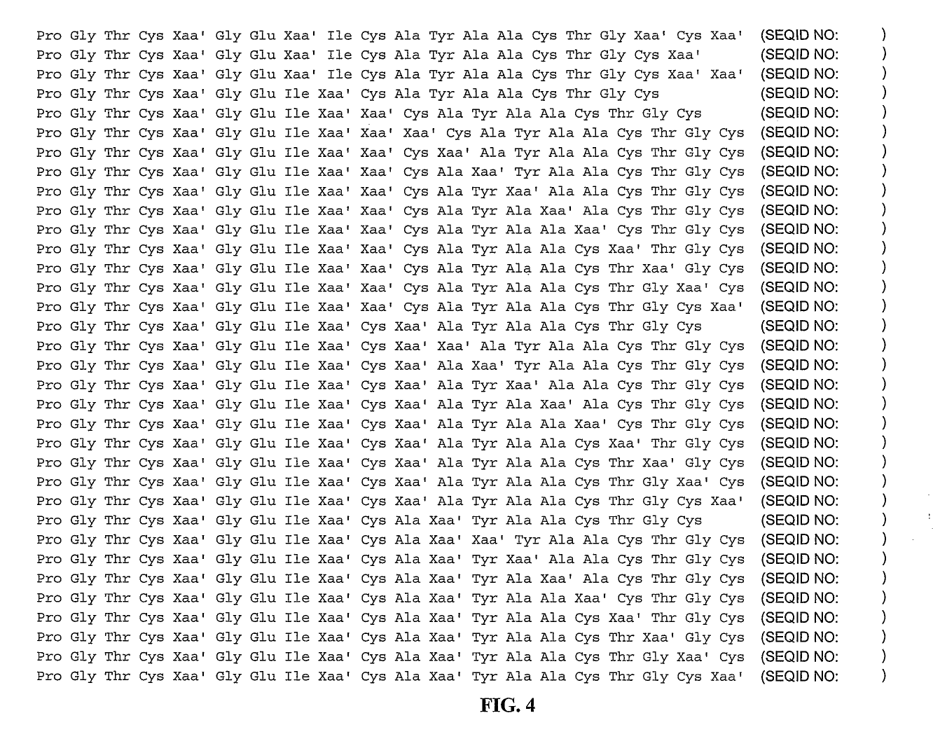

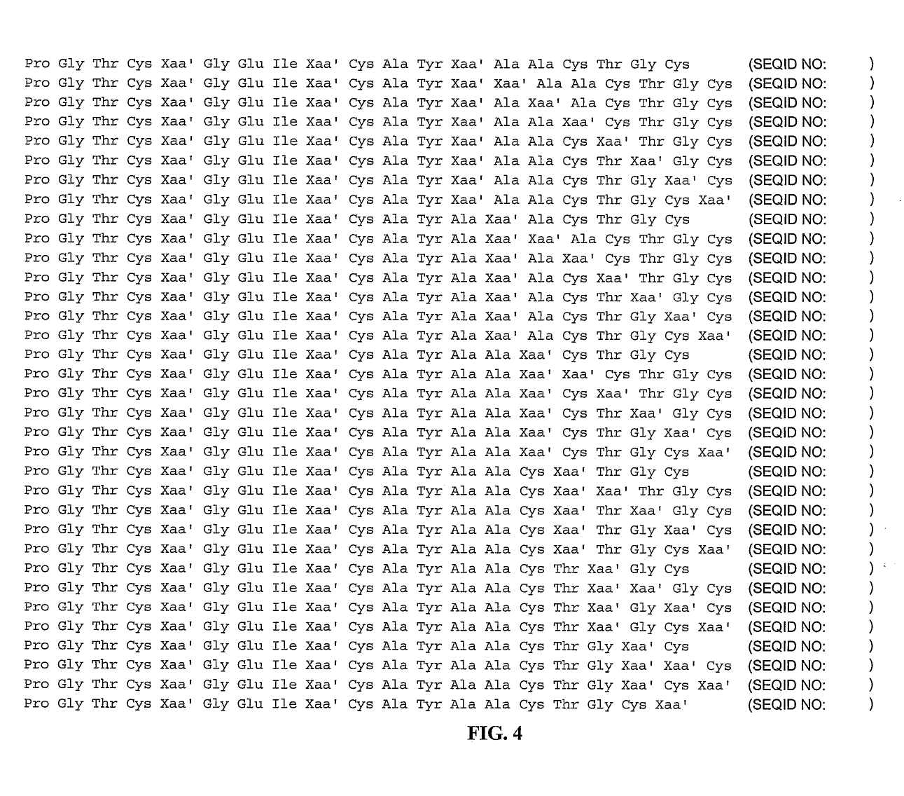

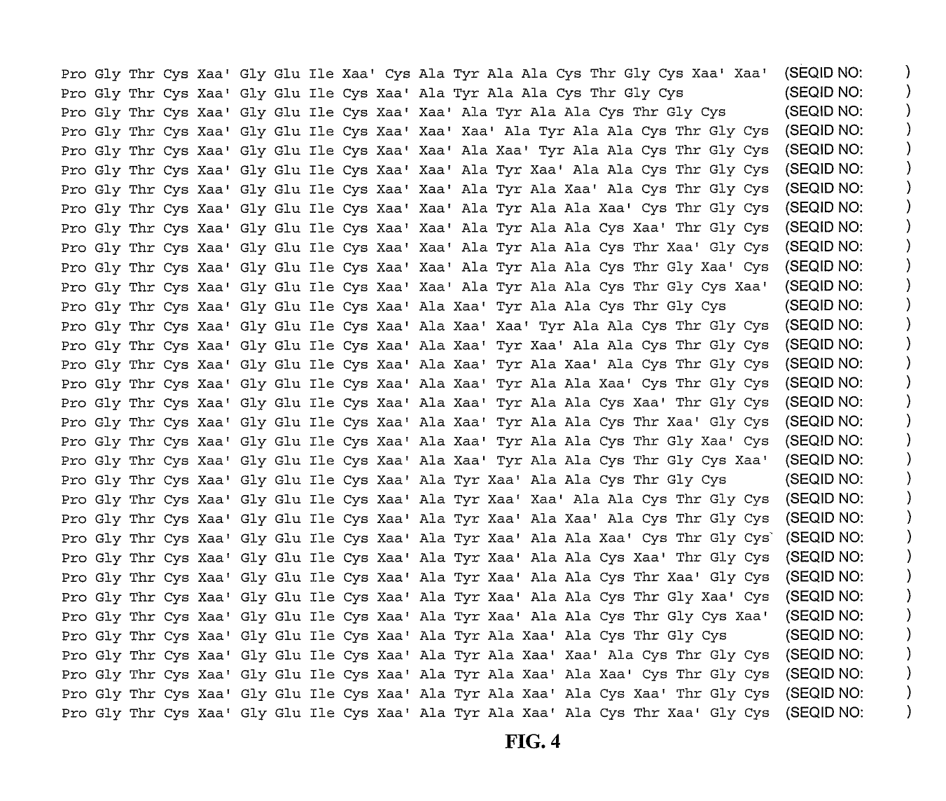

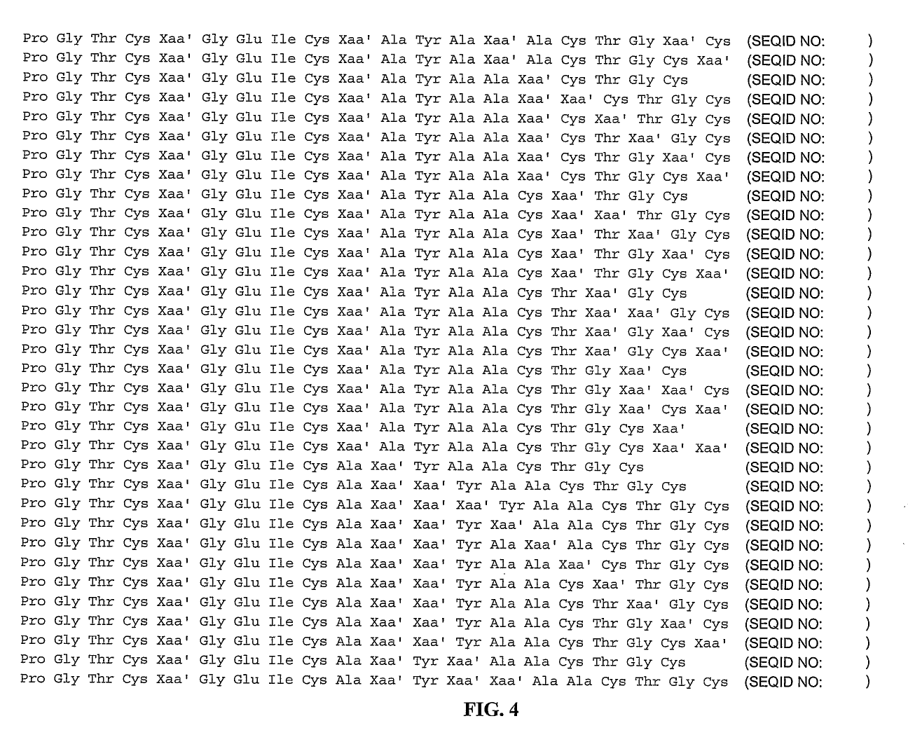

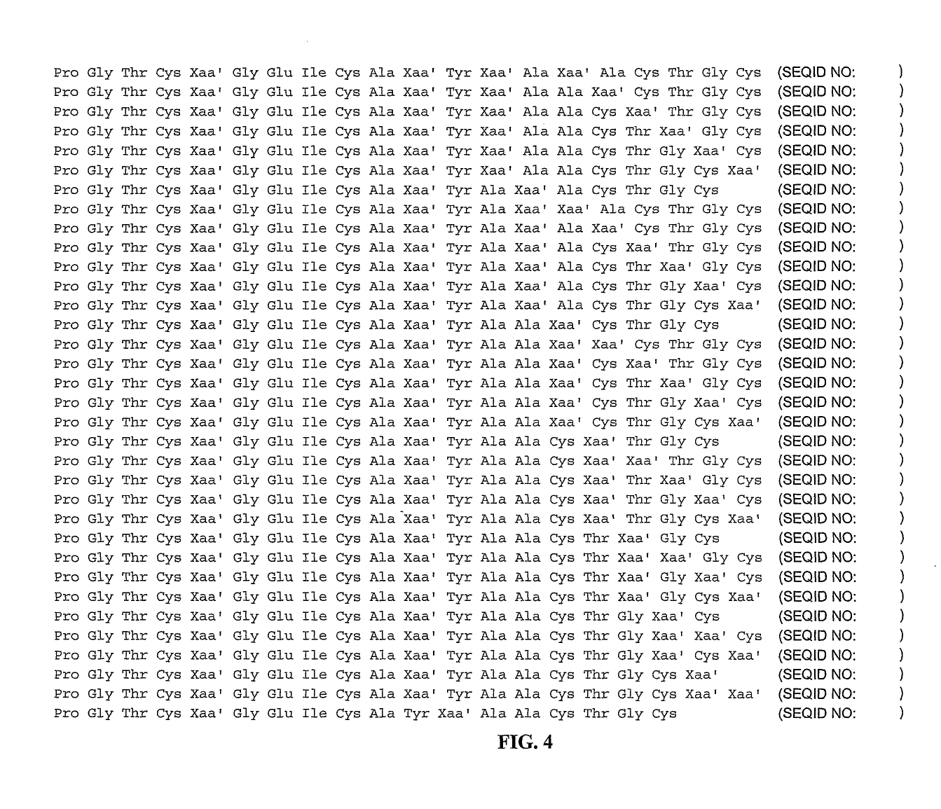

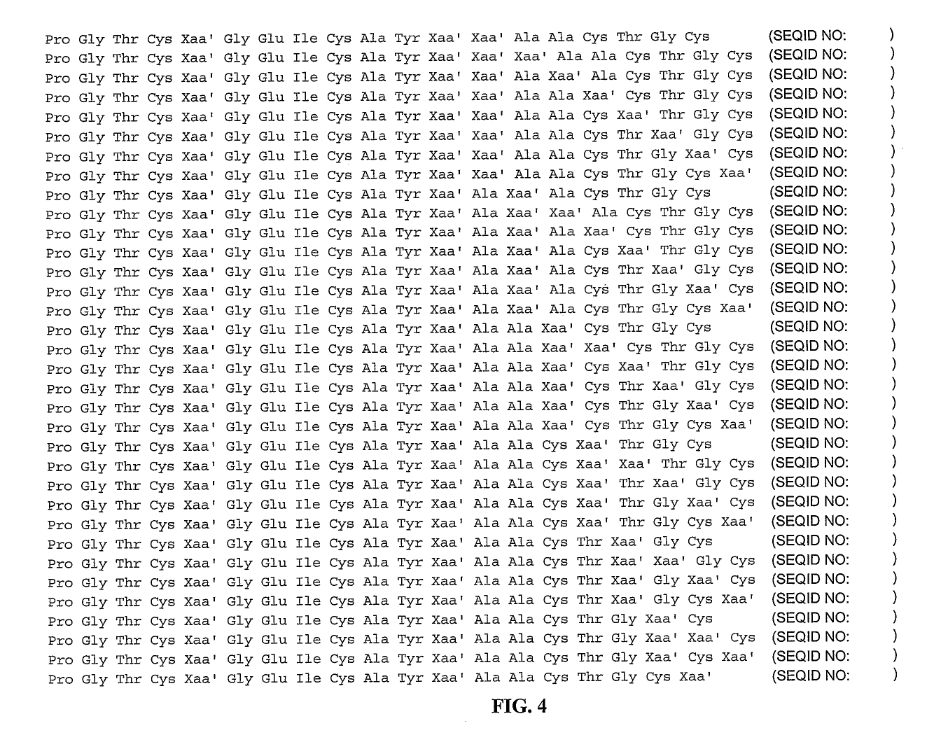

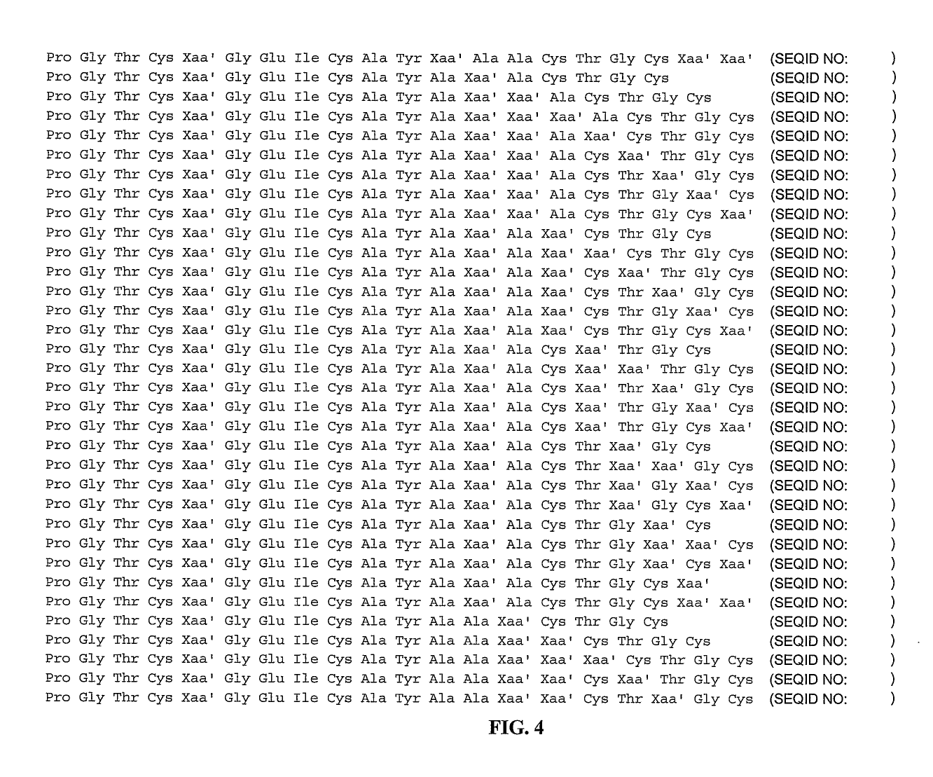

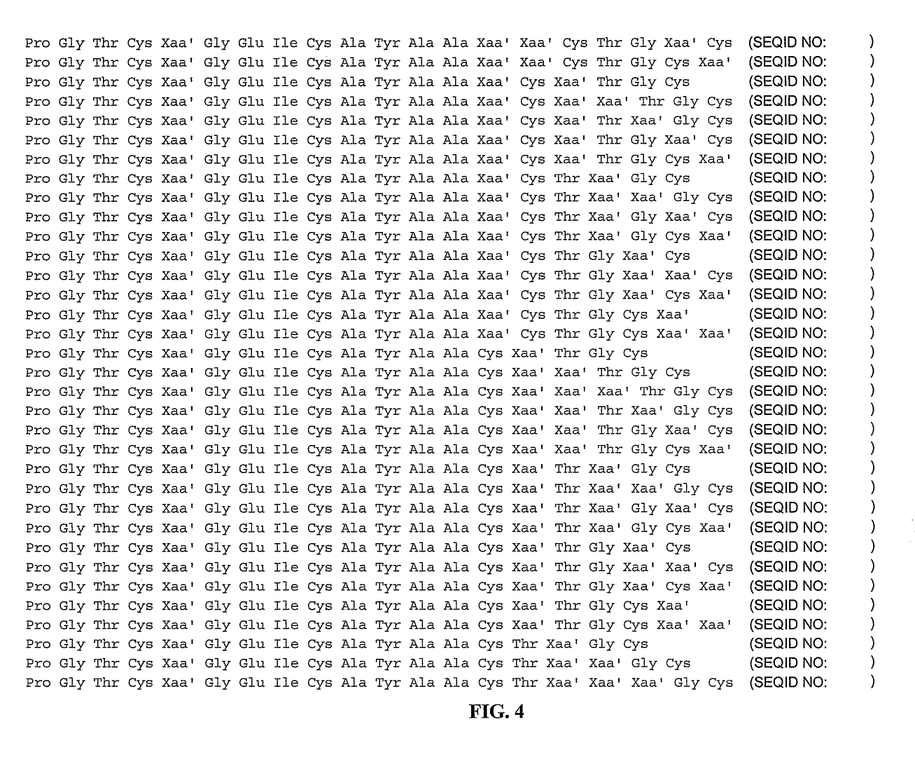

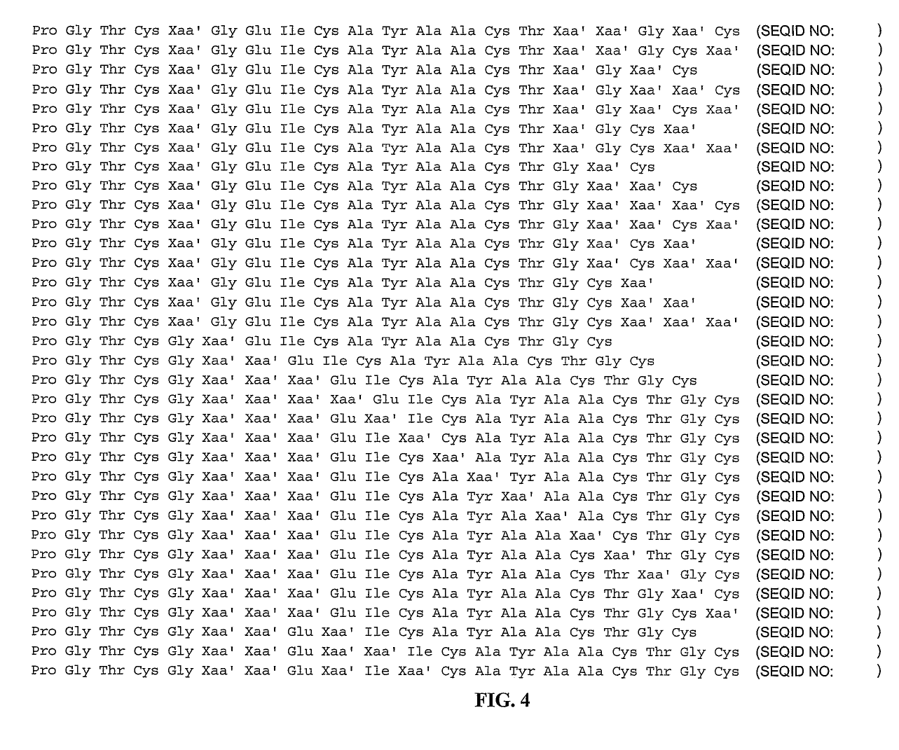

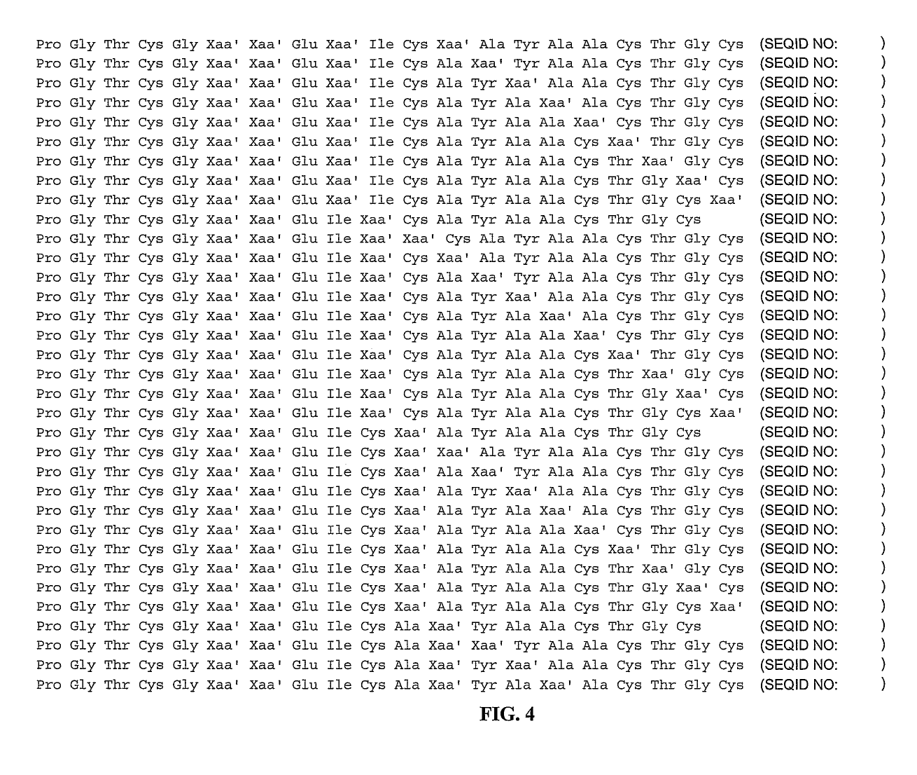

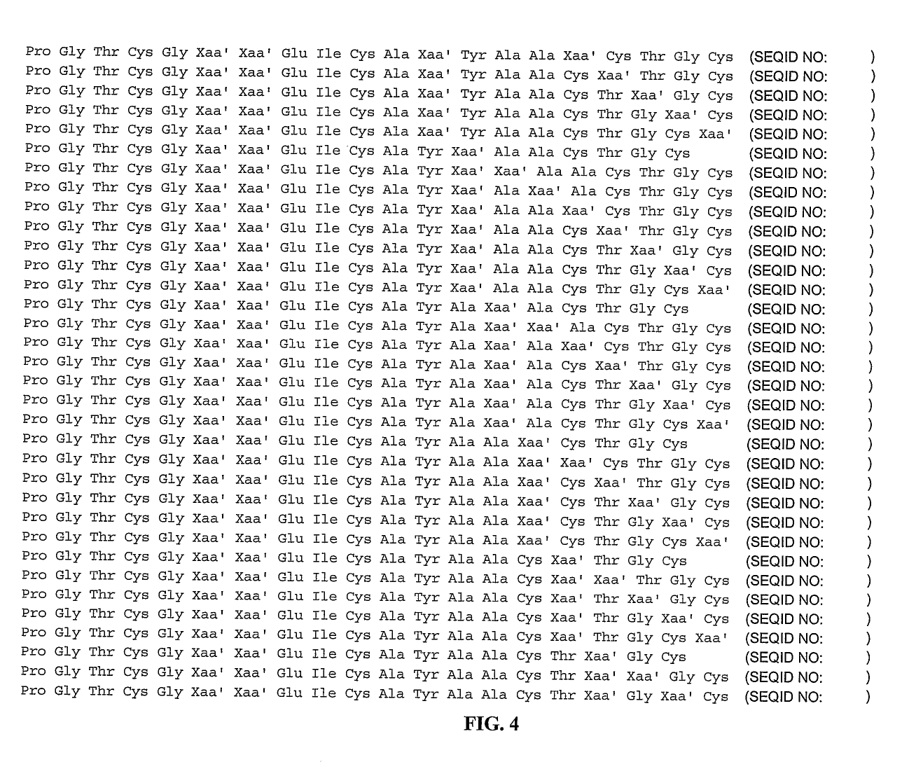

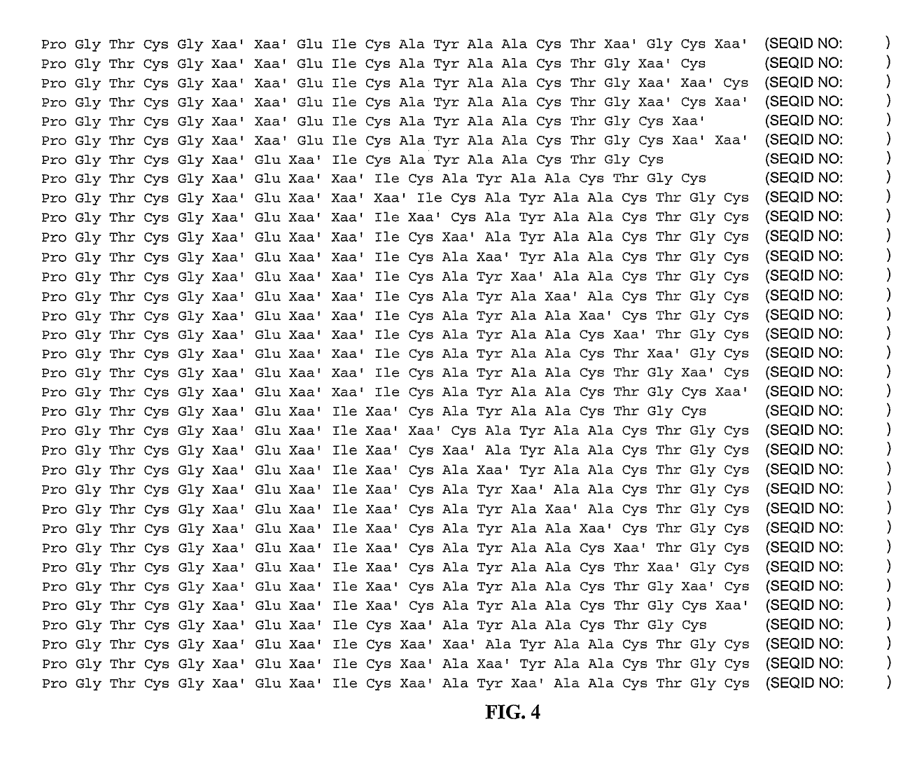

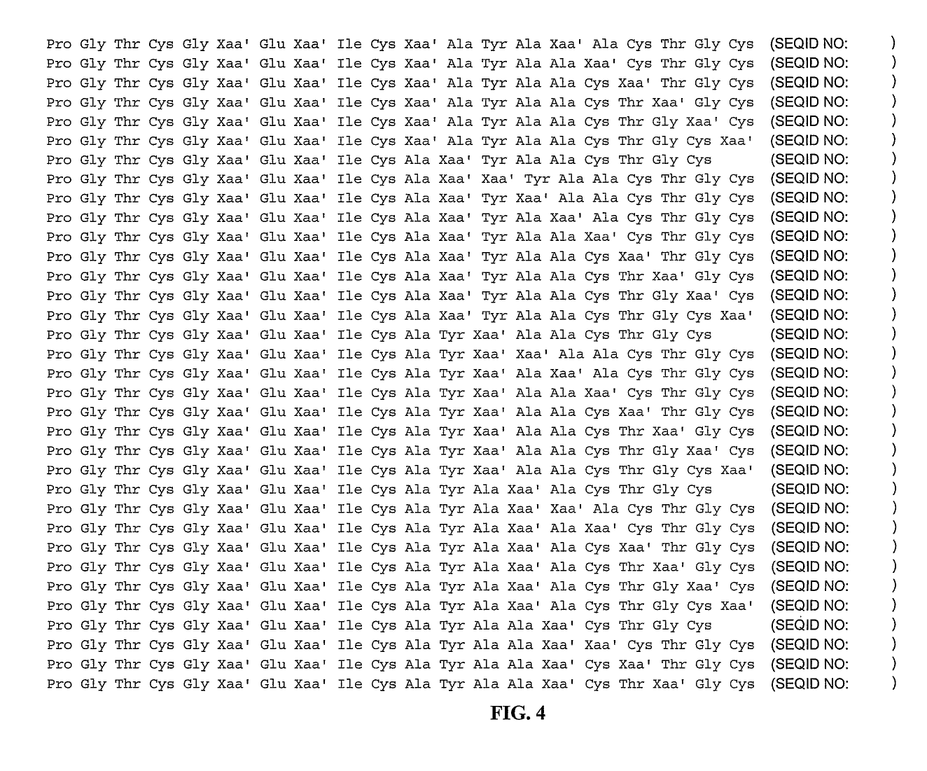

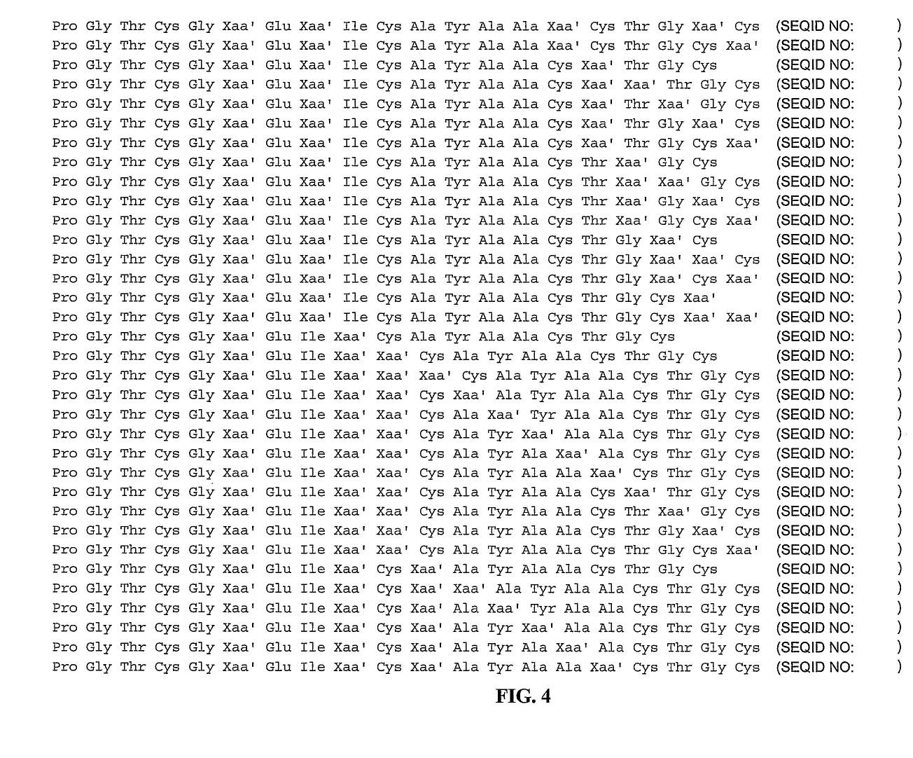

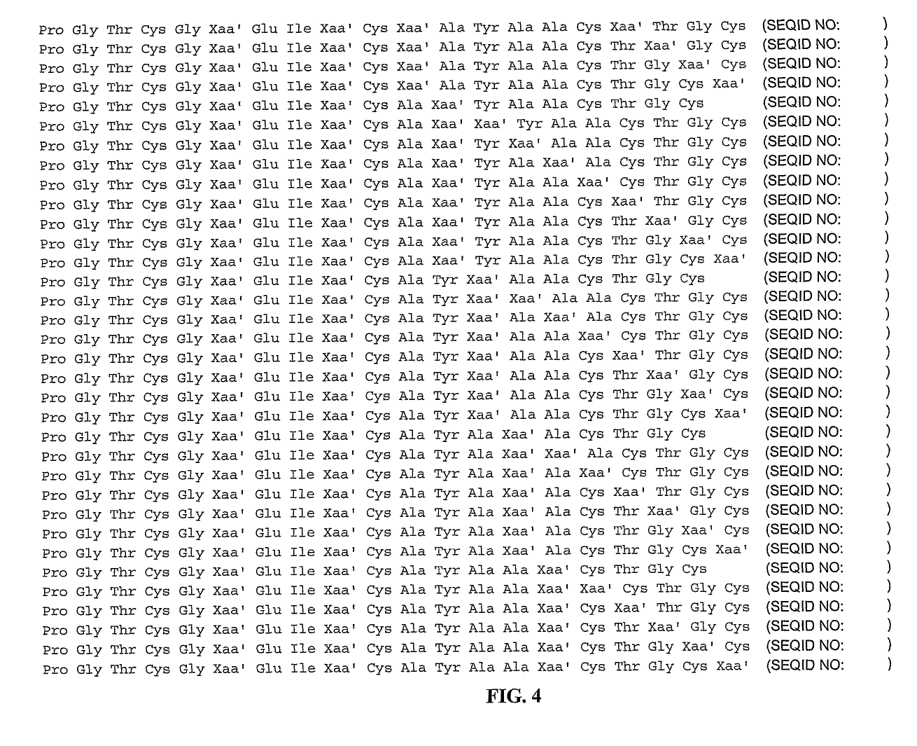

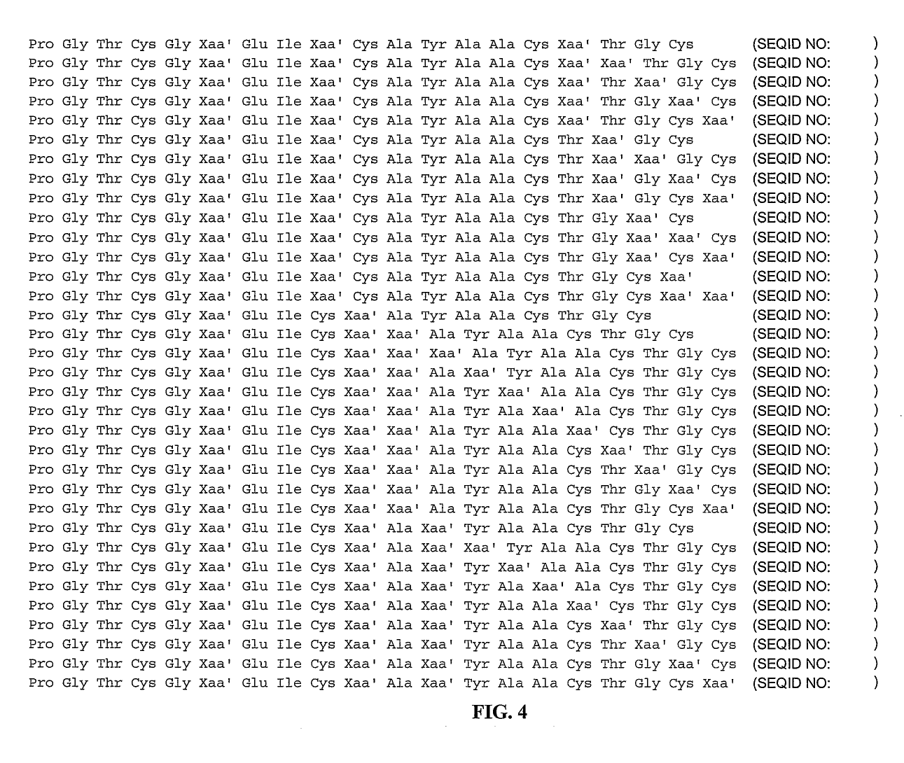

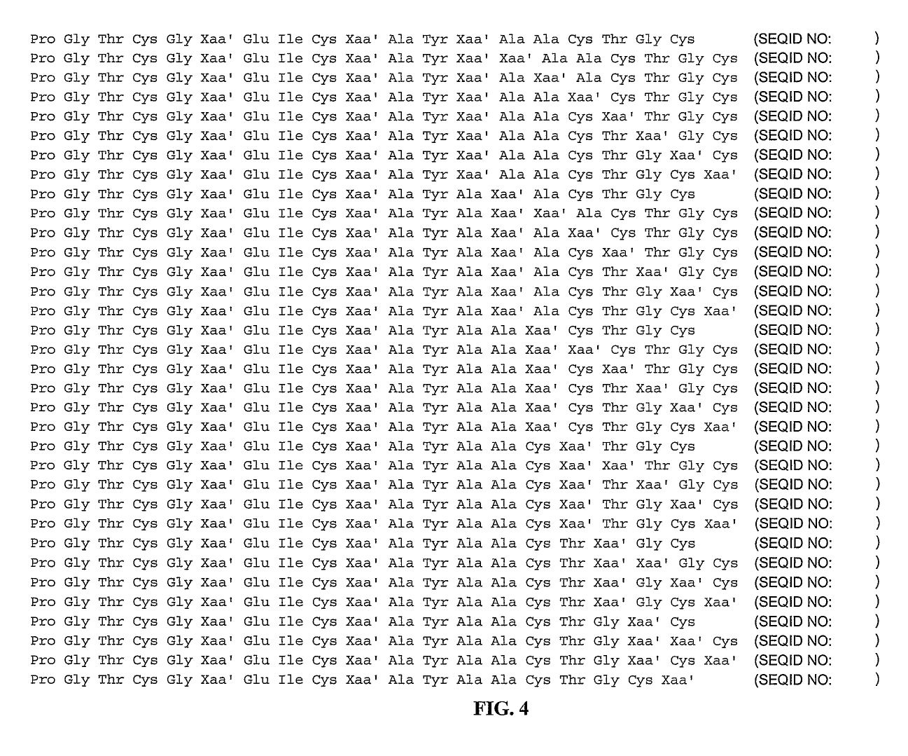

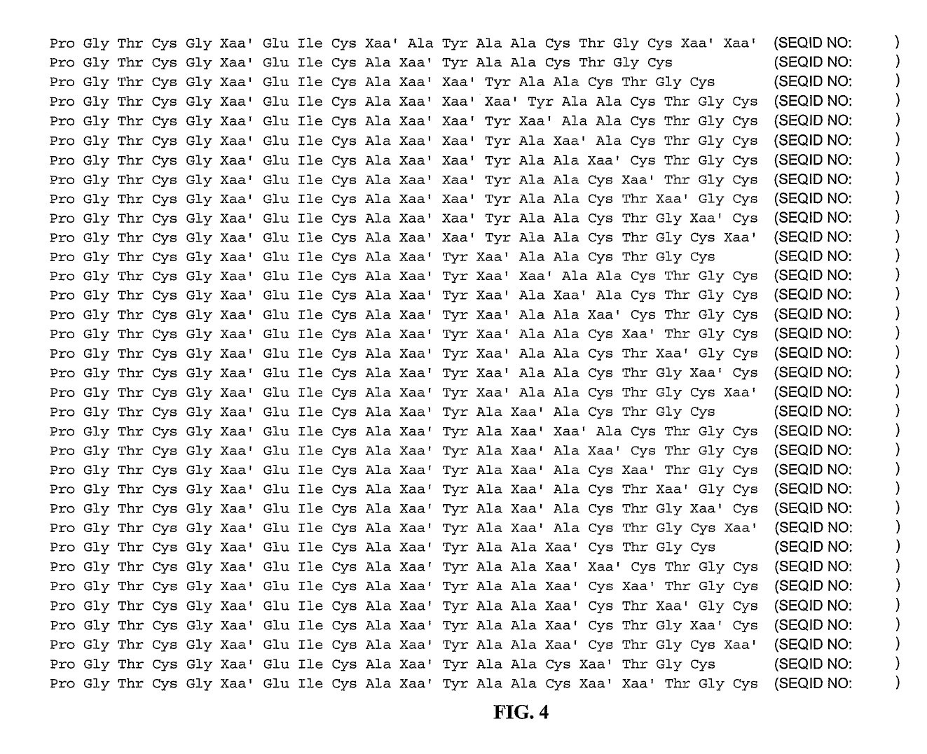

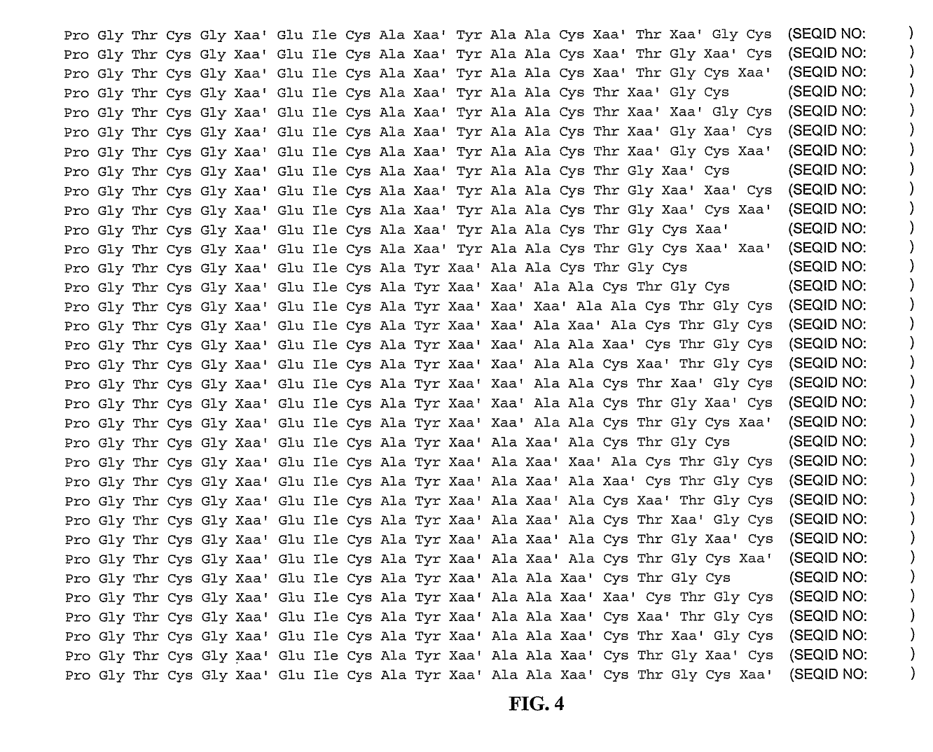

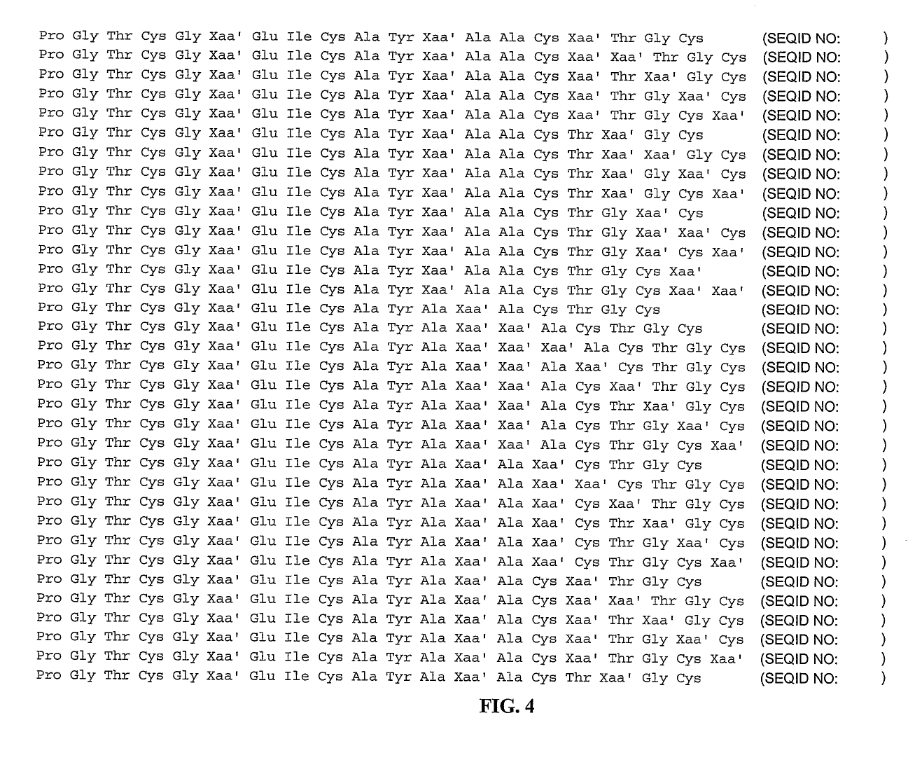

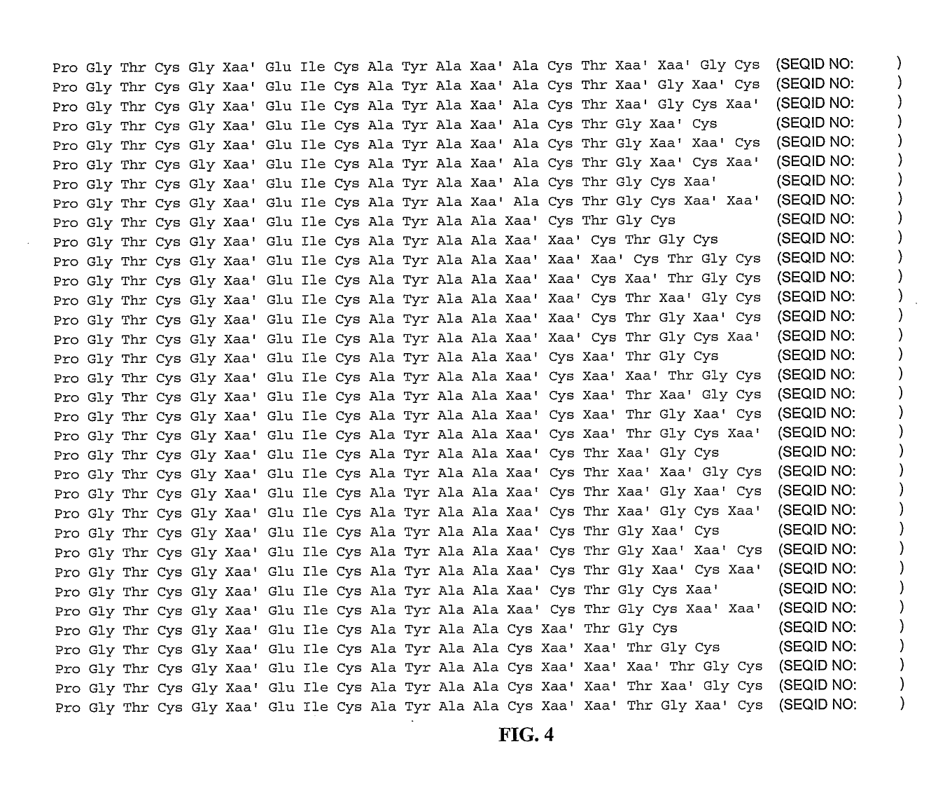

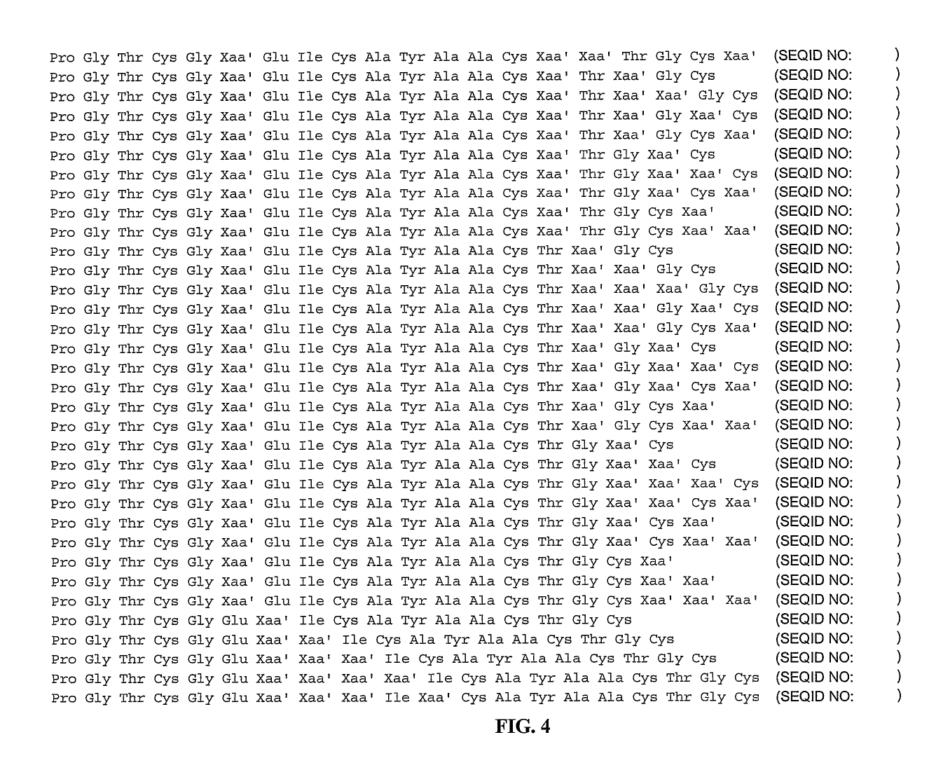

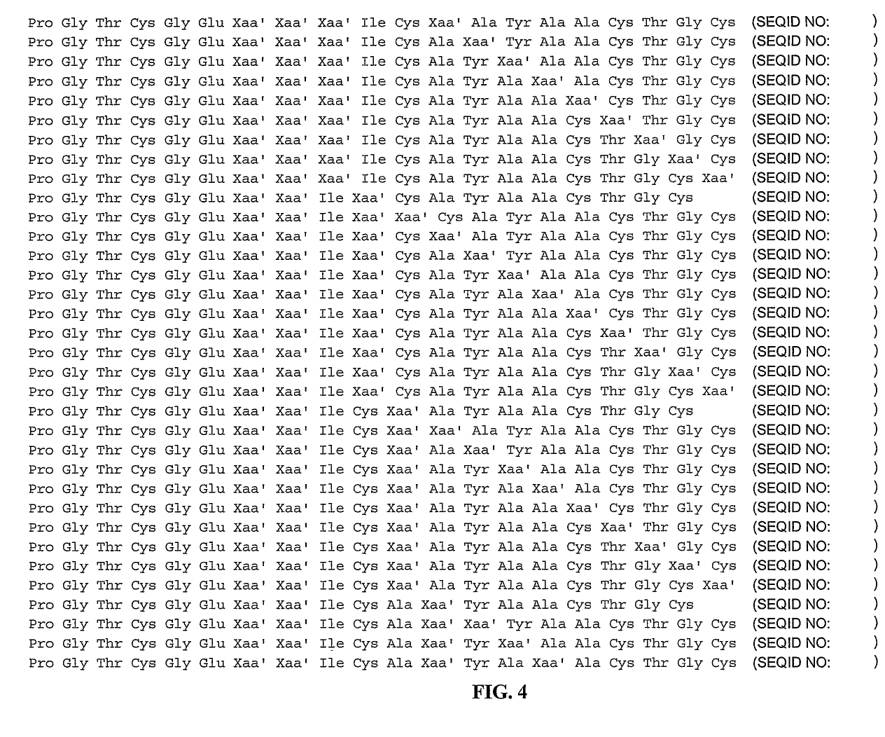

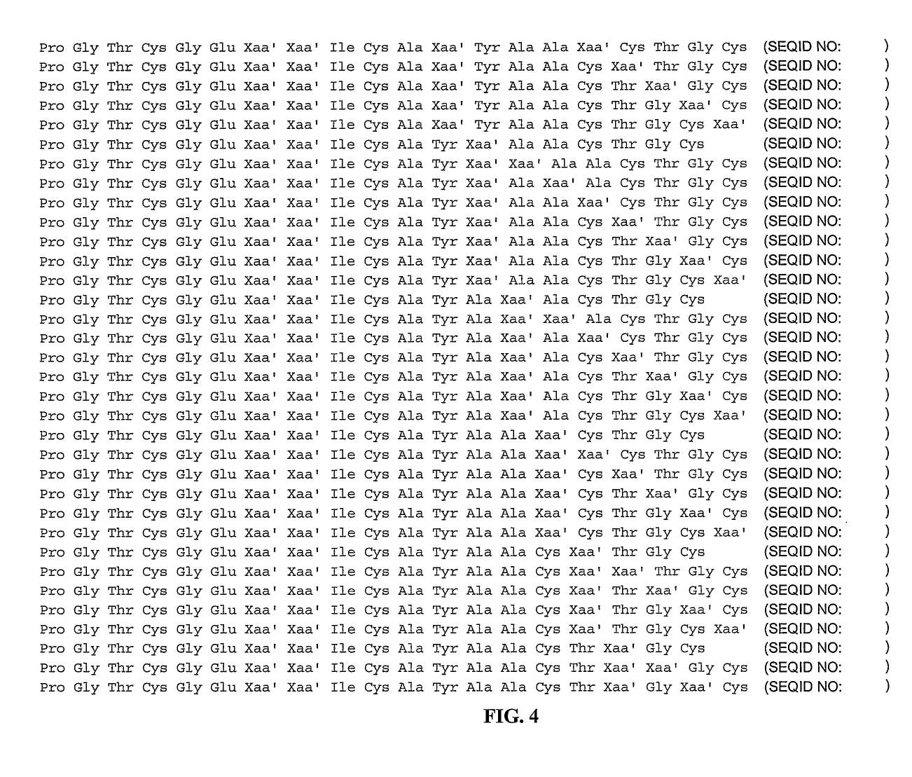

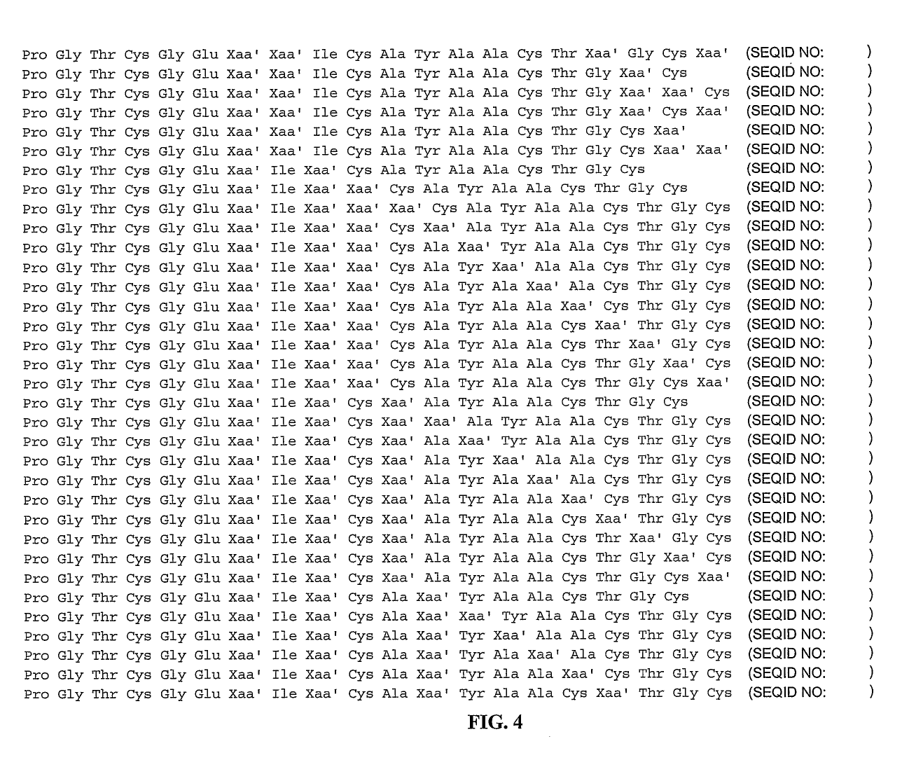

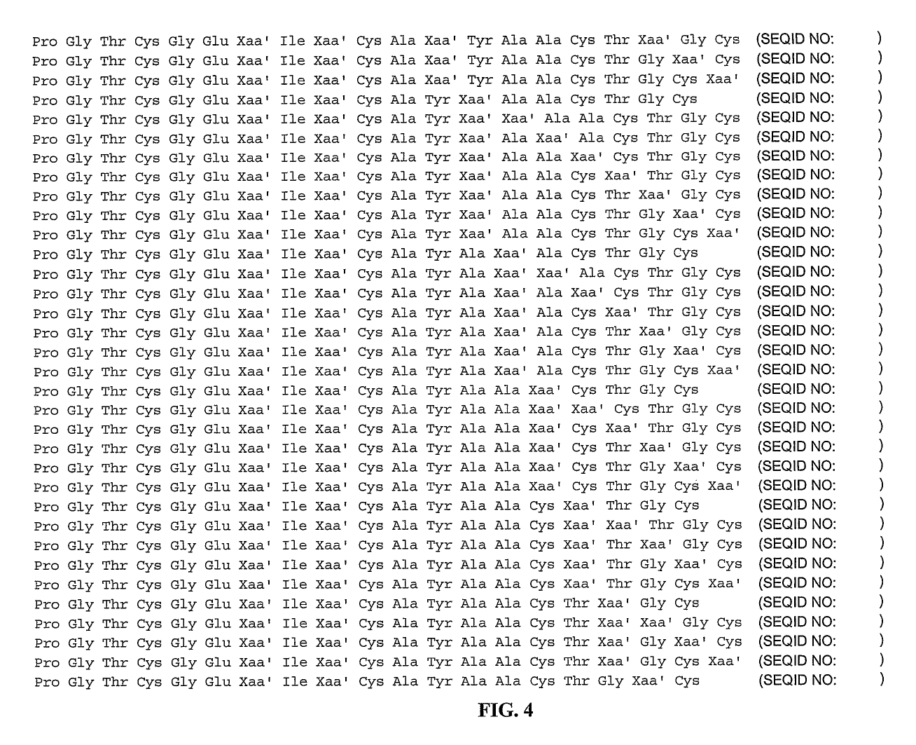

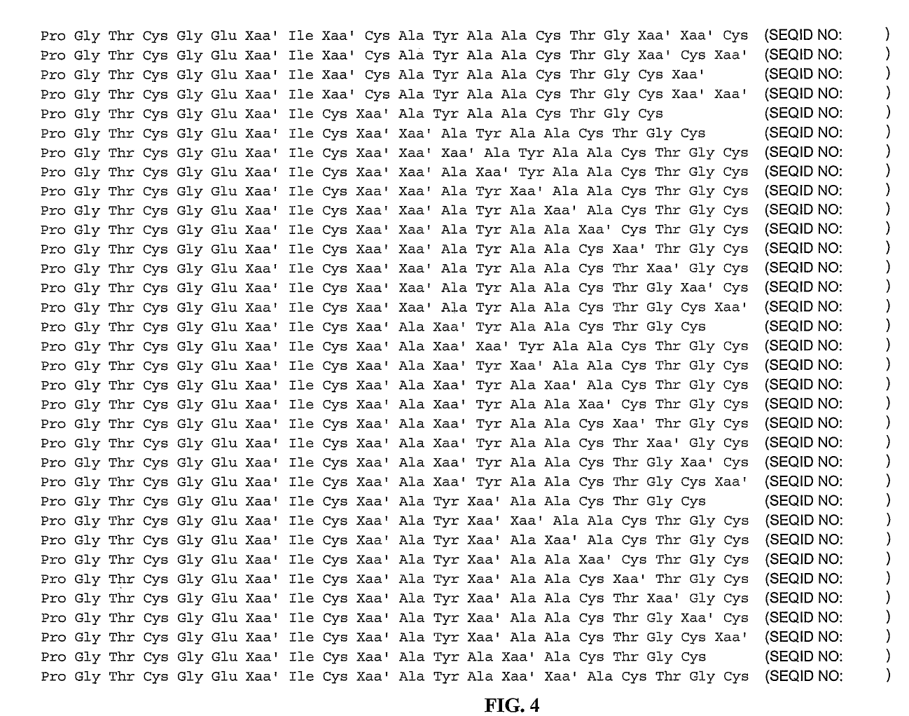

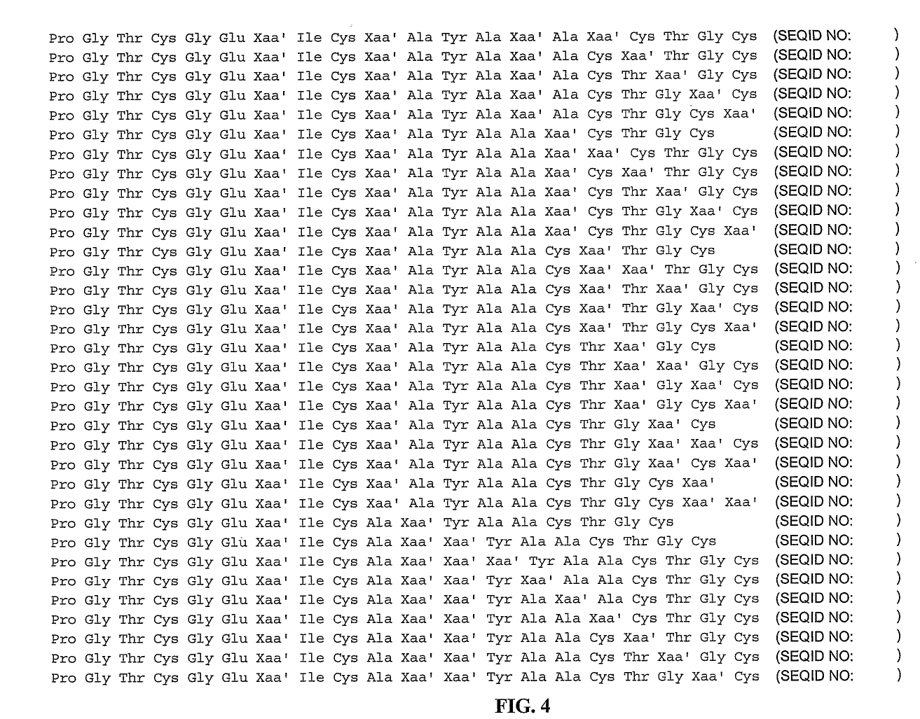

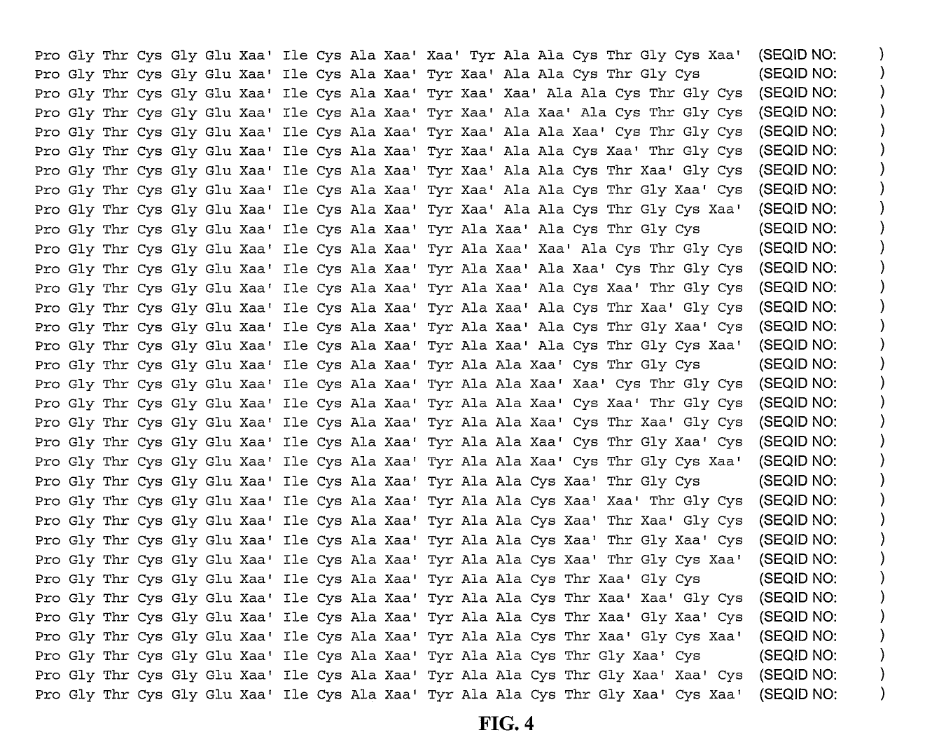

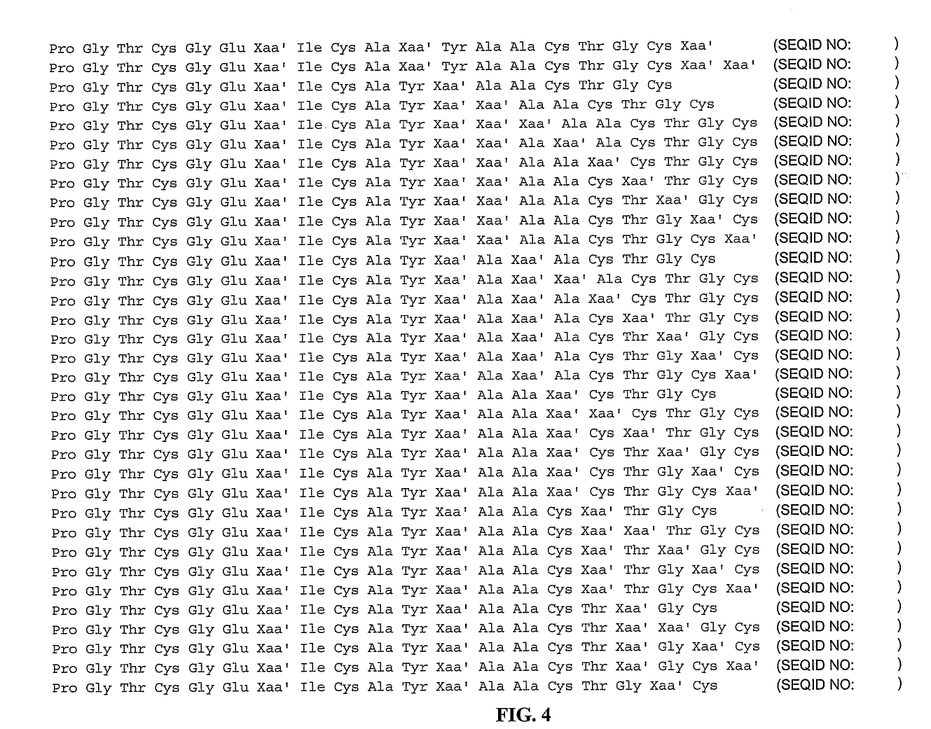

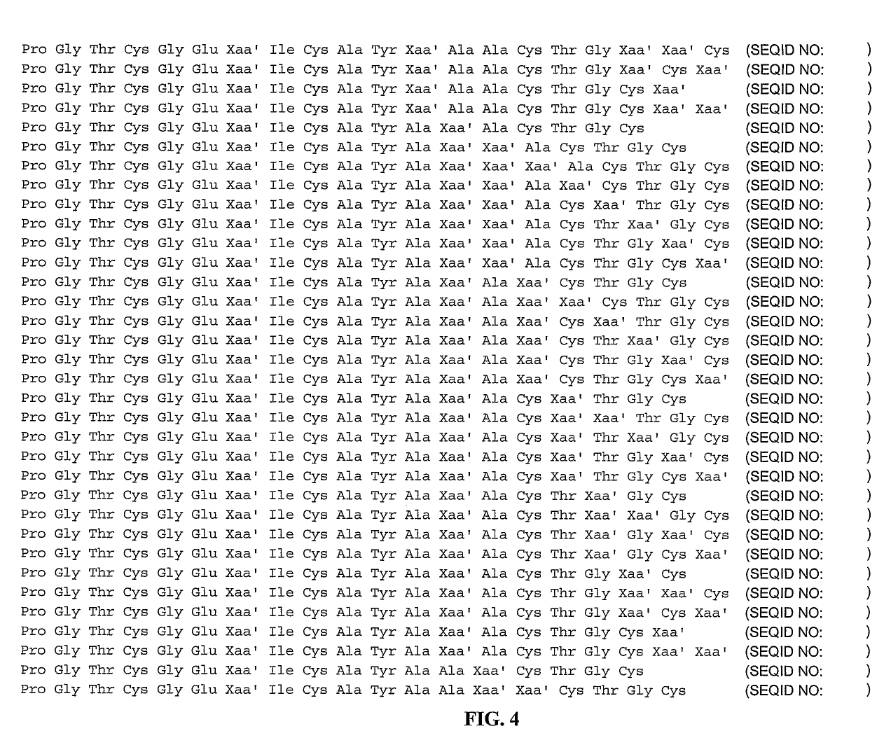

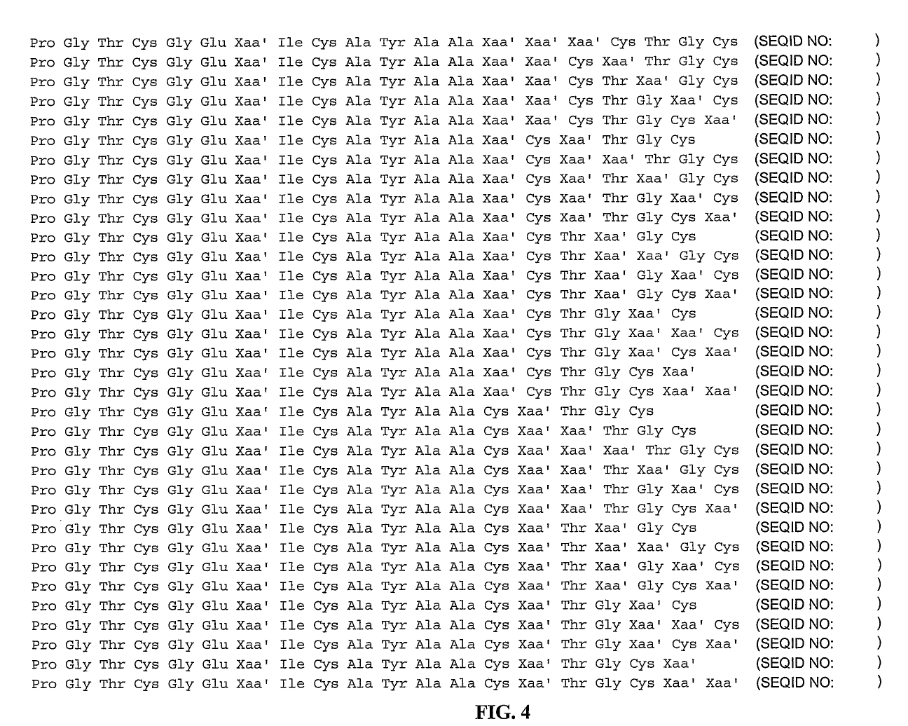

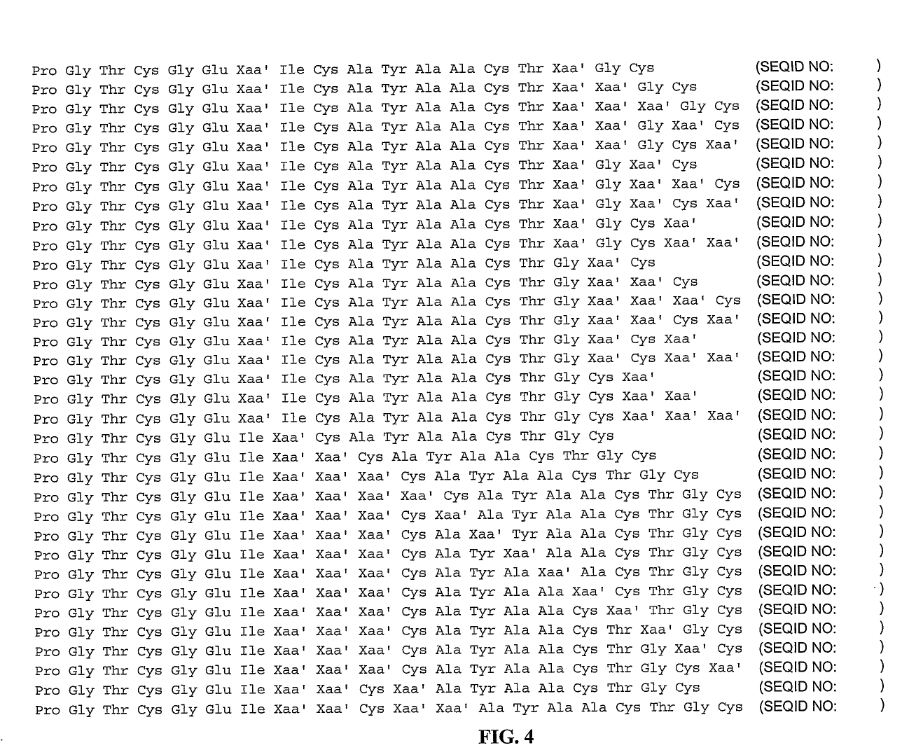

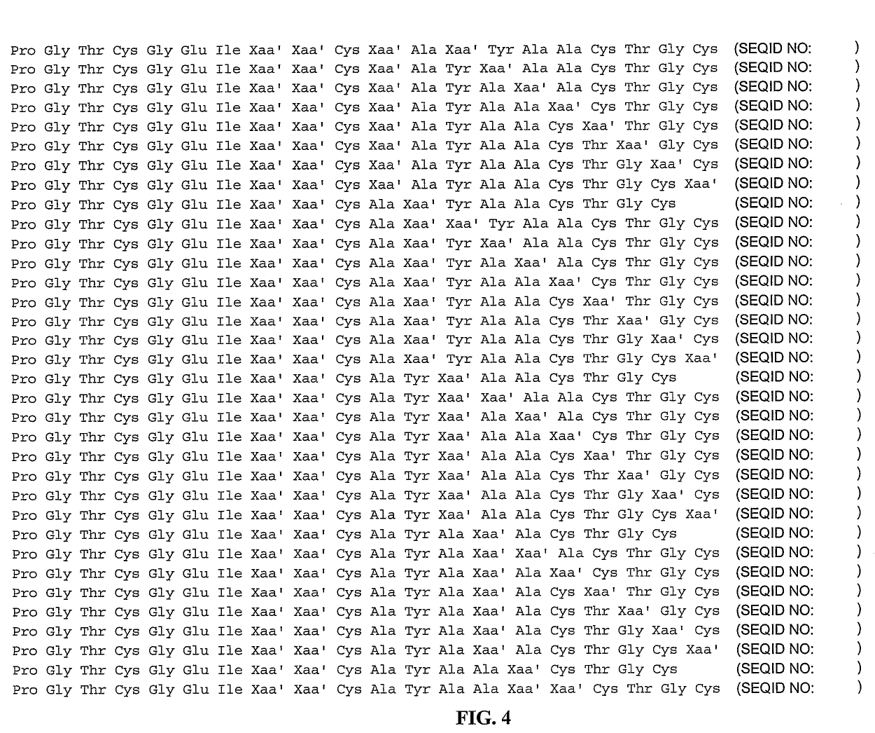

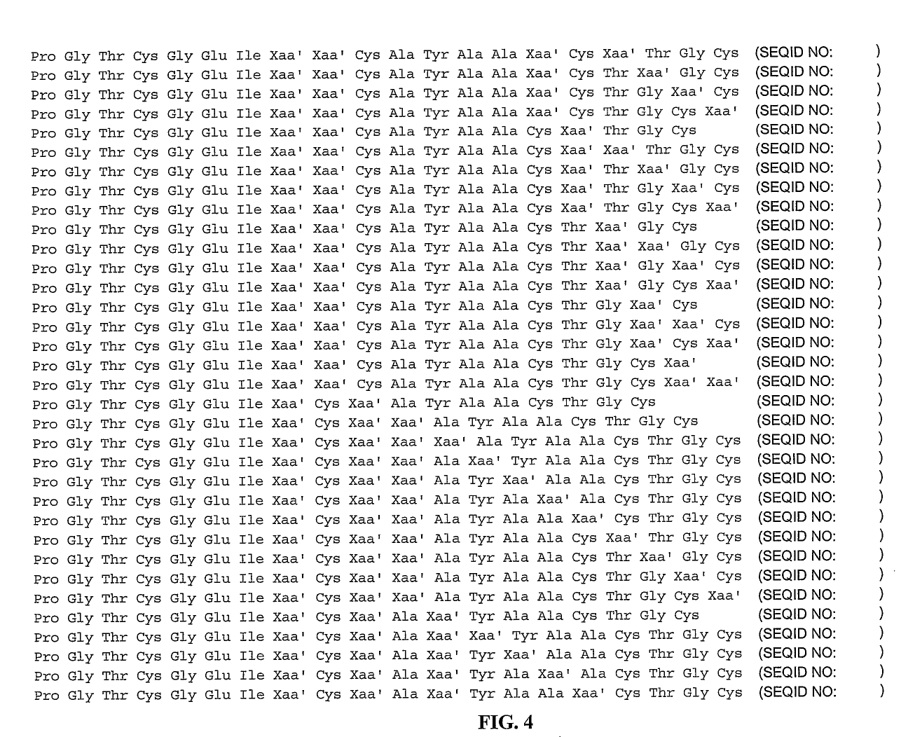

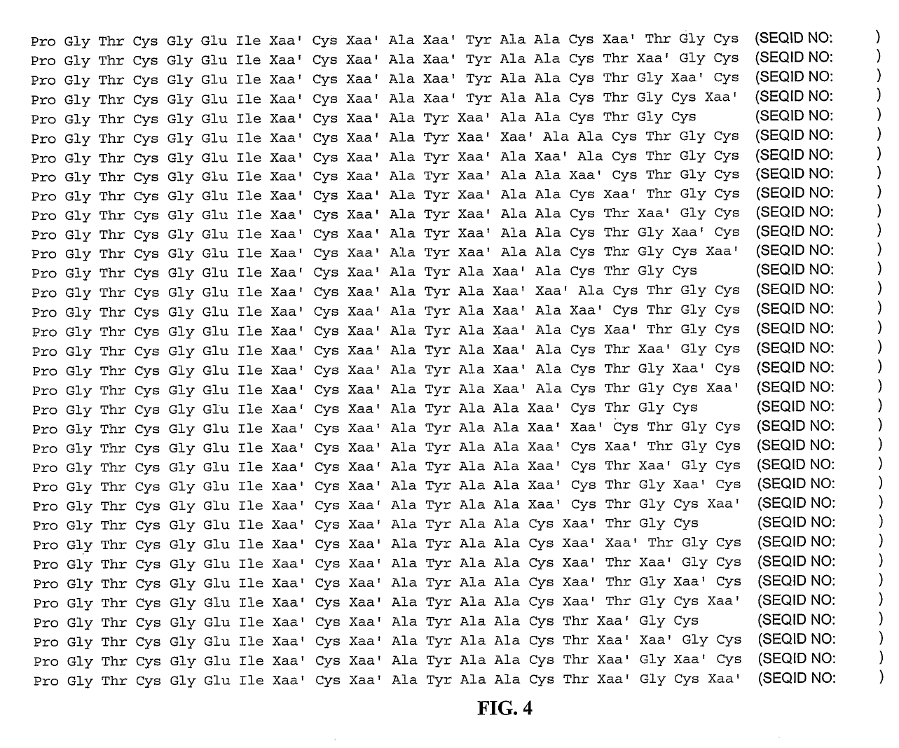

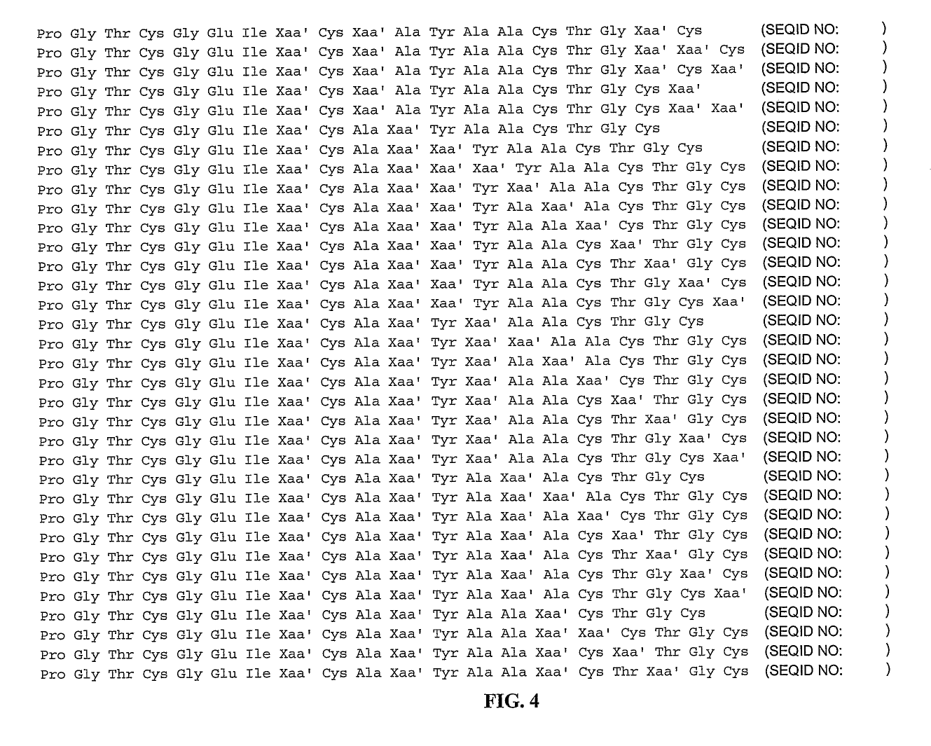

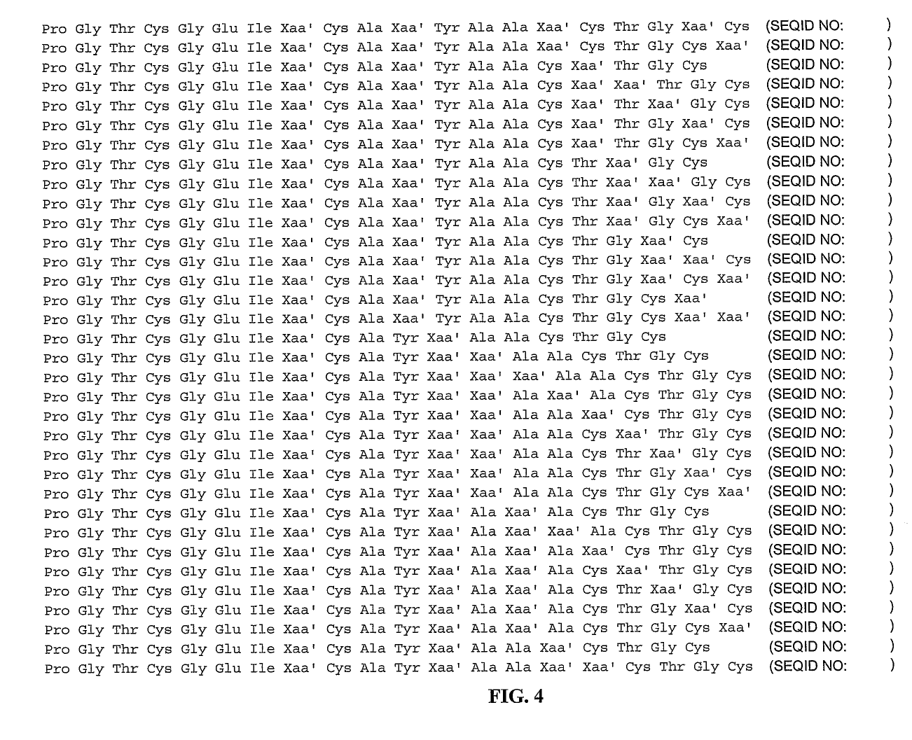

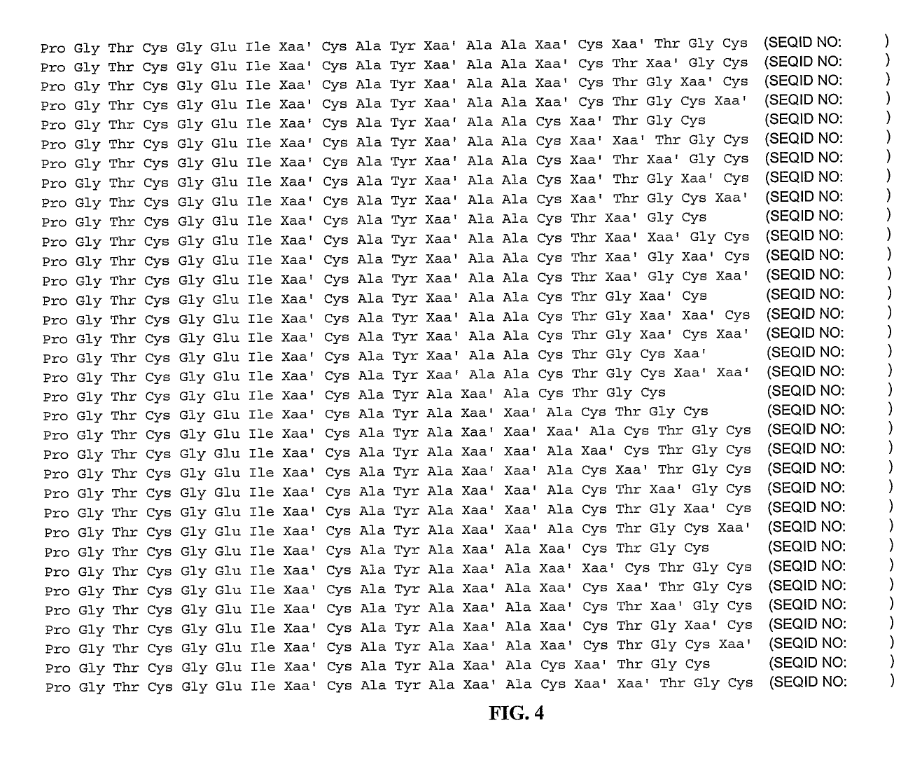

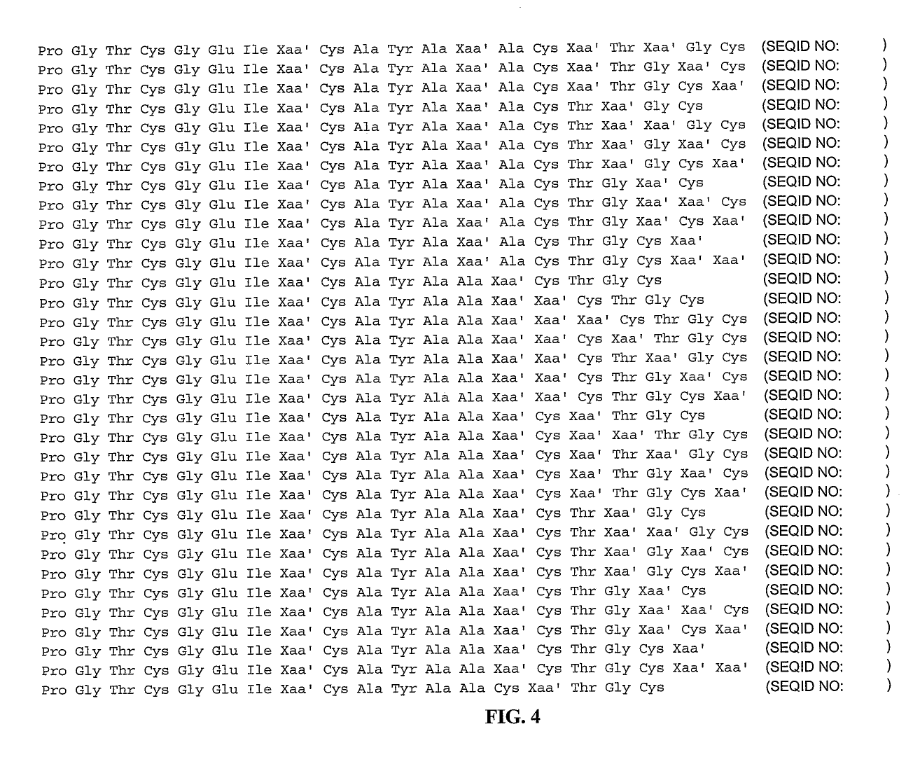

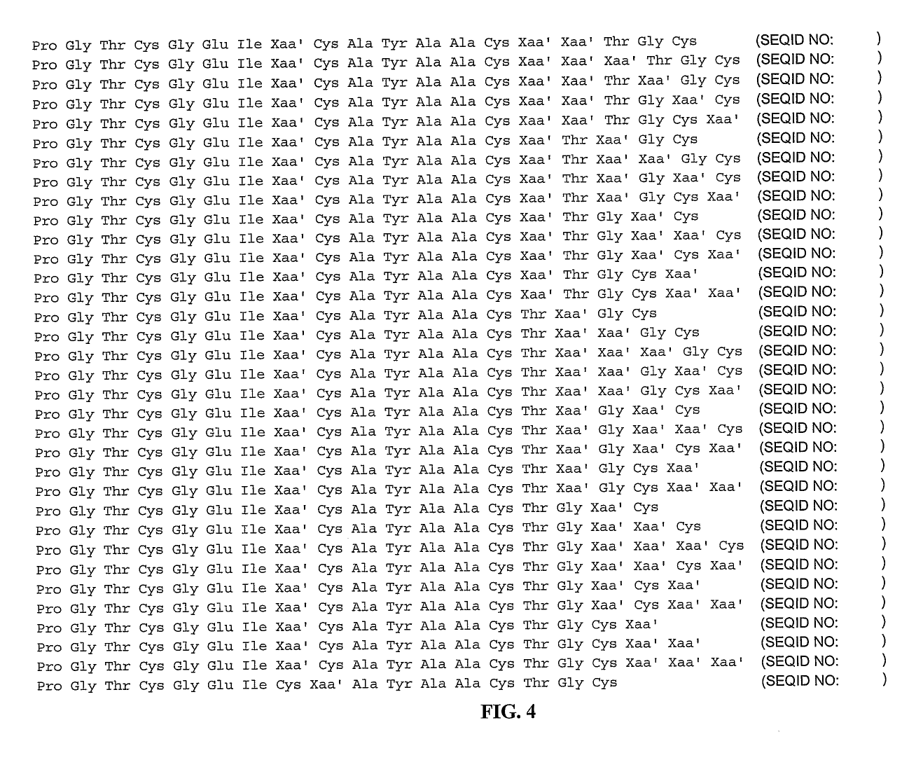

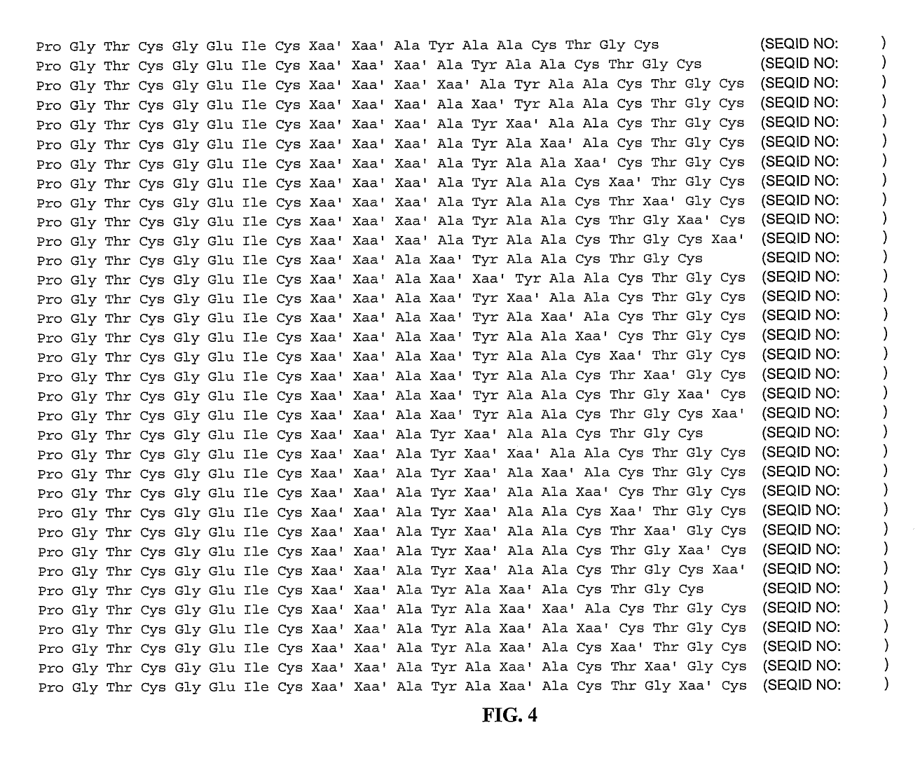

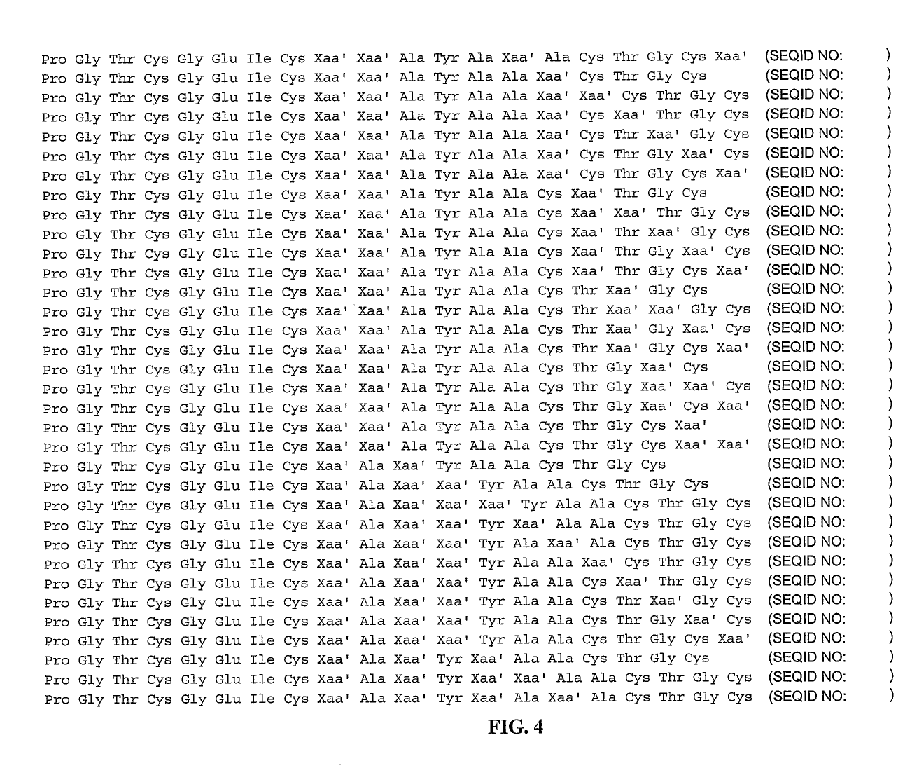

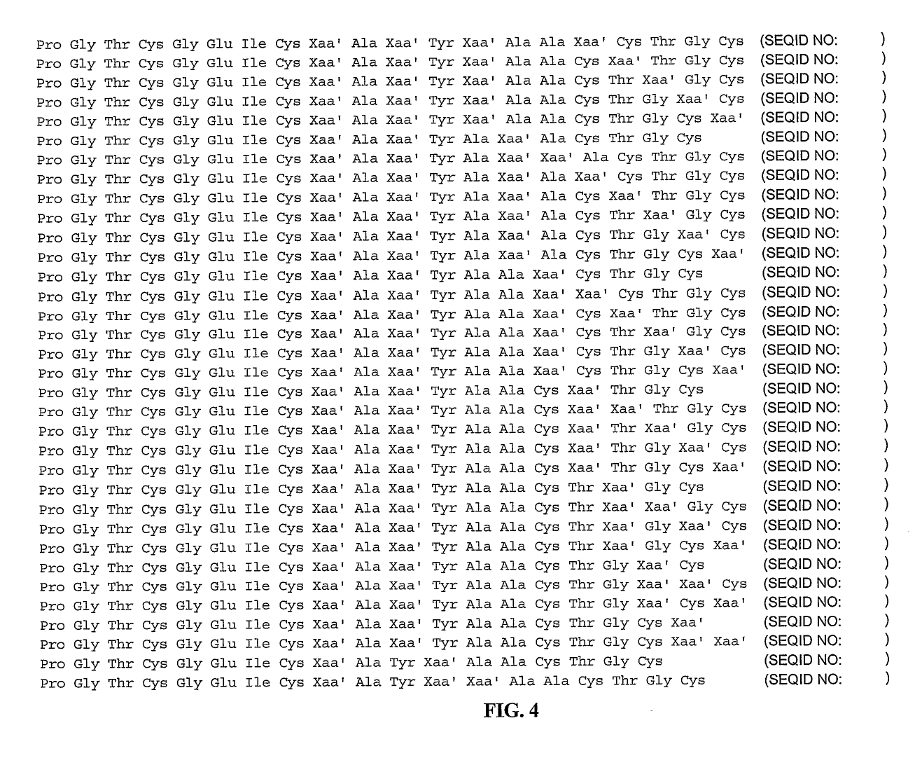

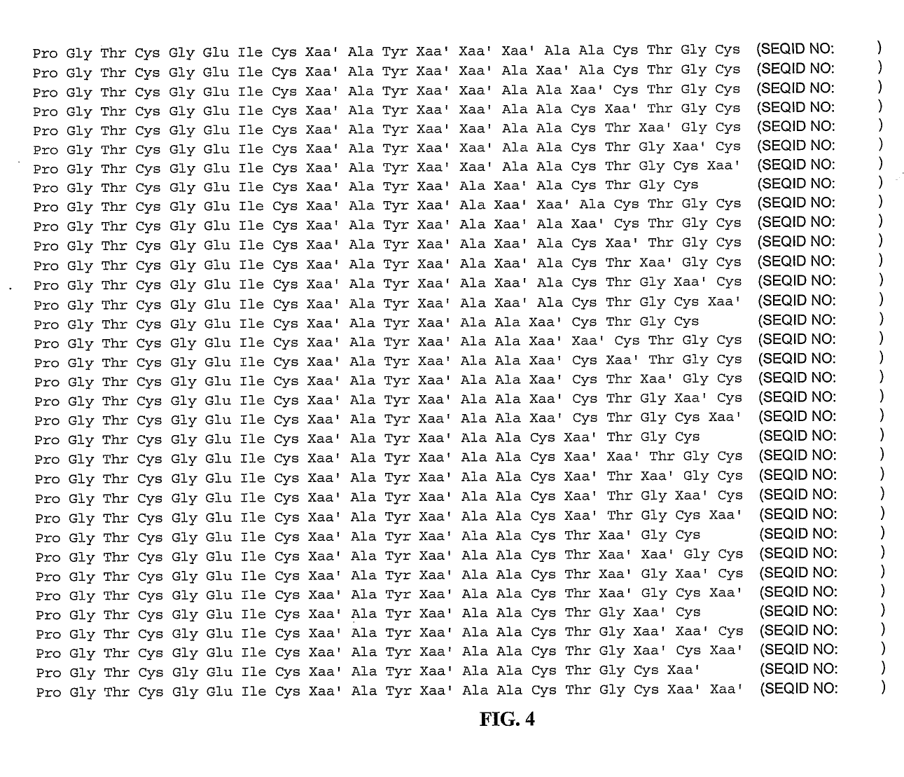

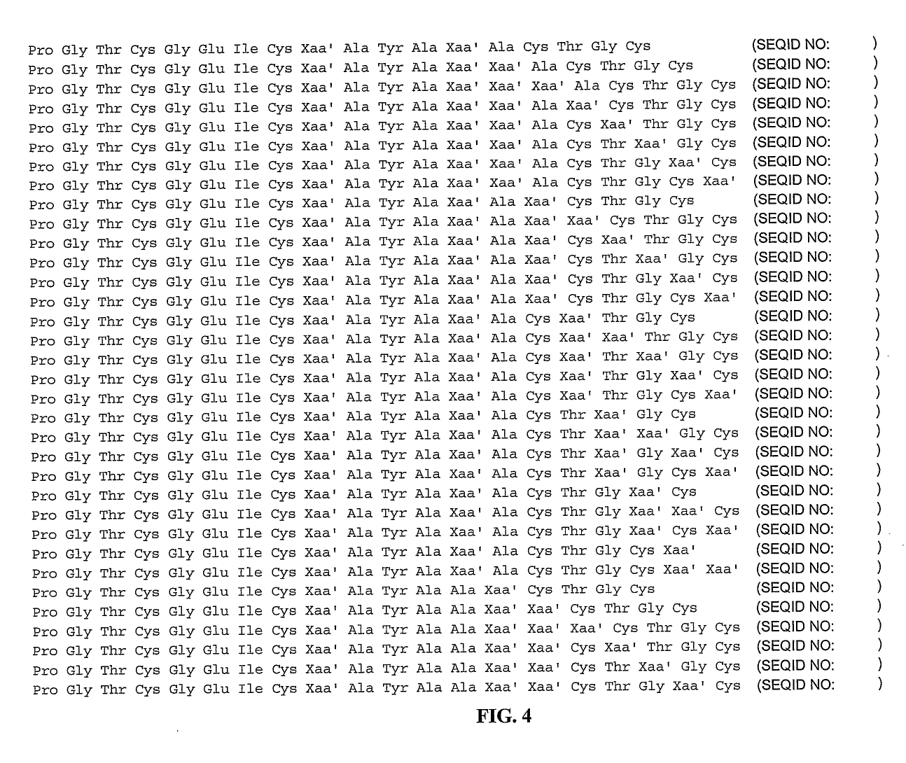

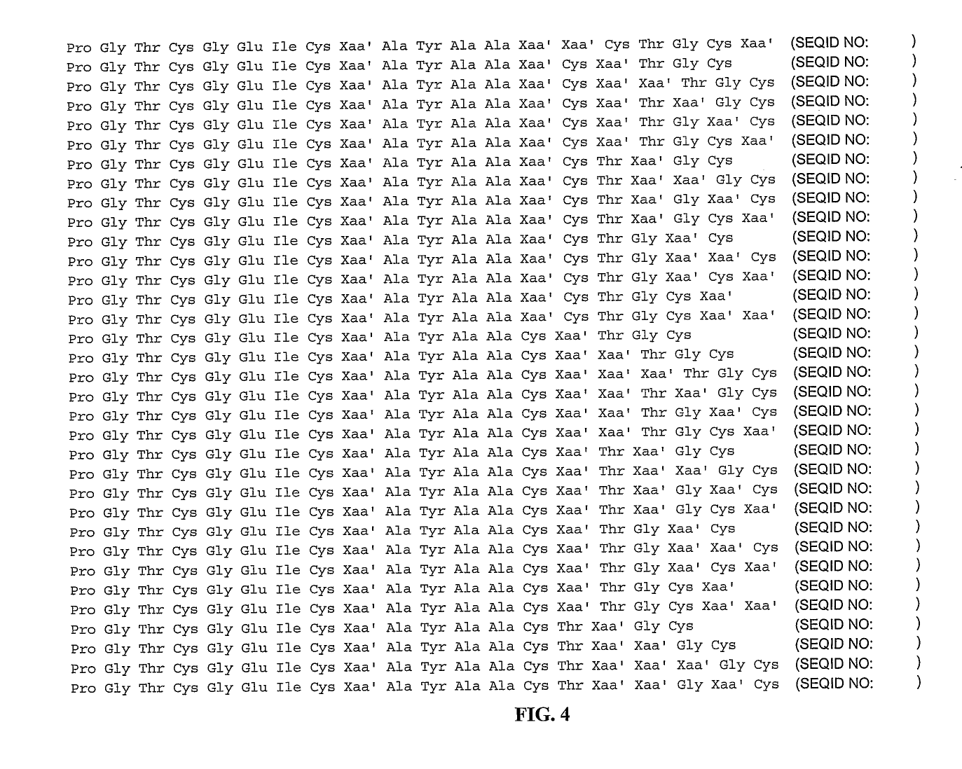

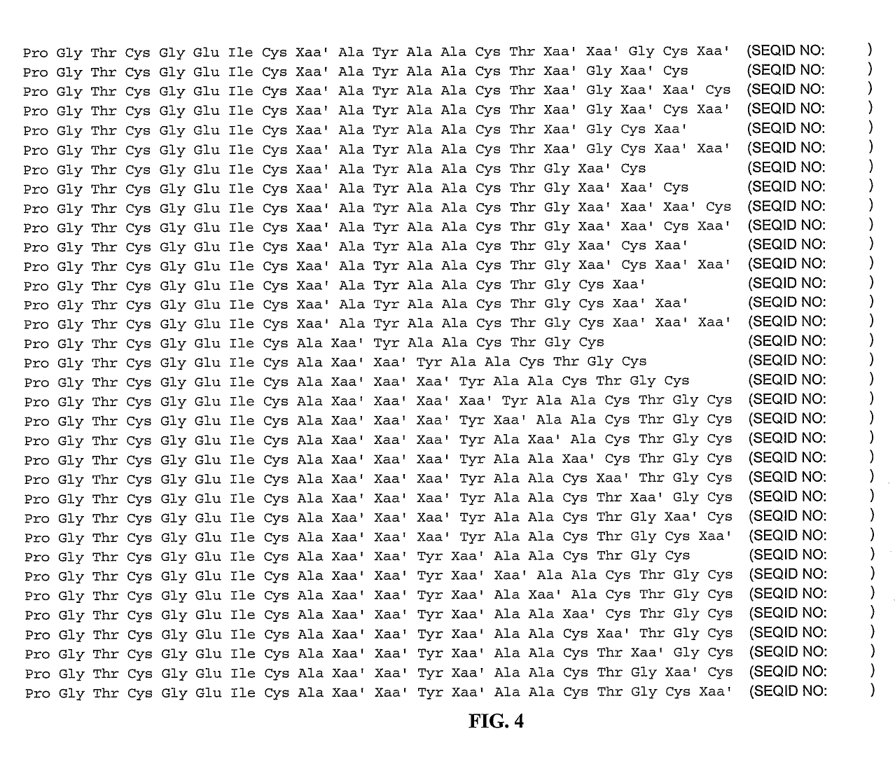

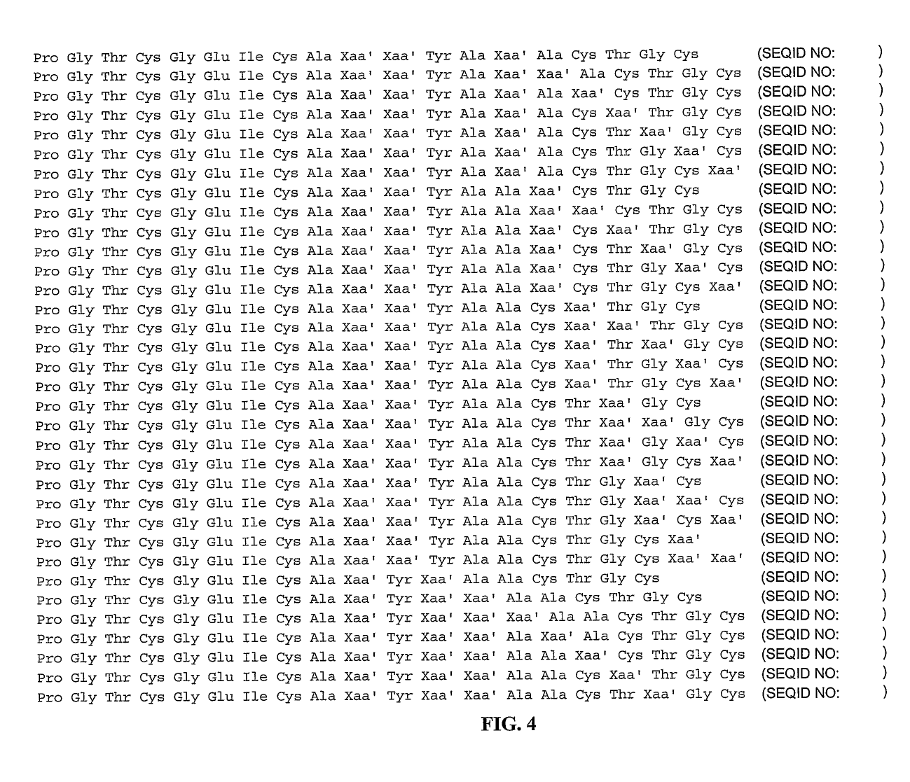

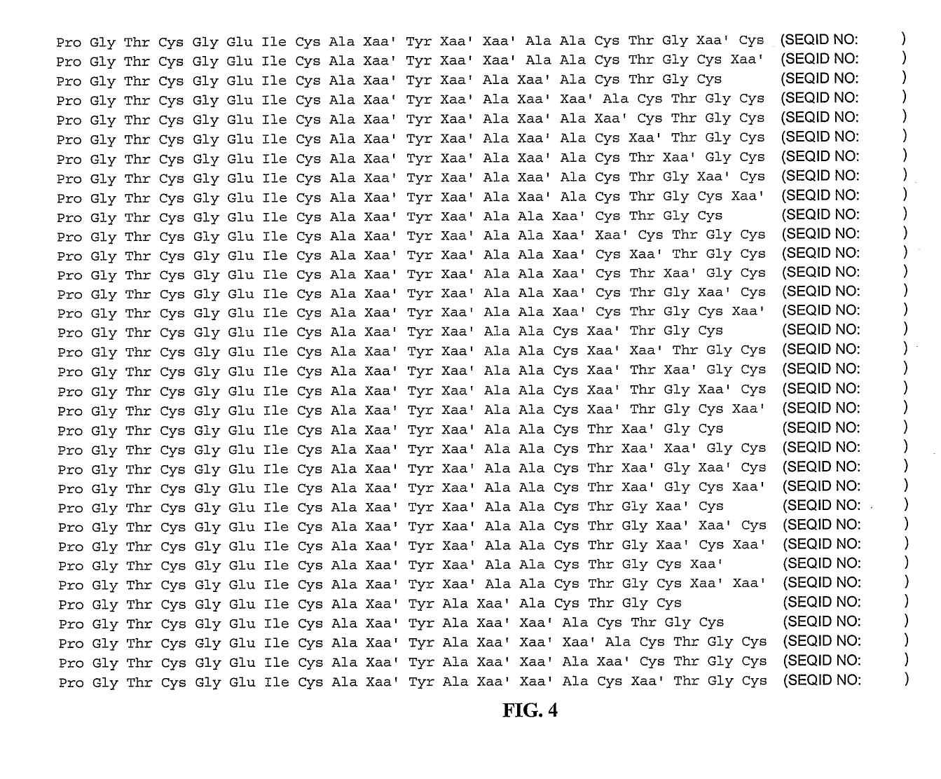

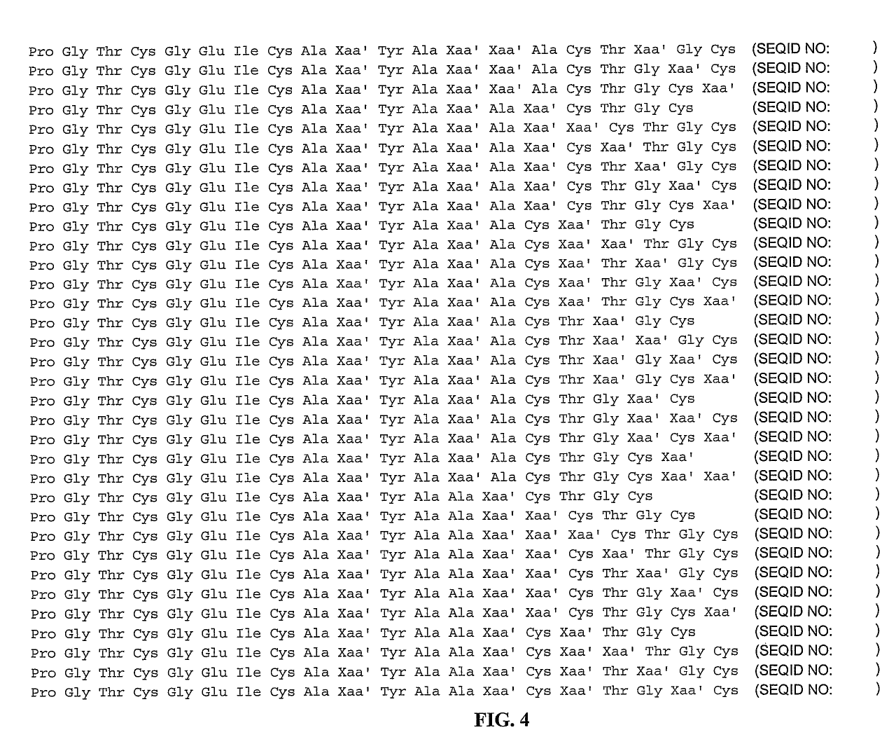

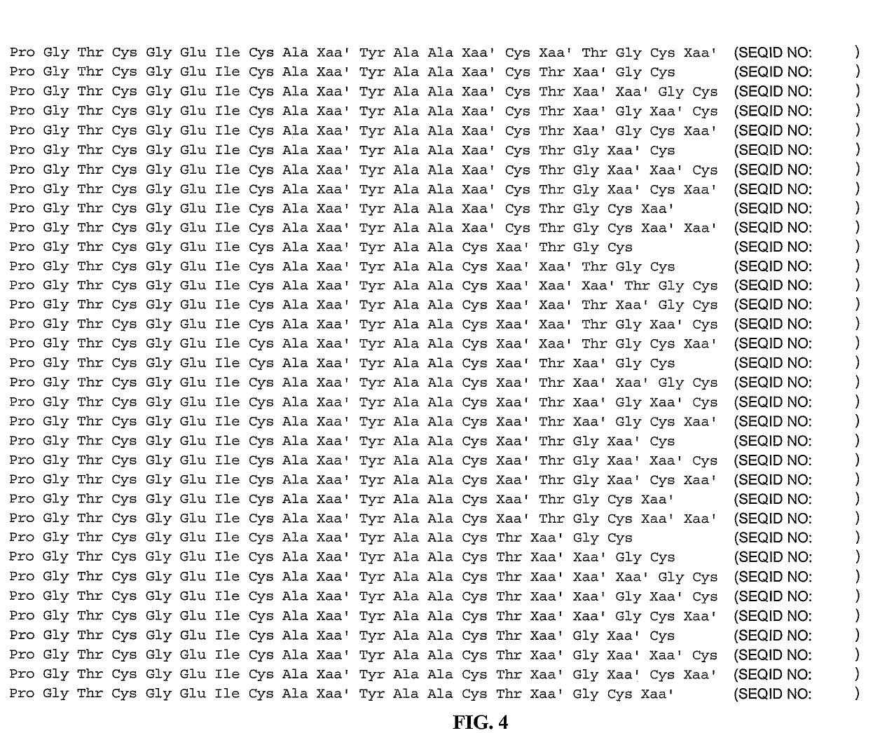

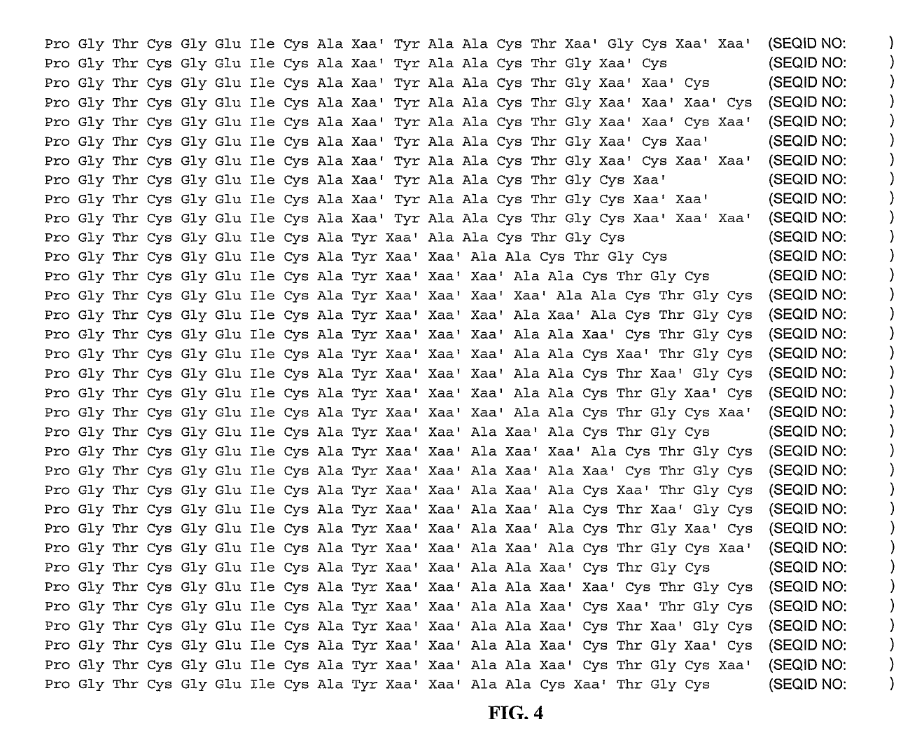

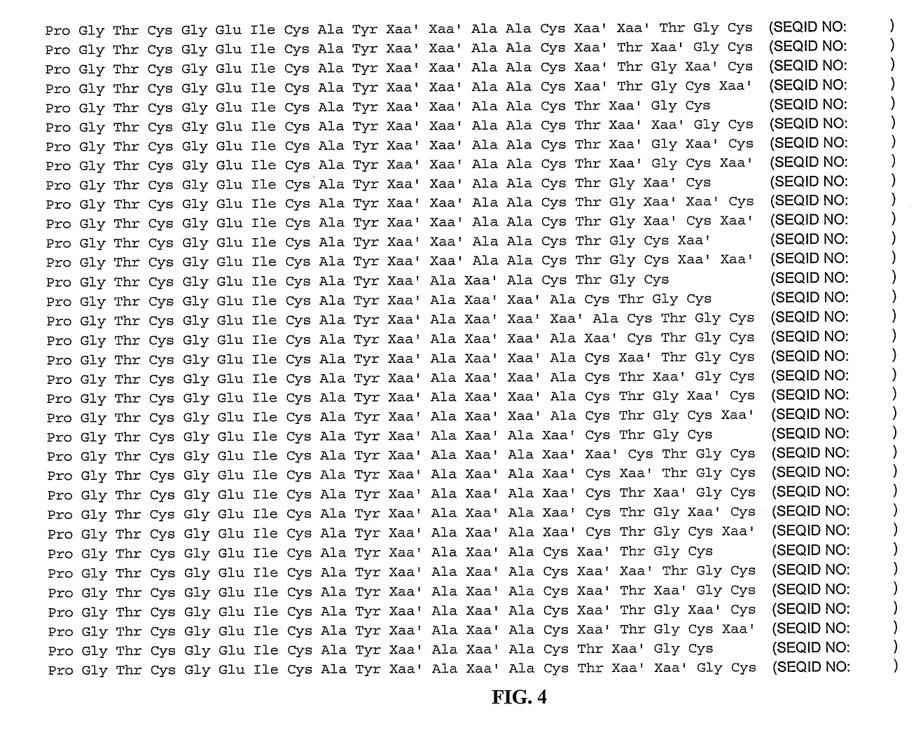

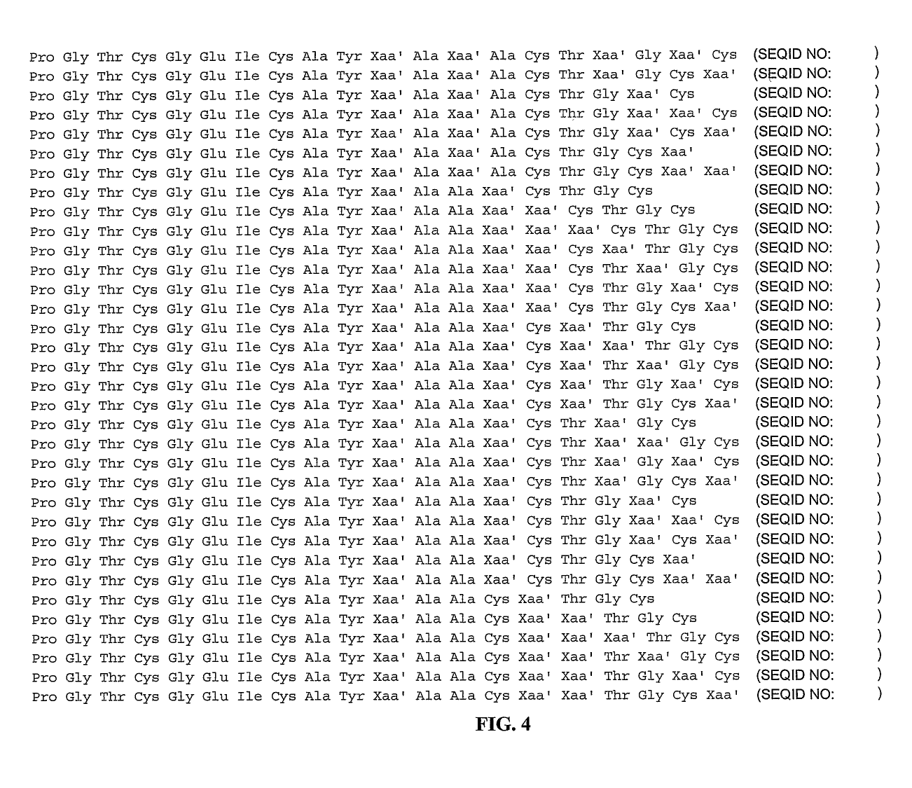

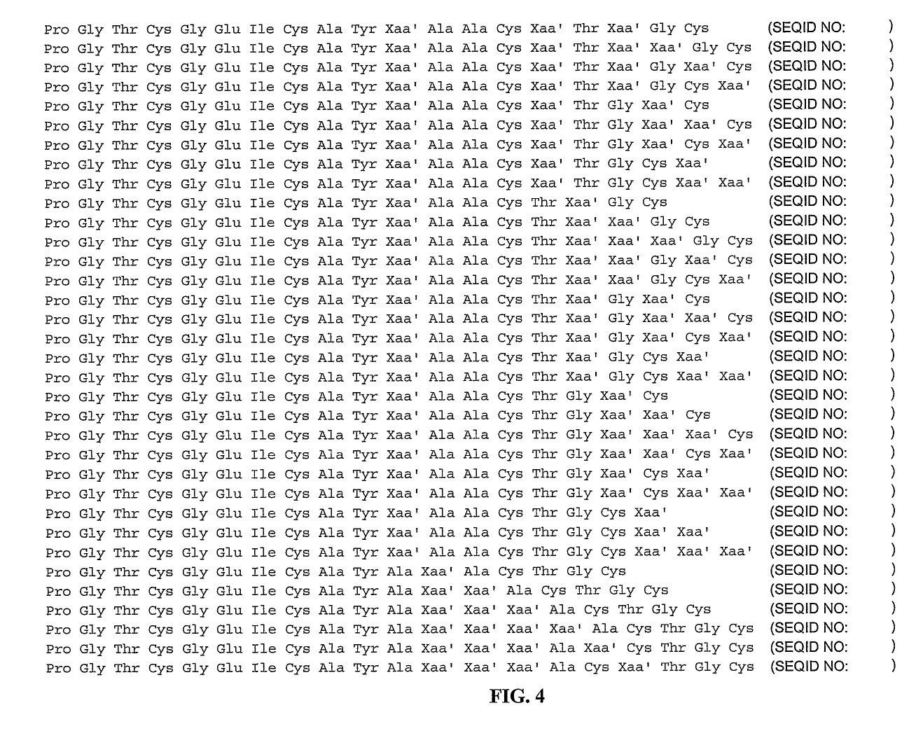

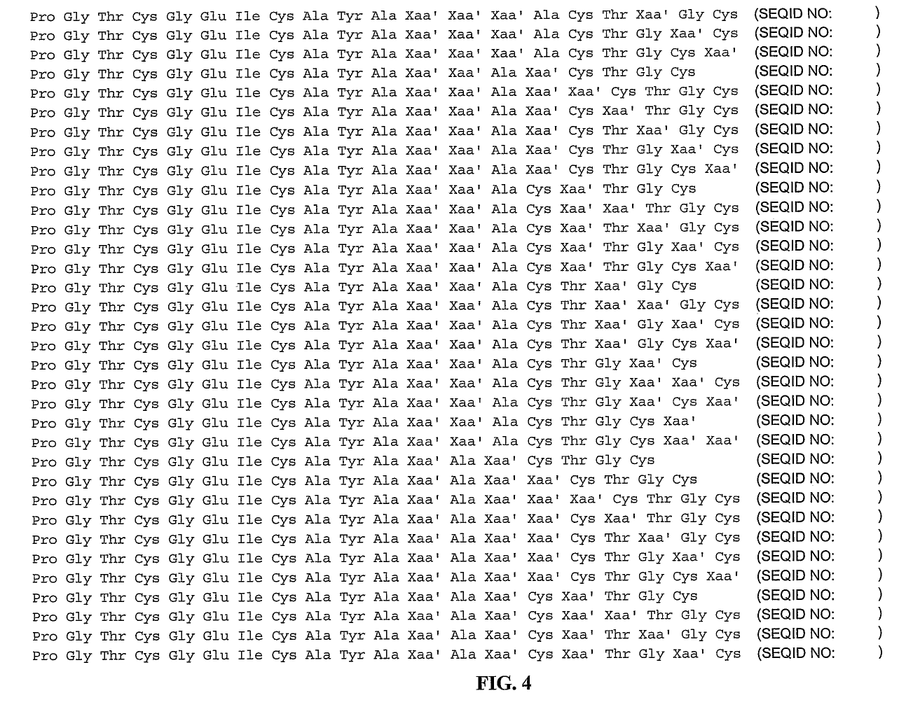

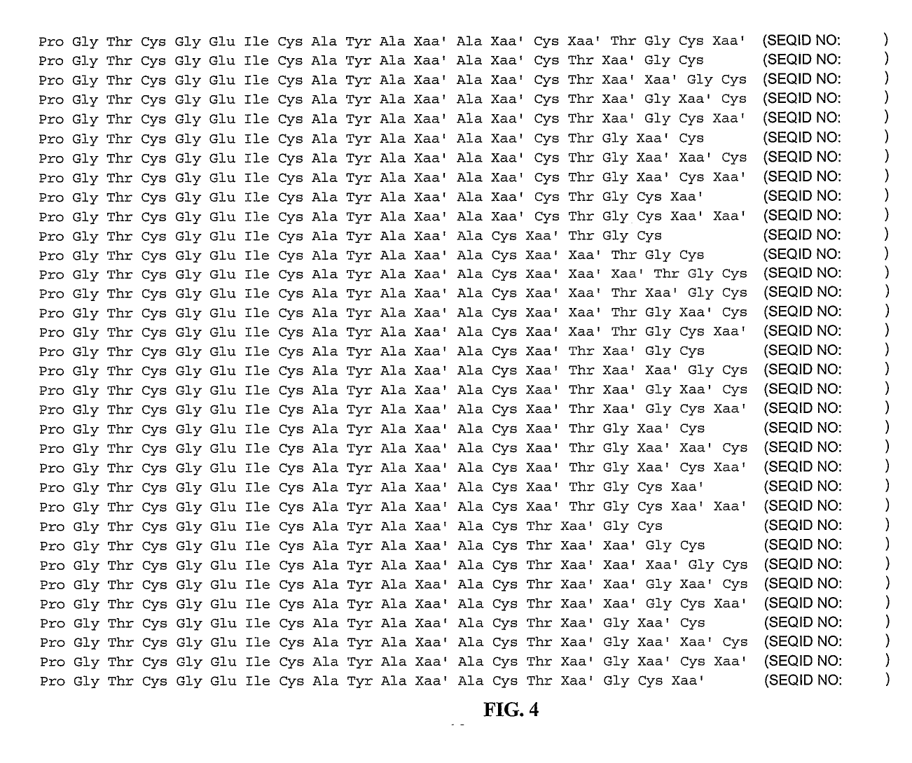

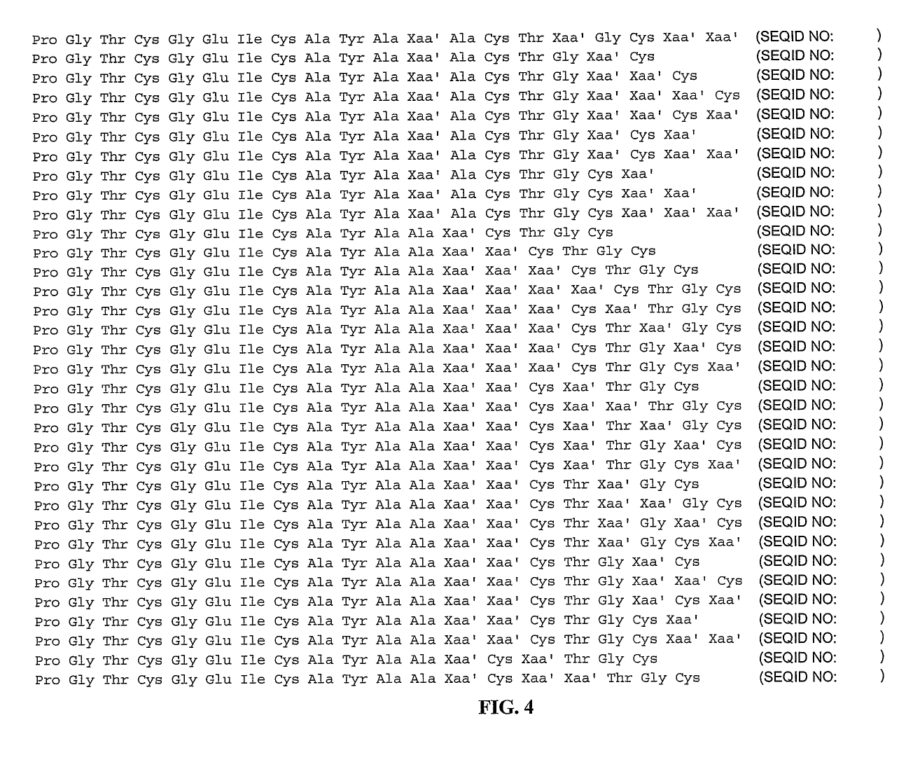

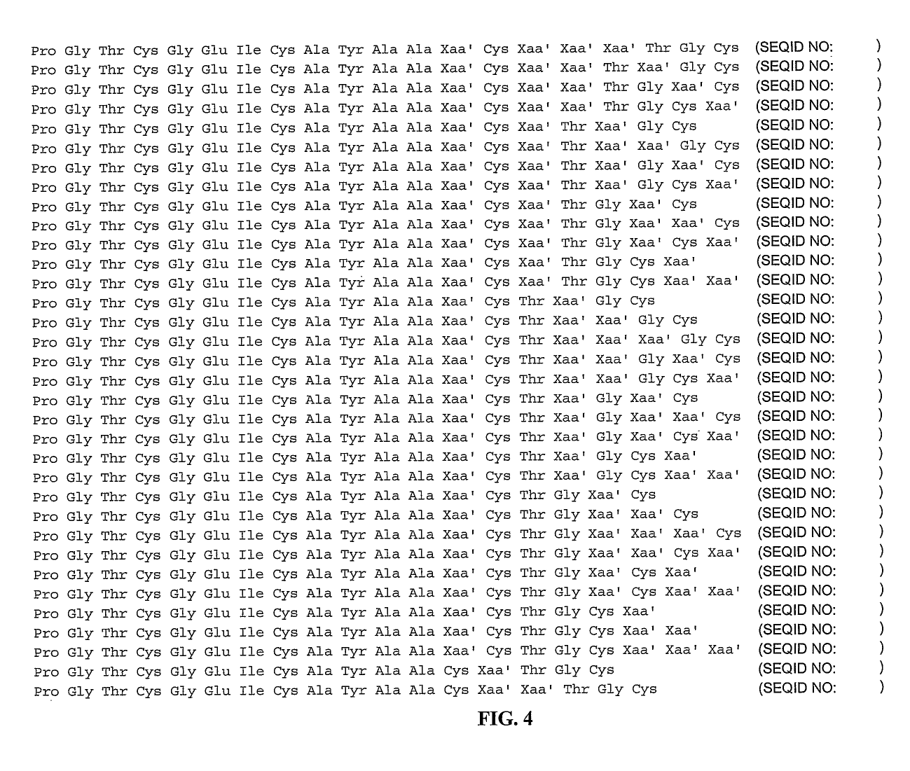

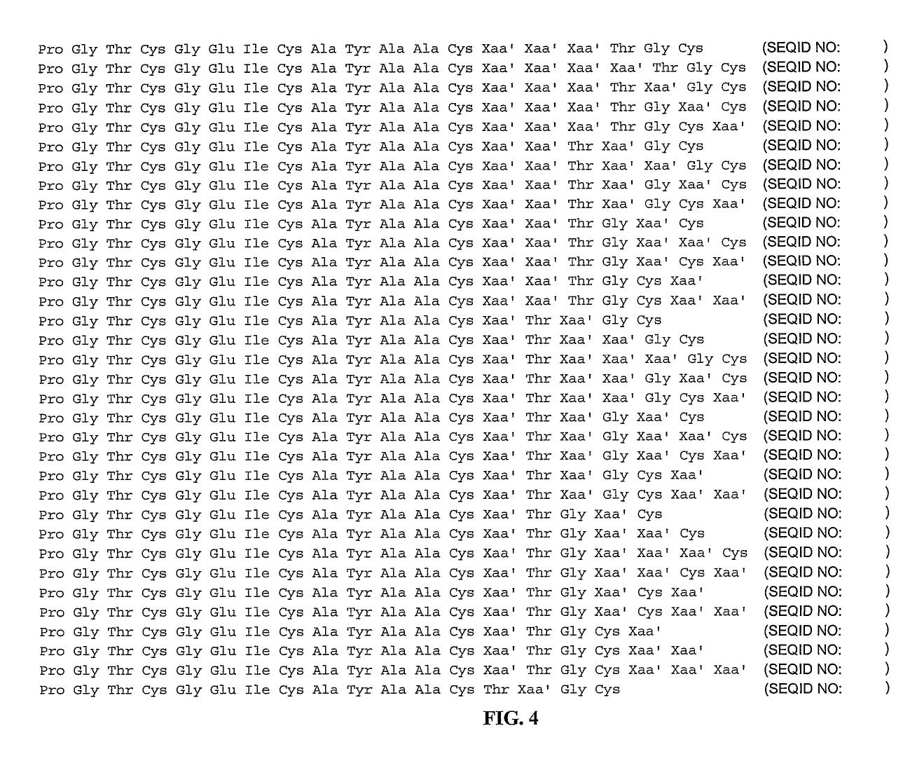

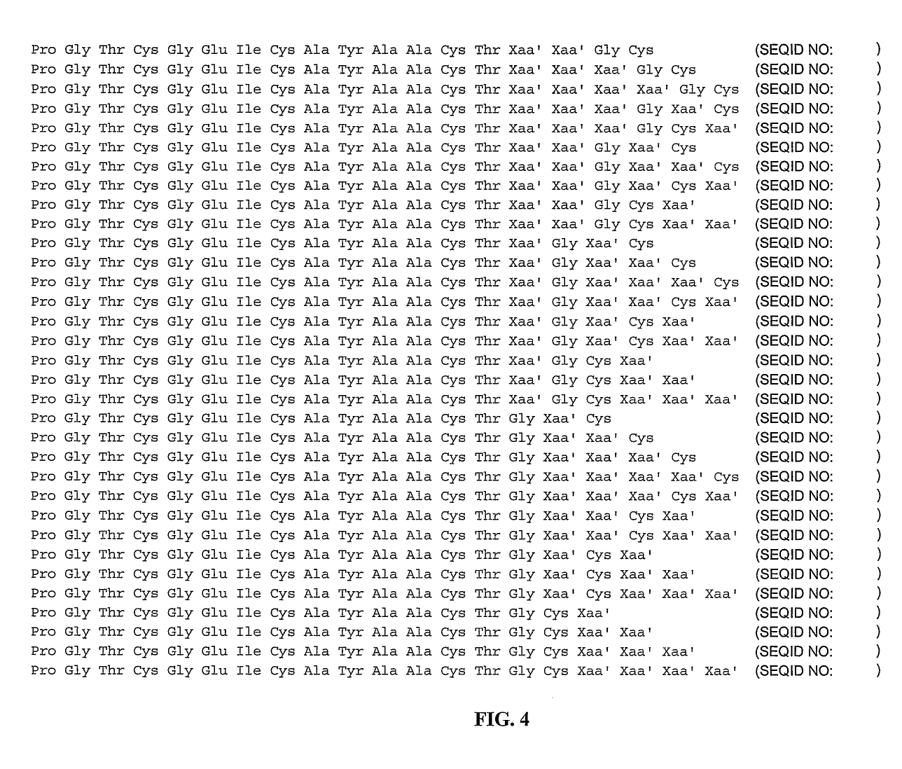

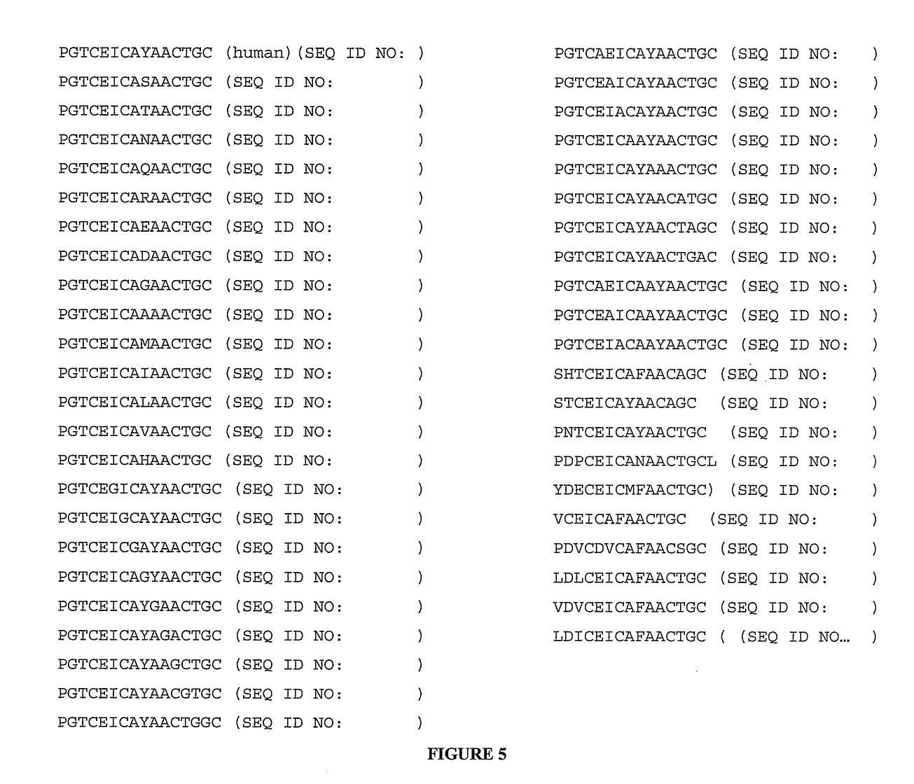

When it is desirable to potentiate the activity of guanylin by the administration of a chymotrypsin inhibitor it may also be desirable to administer guanylin or a biologically active variant or fragment thereof. A human guanylin is depicted in FIG. 5 along with various other guanylins. A number of guanylin variants are depicted in FIGS. 3 and 4.

### Activation of the Intestinal GC-C Receptor by Guanylin (T84 cGMP Assay)

The ability of guanylin to activate the intestinal GC-C receptor was assessed in an assay employing the T84 human colon carcinoma cell line (American Type Culture Collection (Bethesda, Md.). For the assays cells were grown to confluency in 24-well culture plates with a 1:1 mixture of Ham's F12 medium and Dulbecco's modified Eagle's medium (DMEM), supplemented with 5% fetal calf serum and were used at between passages 54 and 60.

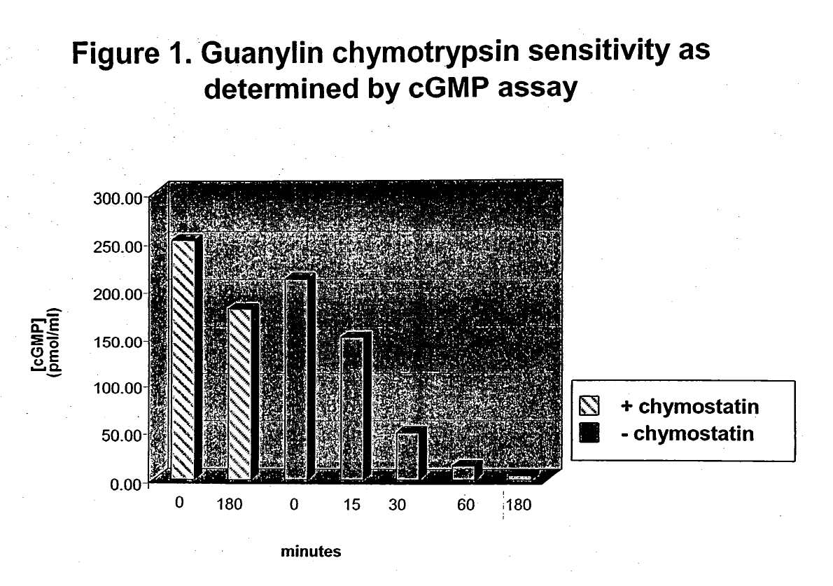

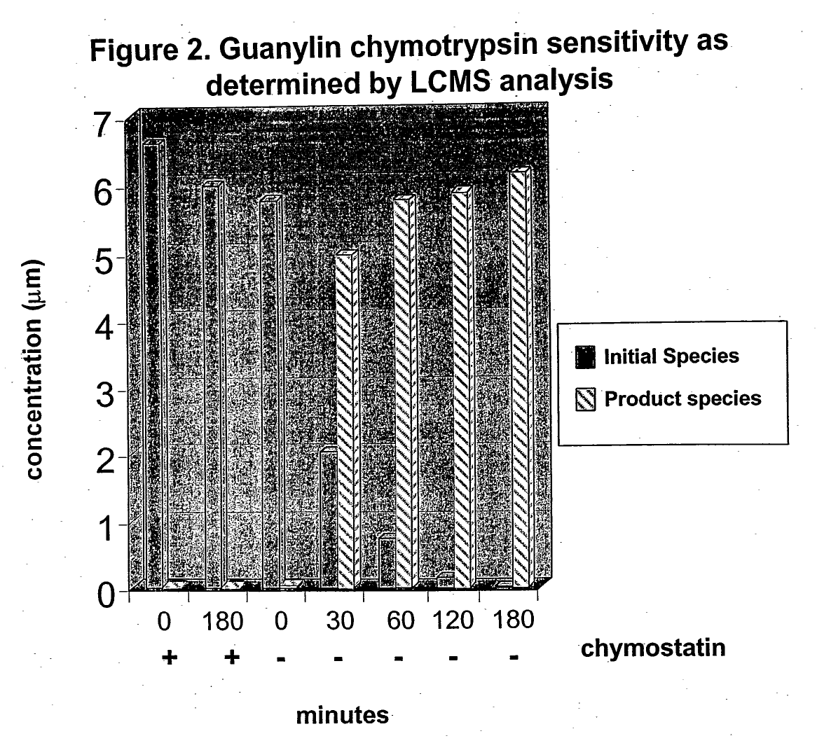

Briefly, monolayers of T84 cells in 24-well plates were washed twice with 1 ml/well DMEM, then incubated at 37° C. for 10 min with 0.45 ml DMEM containing 1 mM isobutylmethylxanthine (IBMX), a cyclic nucleotide phosphodiesterase inhibitor. Test peptides (50 μl) were then added and incubated for 30 minutes at 37° C. The media was aspirated and the reaction was then terminated by the addition of ice cold 0.5 ml of 0.1N HCl. The samples were held on ice for 20 minutes and then evaporated to dryness using a heat gun or vacuum centrifugation. The dried samples were resuspended in 0.5 ml of phosphate buffer provided in the Cayman Chemical Cyclic GMP EIA kit (Cayman Chemical, Ann Arbor, Mich.). Cyclic GMP was measured by EIA according to procedures outlined in the Cayman Chemical Cyclic GMP EIA kit. FIG. 1 shows that guanylin stimulated cGMP activity decreases over time in the presence of chymotrypsin. This activity was retained when the chymotrypsin inhibitor, chymostatin was present. FIG. 2 shows the results of LC/MS analysis of the processing of guanylin by chymotrypsin in the guanylin degradation assay.

### Intestinal GC-C Recetor Binding Assay

The ability of peptides and other agents to bind to the intestinal GC-C receptor can be tested as follows. Cells of the T84 human colon carcinoma cell line (American Type Culture Collection (Bethesda, Md.) are grown to confluence in 24-well culture plates with a 1:1 mixture of Ham's F12 medium and Dulbecco's modified Eagle's medium (DMEM), supplemented with 5% fetal calf serum. Cells used in the assay are typically between passages 54-60. Briefly, T84 cell monolayers in 24-well plates are washed twice with 1 ml of binding buffer (DMEM containing 0.05% bovine serum albumin and 25 mM HEPES, pH 7.2), then incubated for 30 min at 37° C. in the presence of mature radioactively labeled E. coli ST peptide and the test material at various concentrations. The cells are then washed four times with 1 ml of DMEM and solubilized with 0.5 ml/well IN NaOH. The level of radioactivity in the solubilized material is then determined using standard methods. In some cases, intestinal epithelial cell preparations may be used instead of T84 cells to assess receptor binding.

### Murine Gastrointestinal Transit (GIT) Assay

In order to determine if an agent of the invention has an effect on intestinal motility, the inhibitor can be tested in the murine gastrointestinal transit (GIT) assay (Moon et al. Infection and Immunity 25:127, 1979). This assay can also be used to determine the effect of an agent of the invention on intestinal motility. In this assay, charcoal, which can be readily visualized in the gastrointestinal tract is administered to mice after the administration of a test compound. The distance traveled by the charcoal is measured and expressed as a percentage of the total length of the colon.

Mice are fasted with free access to water for 12 to 16 hours before the treatment with peptide or control buffer. A test agent is orally administered in buffer (20 mM Tris pH 7.5) seven minutes before being given an oral dose of 5% Activated Carbon (Aldrich 242276-250G). Control mice are administered buffer only before being given a dose of Activated Carbon. After 15 minutes, the mice are sacrificed and their intestines from the stomach to the cecum are dissected. The total length of the intestine as well as the distance traveled from the stomach to the charcoal front is measured for each animal and the results are expressed as the percent of the total length of the intestine traveled by the charcoal front. Results are reported as the average of 10 mice±standard deviation. A comparison of the distance traveled by the charcoal between the mice treated with the agent versus the mice treated with vehicle alone is performed using a Student's t test and a statistically significant difference is considered for P<0.05. Positive controls for this assay may include Zelnorm®, a drug approved for IBS that is an agonist for the serotonin receptor 5HT4.

### Suckling Mouse Model of Intestinal Secretion (SuMi Assay)

Chymotrypsin inhibitors and other agents can be tested for their ability to increase intestinal secretion using a suckling mouse model of intestinal secretion. In this model a test compound is administered to suckling mice that are between seven and nine days old. After the mice are sacrificed, the gastrointestinal tract from the stomach to the cecum is dissected (“guts”). The remains (“carcass”) as well as the guts are weighed and the ratio of guts to carcass weight is calculated. If the ratio is above 0.09, one can conclude that the test compound increases intestinal secretion. Controls for this assay may include Zelnorm®

### Phenylbenzoquinone-Induced Writhing Model

The PBQ-induced writhing model can be used to assess whether administration of a compound such as a chymotrypsin inhibitor or a chymotrypsin inhibitor administered with guanylin or a biologically active variant or fragment thereofanalogue reduces pain. This model is described by Siegmund et al. (1957 Proc. Soc. Exp. Bio. Med. 95:729-731). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5^{th} to the 10^{th} minute after PBQ injection, and can also be counted between the 35^{th} and 40^{th} minute and between the 60^{th} and 65^{th} minute to provide a kinetic assessment. The results are expressed as the number of stretches and writhings (mean±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

### Colonic Hyperalgesia Animal Models

Hypersensitivity to colorectal distension is a common feature in patients with IBS and may be responsible for the major symptom of pain. Both inflammatory and non-inflammatory animal models of visceral hyperalgesia to distension have been developed to investigate the effect of compounds on visceral pain in IBS and can be used to assess the impact of a compound such as a chymotrypsin inhibitor or a chymotrypsin inhibitor administered with guanylin or biologically active variant or fragment thereof.

### I. Trinitrobenzenesulphonic acid (TNBS)-Induced Rectal Allodynia Model

Male Wistar rats (220-250 g) are premedicated with 0.5 mg/kg of acepromazine injected intraperitoneally (IP) and anesthetized by intramuscular administration of 100 mg/kg of ketamine. Pairs of nichrome wire electrodes (60 cm in length and 80 μm in diameter) are implanted in the striated muscle of the abdomen, 2 cm laterally from the white line. The free ends of electrodes are exteriorized on the back of the neck and protected by a plastic tube attached to the skin. Electromyographic (EMG) recordings are started 5 days after surgery. Electrical activity of abdominal striated muscle is recorded with an electroencephalograph machine (Mini VIII, Alvar, Paris, France) using a short time constant (0.03 sec.) to remove low-frequency signals (<3 Hz).

Ten days post surgical implantation, trinitrobenzenesulphonic acid (TNBS) is administered to induce rectal inflammation. TNBS (80 mg kg⁻¹ in 0.3 ml 50% ethanol) is administered intrarectally through a silicone rubber catheter introduced at 3 cm from the anus under light diethyl-ether anesthesia, as described (Morteau et al. 1994 Dig Dis Sci 39:1239). Following TNBS administration, rats are placed in plastic tunnels where they are severely limited in mobility for several days before colorectal distension (CRD). Experimental compound is administered one hour before CRDwhich is performed by insertion into the rectum, at 1 cm of the anus, a 4 cm long balloon made from a latex condom (Gue et al, 1997 Neurogastroenterol. Motil. 9:271). The balloon is fixed on a rigid catheter taken from an embolectomy probe (Fogarty). The catheter attached balloon is fixed at the base of the tail. The balloon, connected to a barostat is inflated progressively by step of 15 mmHg, from 0 to 60 mmHg, each step of inflation lasting 5 min. Evaluation of rectal sensitivity, as measured by EMG, is performed before (1-2 days) and 3 days following rectal instillation of TNBS.

The number of spike bursts that corresponds to abdominal contractions is determined per 5 min periods. Statistical analysis of the number of abdominal contractions and evaluation of the dose-effects relationships is performed by a one way analysis of variance (ANOVA) followed by a post-hoc (Student or Dunnett tests) and regression analysis for ED50 if appropriate.

### II. Stress-Induced Hyperalgesia Model

Male Wistar Rats (200-250 g) are surgically implanted with nichrome wire electrodes as in the TNBS model. Ten days post surgical implantation, partial restraint stress (PRS), is performed as described by Williams et al. for two hours (Williams et al. 1988 Gastroenterology 64:611). Briefly, under light anaesthesia with ethyl-ether, the foreshoulders, upper forelimbs and thoracic trunk are wrapped in a confining harness of paper tape to restrict, but not prevent body movements. Control sham-stress animals are anaesthetized but not wrapped. Thirty minutes before the end of the PRS session, the animals are administered test-compound or vehicle. Thirty minutes to one hour after PRS completion, the CRD distension procedure is performed as described above for the TNBS model with barostat at pressures of 15, 30, 45 and 60 mm Hg. Statistical analysis on the number of bursts is determined and analyzed as in the TNBS model above.

### Administration of Chymotrypsin Inhibitors

For treatment of gastrointestinal disorders, chymotrypsin inhibitors can be administered orally, e.g., as a tablet or cachet containing a predetermined amount of the active ingredient, pellet, gel, paste, syrup, bolus, electuary, slurry, capsule; “flash dosage”; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The chymotrypsin inhibitors can also be administered rectally, e.g., by suppository.

The chymotrypsin inhibitors can be co-administered with other agents used to treat gastrointestinal disorders including but not limited to those described herein. The chymotrypsin inhibitor can be administered together with guanylin or a guanylin variant or analogue. The inhibitors can also be administered by rectal suppository. For the treatment of disorders outside the gastrointestinal tract such as congestive heart failure and benign prostatic hypertrophy, inhibitors are preferably administered parenterally or orally. Chymotrypsin inhibitors preferably reach the small and/or large intestine in order to effectively reduce the activity of chymotrypsin that proteolytically digests guanylin. If the inhibitor(s) is to be administered orally, it is preferably formulated with an enteric coating. For example, the formulation can be provided with a non-porous, gastric acid-resistant polymer coating, e.g., a coating that is insoluble or only slightly soluble at pH 1.5 to pH 5, but is soluble above pH 5 or pH 5.5 up to or above pH 9. The polymer can include, for example, hydroxypropyl methyl cellulose phthalate, cellulose acetate phthalate, diethyl phthalate, dibutyl phthalate, and acrylic based polymers. The formulation can also be buffered by inclusion of a buffering agent, for example, sodium bicarbonate, potassium carbonate, potassium bicarbonate, ammonium carbonate, tromethamine, di(tris)hydroxymethylaminomethane) carbonate, tris-glycine, di-arginine, tri-arginine, poly-arginine, di-lysine, tri-lysine, poly-lysine, diethylamine and triethanolamine. It can be desirable for the buffering agent to provide a pH of from about 7 to about 9 in the small intestine or large intestine of a human patient. The formulation can also include a disintegrant, e.g., ursodiol, starch, modified starches, microcrystalline cellulose and propylene glycol alginate.

The chymotrypsin inhibitors described herein can be used alone or in combination with other agents. For example, they can be administered together with an agent for treating a gastrointestinal disorder. The chymotrypsin inhibitors can be administered in a combination therapy with guanylin or a biologically active variant or fragment thereof.

Combination therapy can be achieved by administering two or more agents, e.g., a chymotrypsin inhibitor and an agent for treating a gastrointestinal disorder or an analgesic peptide or compound, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation. The composition may contain other additives as needed, including for exanple lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, −103™ and −105™, sold by FMC Corporation, Marcus Hook, Pa., USA), or mixtures thereof,

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,

ANTI-CAKING AGENTS: calcium silicate, magnesium silicate, silicon dioxide, colloidal silicon dioxide, talc, or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, camauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(M-caprolactone) and poly(alkylene oxide) (U.S. 20030068384) to create a sustained release formulation. Such formulations can be used to implants that release a peptide or another agent over a period of a few days, a few weeks or several months depending on the polymer, the particle size of the polymer, and the size of the implant (see, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and US20020019446. In such sustained release formulations microparticles of peptide are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin. WO 03/053401 describes a formulation which may both enhance bioavailability and provide controlled releaseof the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), via intracavemosal injection, by transurethral application or by other routes.

The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115-124)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal adminisitration. The agents can be administered in the form a suppository or by other vaginal or rectal means. The agents can be administered in a transmembrane formulation as described in WO 90/07923. The agents can be administed non-invasively via the dehydrated particicles described in U.S. Pat. No. 6,485,706. The agent can be administered in an enteric-coated drug formulation as described in WO 02/49621. The agents can be administered intranassaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

Compositions for oral administration can be in the form a of a “flash dosage”, i.e., a solid dosage form that is administered orally, which rapidly disperses in the mouth, and hence does not require great effort in swallowing and allows the compound to be rapidly ingested or absorbed through the oral mucosal membranes. In some embodiments, suitable rapidly dispersing dosage forms are also used in other applications, including the treatment of wounds and other bodily insults and diseased states in which release of the medicament by externally supplied moisture is not possible.

“Flash dose” forms are known in the art; see for example, effervescent dosage forms and quick release coatings of insoluble microparticles in U.S. Pat. Nos. 5,578,322 and 4,607,697; freeze dried foams and liquids in U.S. Pat. Nos. 4,642,903 and 5,631,023; melt spinning of dosage forms in U.S. Pat. Nos. 4,855,326; 5,380,326; and 5,518,730; solid, freeform fabrication in U.S. Pat. No. 6,471,992; saccharide-based carrier matrix and a liquid binder in U.S. Pat. Nos. 5,587,172; 5,616,344; 6,277,406; and 5,622,719; and other forms known to the art.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluroalkanes (HFAs, i.e. HFA-134a and HFA-227, or a mixture thereof), dichlorodifluoromethane (or other chlorofluocarbon propellants such as a mixture of Propellants 11,12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccaride are advantageous in that some of them also reportedly enhance absorption of the peptide in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are desribed in EP 1338272A1 WO 90/09781, U.S. Pat. No. 5,348,730, U.S. Pat. No. 6,436,367, WO 91/04011, and U.S. Pst. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means. The agent can be fused to immunoglobulins or albumin, or incorporated into a lipsome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. Peptides can also be modified with alkyl groups (e.g., C1-C20 straight or branched alkyl groups); fatty acid radicals; and combinations of PEG, alkyl groups and fatty acid radicals (see U.S. Pat. No. 6,309,633; Soltero et al., 2001 Innovations in Pharmaceutical Technology 106-110). The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences (18th Edition, Mack Publishing Company, 1995).

The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

Methods to increase chemical and/or physical stability of the agents the described herein are found in U.S. Pat. No. 6,541,606, U.S. Pat. No. 6,068,850, U.S. Pat. No. 6,124,261, U.S. Pat. No. 5,904,935, and WO 00/15224, U.S. 20030069182 (via the addition of nicotinamide), U.S. 20030175230A1, U.S. 20030175230A1, U.S. 20030175239A1, U.S. 20020045582, U.S. 20010031726, WO 02/26248, WO 03/014304, WO 98/00152A1, WO 98/00157A1, WO 90/12029, WO 00/04880, and WO 91/04743, WO 97/04796 and the references cited therein.

Methods to increase bioavailability of the agents described herein are found in U.S. Pat. No. 6,008,187, U.S. Pat. No. 5,424,289, U.S. 20030198619, WO 90/01329, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract.

The agents described herein can be fused to a modified version of the blood serum protein transferrin. U.S. 20030221201, U.S. 20040023334, U.S. 20030226155, WO 04/020454, and WO 04/019872 discuss the manufacture and use of transferrin fusion proteins. Transferrin fusion proteins may improve circulatory half life and efficacy, decrease undesirable side effects and allow reduced dosage.

### Combitherapy

### Analgesic Agents in Combitherapy

The chymotrypsin inhibitors described herein can be used in combination therapy with an analgesic agent, e.g., an analgesic compound or an analgesic peptide. These peptides and compounds can be administered with the agents of the invention (simultaneously or sequentially). They can also be optionally covalently linked or attached to an agent described herein to create therapeutic conjugates. Among the useful analgesic agents are: Ca channel blockers, 5HT receptor antagonists (for example 5HT3, 5HT4 and 5HT1 receptor antagonists), opioid receptor agonists (loperamide, fedotozine, and fentanyl), NK1 receptor antagonists, CCK receptor agonists (e.g., loxiglumide), NK1 receptor antagonists, NK3 receptor antagonists, norepinephrine-serotonin reuptake inhibitors (NSRI), vanilloid and cannabanoid receptor agonists, and sialorphin. Analgesics agents in the various classes are described in the literature.

Among the useful analgesic peptides are sialorphin-related peptides, including those comprising the amino acid sequence QHNPR (SEQ ID NO: ), including: VQHNPR (SEQ ID NO: ); VRQHNPR (SEQ ID NO: ); VRGQHNPR (SEQ ID NO: ); VRGPQHNPR (SEQ ID NO: ); VRGPRQHNPR (SEQ ID NO: ); VRGPRRQHNPR (SEQ ID NO: ); and RQHNPR (SEQ ID NO: ). Sialorphin-related peptides bind to neprilysin and inhibit neprilysin-mediated breakdown of substance P and Met-enkephalin. Thus, compounds or peptides that are inhibitors of neprilysin are useful analgesic agents which can be administered with the peptides of the invention in a co-therapy or linked to the peptides of the invention, e.g., by a covalent bond. Sialophin and related peptides are described in U.S. Pat. No. 6,589,750; U.S. 20030078200 A1; and WO 02/051435 A2.

Opioid receptor antagonists and agonists can be administered with the agents of the invention in co-therapy or linked to the agent of the invention, e.g., by a covalent bond. For example, opioid receptor antagonists such as naloxone, naltrexone, methyl nalozone, nalmefene, cypridime, beta funaltrexamine, naloxonazine, naltrindole, and nor-binaltorphimine are thought to be useful in the treatment of IBS. It can be useful to formulate opioid antagonists of this type is a delayed and sustained release formulation such that initial release of the antagonist is in the mid to distal small intestine and/or ascending colon. Such antagonists are described in WO 01/32180 A2. Enkephalin pentapeptide (HOE825; Tyr-D-Lys-Gly-Phe-L-homoserine) is an agonist of the mu and delta opioid receptors and is thought to be useful for increasing intestinal motility (Eur. J Pharm. 219:445, 1992), and this peptide can be used in conjunction with the peptides of the invention. Also useful is trimebutine which is thought to bind to mu/delta/kappa opioid receptors and activate release of motilin and modulate the release of gastrin, vasoactive intestinal peptide, gastrin and glucagons. Kappa opioid receptor agonists such as fedotozine, asimadoline, and ketocyclazocine, and compounds described in WO 03/097051 A2 can be used with or linked to the agents of the invention. In addition, mu opioid receptor agonists such as morphine, diphenyloxylate, frakefamide (H-Tyr-D-Ala-Phe(F)-Phe-NH₂; WO 01/019849 A1) and loperamide can be used.

Tyr-Arg (kyotorphin) is a dipeptide that acts by stimulating the release of metenkephalins to elicit an analgesic effect (J. Biol. Chem 262:8165, 1987). Kyotorphin can be used with or linked to the agents of the invention.

Chromogranin-derived peptide (CgA 47-66; see, e.g., Ghia et al. 2004 Regulatory Peptides 119:199) can be used with or linked to the agents of the invention.

CCK receptor agonists such as caerulein from amphibians and other species are useful analgesic agents that can be used with or linked to the agents of the invention.

Conotoxin peptides represent a large class of analgesic peptides that act at voltage gated Ca channels, NMDA receptors or nicotinic receptors. These peptides can be used with or linked to the agents of the invention.

Peptide analogs of thymulin (FR Application 2830451) can have analgesic activity and can be used with or linked to the agents of the invention.

CCK (CCKa or CCKb) receptor antagonists, including loxiglumide and dexloxiglumide (the R-isomer of loxiglumide) (WO 88/05774) can have analgesic activity and can be used with or linked to the peptides of the invention.

Other useful analgesic agents include 5-HT4 agonists such as tegaserod/zelnorm and lirexapride. Such agonists are described in: EP1321142 A1, WO 03/053432A1, EP 505322 A1, EP 505322 B1, U.S. Pat. No. 5,510,353, EP 507672 A1, EP 507672 B1, and U.S. Pat. No. 5,273,983.

Calcium channel blockers such as ziconotide and related compounds described in, for example, EP625162B1, U.S. Pat. No. 5,364,842, U.S. Pat. No. 5,587,454, U.S. Pat. No. 5,824,645, U.S. Pat. No. 5,859,186, U.S. Pat. No. 5,994,305, U.S. Pat. No. 6,087,091, U.S. Pat. No. 6,136,786, WO 93/13128 A1, EP 1336409 A1, EP 835126 A1, EP 835126 B1, U.S. Pat. No. 5,795,864, U.S. Pat. No. 5,891,849, U.S. Pat. No. 6,054,429, WO 97/01351 A1, can be used with or linked to the agents of the invention.

Various antagonists of the NK-1, NK-2, and NK-3 receptors (for a review see Giardina et al. 2003 Drugs 6:758) can be can be used with or linked to the agents of the invention.

NK1 receptor antagonists such as: aprepitant (Merck & Co Inc), vofopitant, ezlopitant (Pfizer, Inc.), R-673 (Hoffmann-La Roche Ltd), SR-14033 and related compounds described in, for example, EP 873753 A1, US 20010006972 A1, U.S. 20030109417 A1, WO 01/52844 A1, can be used with or linked to the agents of the invention.

NK-2 receptor antagonists such as nepadutant (Menarini Ricerche SpA), saredutant (Sanofi-Synthelabo), SR-144190 (Sanofi-Synthelabo) and UK-290795 (Pfizer Inc) can be used with or linked to the agents of the invention.

NK3 receptor antagonists such as osanetant (SR-142801; Sanofi-Synthelabo), SSR-241586 talnetant and related compounds described in, for example, WO 02/094187 A2, EP 876347 A1, WO 97/21680 A1, U.S. Pat. No. 6,277,862, WO 98/11090, WO 95/28418, WO 97/19927, and Boden et al. (J Med Chem. 39:1664-75, 1996) can be used with or linked to the agents of the invention.

Norepinephrine-serotonin reuptake inhibitors such as milnacipran and related compounds described in WO 03/077897 A1 can be used with or linked to the agents of the invention.

Vanilloid receptor antagonists such as arvanil and related compounds described in WO 01/64212 A1 can be used with or linked to the peptides of the invention.

Where the analgesic is a peptide and is covalently linked to an agent described herein the resulting peptide may also include at least one trypsin cleavage site. When present within the peptide, the analgesic peptide may be preceded by (if it is at the carboxy terminus) or followed by (if it is at the amino terminus) a trypsin cleavage site that allows release of the analgesic peptide.

In addition to sialorphin-related peptides, analgesic peptides include: AspPhe, endomorphin-1, endomorphin-2, nocistatin, dalargin, lupron, zicnotide, and substance P.

### Other Agents for Use in Combitherapy

Also within the invention are pharmaceutical compositions comprising a chymotrypsin inhibitor and a second therapeutic agent. The second therapeutic agent can be administered to treat any condition for which it is useful, including conditions that are not considered to be the primary indication for treatment with the second therapeutic agent.

Examples of additional therapeutic agents to treat gastrointestinal and other disorders include:
(1) agents to treat constipation (e.g., a chloride channel activator such as SPI-0211; Sucampo Pharmaceuticals, Inc.; Bethesda, Md., a laxative such as MiraLax; Braintree Laboratories, Braintree Mass.);
(2) acid reducing agents such as proton pump inhibitors (e.g., omeprazole, esomeprazole, lansoprazole, pantorazole and rabeprazole) and Histamine H2-receptor antagonist (also known as H2 receptor blockers including cimetidine, ranitidine, famotidine and nizatidine);
(3) prokinetic agents including metoclopramide (Reglan®), domperidone (Motilium®), erythromycin or cisapride (propulsid®)
(4) pro-motility agents such as the vasostatin-derived peptide, chromogranin A (4-16) (see, e.g., Ghia et al. 2004 Regulatory Peptides 121:31) or motilin agonists (e.g., GM-611 or mitemcinal fumarate);
(5) complete or partial 5HT (e.g. 5HT1, 5HT2, 5HT3, 5HT4) receptor agonists or antagonists (including 5HT4 receptor agonists such as tegaserod (ZELNORM®), mosapride and renzapride; 5HT3 receptor agonists such as MKC-733; and 5HT3 receptor antagonists such as alosetron and ATI-7000 (Aryx Therapeutics, Santa Clara Calif.);
(6) muscarinic receptor agonists;
(7) anti-inflammatory agents;
(8) antispasmodics;
(9) antidepressants;
(10) centrally-acting analgesic agents such as opioid receptor agonistsopioid receptor antagonists (e.g., naltrexone);
(11) agents for the treatment of Inflammatory bowel disease;
(12) agents for the treatment of Crohn's disease and/or ulcerative colitis (e.g., alequel (Enzo Biochem, Inc.; Farmingsale, N.Y.), the anti-inflammatory peptide RDP58 (Genzyme, Inc.; Cambridge, Mass.), and TRAFICET-EN™ (ChemoCentryx, Inc.; San Carlos, Calif.);
(13) agents that treat gastrointestinal or visceral pain;
(14) PDE (phosphodiesterase) inhibitors including but not limited to those disclosed in U.S. Pat. No. 6,331,543 and cilomilast, cilostamide, denbufyllene piclamilast, filaminast, milrinone, pentoxifylline, roflumilast, rolipram, sildenafil(Viagra®), tadalafil(Cialis®), theophylline, vardenafil(Levitra®), zaprinast, and zardaverine.
(15) purgatives that draw fluids to the intestine (e.g., VISICOL®, a combination of sodium phosphate monobasic monohydrate and sodium phosphate dibasic anhydrate);
(16) Corticotropin Releasing Factor (CRF) receptor antagonists (NBI-34041; Neurocrine Biosciences, San Diego, Calif.);
(17) glucagon-like peptides (glp-1) and analogues thereof (including exendin-4) and inhibitors of DPP-IV (DPP-IV mediates the inactivation of glp-1); and
(18) the probiotic PROBACTRIX® (The BioBalance Corporation; New York, NY) which contains microorganisms useful in the treatment of gastrointestinal disorders.

The agents of the invention can be used in combination therapy with insulin and related compounds including primate, rodent, or rabbit insulin including biologically active variants thereof including allelic variants, more preferably human insulin available in recombinant form. Sources of human insulin include pharmaceutically acceptable and sterile formulations such as those available from Eli Lilly (Indianapolis, Ind. 46285) as Humulin.™. (human insulin rDNA origin). See the THE PHYSICIAN'S DESK REFERENCE, 55.sup.th Ed. (2001) Medical Economics, Thomson Healthcare (disclosing other suitable human insulins). The agents of the invention can also be used in combination therapy with agents that can boost insulin effects or levels of a subject upon administration, e.g. glipizide and/or rosiglitazone.

The agents of the invention can also be used in combination with agents such a tianeptine (STABLON®) and other agents described in U.S. Pat. No. 6,683,072; (E)-4 (1,3bis(cyclohexylmethyl)-1,2,34,-tetrahydro-2,6-diono-9H-purin-8-yl)cinnamic acid nonaethylene glycol methyl ether ester and related compounds described in WO 02/067942.

The agents of the invention can also be used in combination therapy with agents (e.g., aldolor) for the treatment of postoperative ileus.

The agents of the invention can be used in combination therapy with an anti-hypertensive agent including but not limited to:
(1) diuretics, such as thiazides, including chlorthalidone, chlorthiazide, dichlorophenamide, hydroflumethiazide, indapamide, polythiazide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like;
(2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like;
(3) calcium channel blockers such as amlodipine, aranidipine, azelnidipine, bamidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like;
(4) angiotensin converting enzyme (ACE) inhibitors such as benazepril; captopril; ceranapril; cilazapril; delapril; enalapril; enalopril; fosinopril; imidapril; lisinopril; losinopril; moexipril; quinapril; quinaprilat; ramipril; perindopril; perindropril; quanipril; spirapril; tenocapril; trandolapril, and zofenopril, and the like;
(5) neutral endopeptidase inhibitors such as omapatrilat, cadoxatril and ecadotril, fosidotril, sampatrilat, AVE7688, ER4030, and the like;
(6) endothelin antagonists such as tezosentan, A308165, and YM62899, and the like;
(7) vasodilators such as hydralazine, clonidine, minoxidil, and nicotinyl alcohol, and the like;
(8) angiotensin II receptor antagonists such as aprosartan, candesartan, eprosartan, irbesartan, losartan, olmesartan, pratosartan, tasosartan, telmisartan, valsartan, and EXP-3137, F16828K, and RNH6270, and the like;
(9) α/β adrenergic blockers such as nipradilol, arotinolol and amosulalol, and the like;
(10) alpha 1 blockers, such as terazosin, urapidil, prazosin, tamsulosin, bunazosin, trimazosin, doxazosin, naftopidil, indoramin, WHP 164, and XEN010, and the like;
(11) alpha 2 agonists such as lofexidine, tiamenidine, moxonidine, rilmenidine and guanobenz, and the like;
(12) aldosterone inhibitors, and the like; and
(13) angiopoietin-2-binding agents such as those disclosed in WO03/030833.

The agents of the invention can be used in combination therapy with one or more of the following agents useful in the treatment of respiratory and other disorders:
(1) β-agonists including but not limited to: albuterol (PROVENTIL®, SALBUTAMO1®, VENTOLIN®), bambuterol, bitoterol, clenbuterol, fenoterol, formoterol, isoetharine (BRONKOSOL®), BRONKOMETER®), metaproterenol (ALUPENT®, METAPREL®), pirbuterol (MAXAIR®), reproterol, rimiterol, salmeterol, terbutaline (BRETHAIRE(®, BRETHINE®, BRICANYL®), adrenalin, isoproterenol (ISUPREL®), epinephrine bitartrate (PRIMATENE®), ephedrine, orciprenline, fenoterol and isoetharine;
(2) steroids, including but not limited to beclomethasone, beclomethasone dipropionate, betamethasone, budesonide, bunedoside, butixocort, dexamethasone, flunisolide, fluocortin, fluticasone, hydrocortisone, methyl prednisone, mometasone, predonisolone, predonisone, tipredane, tixocortal, triamcinolone, and triamcinolone acetonide;
(3) β2-agonist-corticosteroid combinations [e.g., salmeterol-fluticasone (ADVAIR®), formoterol-budesonid (SYMBICORT®)];
(4) leukotriene D4 receptor antagonists/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (SINGULAIR®), pranlukast, iralukast, pobilukast, SKB-106,203 and compounds described as having LTD4 antagonizing activity described in U.S. Pat. No. 5,565,473;
(5) 5-lipoxygenase inhibitors and/or leukotriene biosynthesis inhibitors [e.g., zileuton and BAY1005 (CA registry 128253-31-6)];
(6) histamine H1 receptor antagonists/antihistamines (i.e., any compound that is capable of blocking, inhibiting, reducing or otherwise interrupting the interaction between histamine and its receptor) including but not limited to: astemizole, acrivastine, antazoline, azatadine, azelastine, astamizole, bromopheniramine, bromopheniramine maleate, carbinoxamine, carebastine, cetirizine, chlorpheniramine, chloropheniramine maleate, cimetidine ,clemastine, cyclizine, cyproheptadine, descarboethoxyloratadine, dexchlorpheniramine, dimethindene, diphenhydramine, diphenylpyraline, doxylamine succinate, doxylamine, ebastine, efletirizine, epinastine, famotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norasternizole, noraztemizole, phenindamine, pheniramine, picumast, promethazine, pynlamine, pyrilamine, ranitidine, temelastine, terfenadine, trimeprazine, tripelenamine, and triprolidine;
(7) an anticholinergic including but not limited to: atropine, benztropine, biperiden, flutropium, hyoscyamine, ilutropium, ipratropium, ipratropium bromide, methscopolamine, oxybutinin, rispenzepine, scopolamine, and tiotropium;
(8) an anti-tussive including but not limited to: dextromethorphan, codeine, and hydromorphone;
(9) a decongestant including but not limited to: pseudoephedrine and phenylpropanolamine;
(10) an expectorant including but not limited to: guafenesin, guaicolsulfate, terpin, ammonium chloride, glycerol guaicolate, and iodinated glycerol;
(11) a bronchodilator including but not limited to: theophylline and aminophylline;
(12) an anti-inflammatory including but not limited to: fluribiprofen, diclophenac, indomethacin, ketoprofen, S-ketroprophen, tenoxicam;
(13) a PDE inhibitor including but not limited to those disclosed in U.S. Pat. No. 6,331,543 and cilomilast, cilostamide, denbufyllene piclamilast, filaminast, milrinone, pentoxifylline, roflumilast, rolipram, sildenafil(Viagra®), tadalafil(Cialis(®), theophylline, vardenafil(Levitra®), zaprinast, and zardaverine;
(14) a recombinant humanized monoclonal antibody [e.g. xolair (also called omalizumab), rhuMab, and talizumab];
(15) a humanized lung surfactant including recombinant forms of surfactant proteins SP-B, SP-C or SP-D [e.g. SURFAXIN®, formerly known as dsc-104 (Discovery Laboratories)],
(16) agents that inhibit epithelial sodium channels (ENaC) such as amiloride and related compounds;
(17) antimicrobial agents used to treat pulmonary infections such as acyclovir, amikacin, amoxicillin, doxycycline, trimethoprin sulfamethoxazole, amphotericin B, azithromycin, clarithromycin, roxithromycin, clarithromycin, cephalosporins( ceffoxitin, cefmetazole etc), ciprofloxacin, ethambutol, gentimycin, ganciclovir, imipenem, isoniazid, itraconazole, penicillin, ribavirin, rifampin, rifabutin,amantadine, rimantidine, streptomycin, tobramycin, and vancomycin;
(18) agents that activate chloride secretion through Ca++ dependent chloride channels (such as purinergic receptor (P2Y(2) agonists);
(19) agents that decrease sputum viscosity, such as human recombinant DNase 1, (Pulmozyrne®);
(20) nonsteroidal anti-inflammatory agents (acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam,tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac); and
(21) aerosolized antioxidant therapeutics such as S-Nitrosoglutathione.

The agents of the invention can be used in combination therapy with an anti-obesity agent including but not limited to:
(1) 11βHSD-1 (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3α-decahydro-1,2,4-triazolo[4,3-α][11]annulene, and those compounds disclosed in WO01/90091, WO01/90090, WO01/90092 and WO02/072084;
(2) 5HT (serotonin) transporter inhibitors, such as paroxetine, fluoxetine, fenfluramine, fluvoxamine, sertraline, and imipramine, and those disclosed in WO03/00663;
(3) 5HT antagonists such as those in WO03/037871, WO03/037887, and the like;
(4) 5HT1a modulators such as those disclosed in WO03/031439, and the like;
(5) 5HT-2 agonists;
(6) 5HT2c (serotonin receptor 2c) agonists, such as BVT933, DPCA37215, IK264, PNU 22394, WAY161503, R-1065, and YM 348 and those disclosed in U.S. Pat. No. 3,914,250 and PCT publication Nos. WO02/36596, WO02/48124, WO02/10169, WO01/66548, WO02/44152, WO02/51844, WO02/40456, and WO02/40457;
(7) 5HT6 receptor modulators, such as those in WO03/030901, WO03/035061, WO03/039547, and the like;
(8) ACC2 (acetyl-CoA carboxylase-2) inhibitors;
(9) acyl-estrogens, such as oleoyl-estrone, disclosed in del Mar-Grasa, M. et al., Obesity Research, 9:202-9 (2001) and Japanese Patent Application No. JP 2000256190;
(10) alpha-lipoic acid (alpha-LA);
(11) anorectic bicyclic compounds such as 1426 (Aventis) and 1954 (Aventis), and the compounds disclosed in WO00/18749, WO01/32638, WO01/62746, WO01/62747, and WO03/015769;
(12) AOD9604;
(13) appetite suppressants such as those in WO03/40107;
(14) ATL-962 (Alizyme PLC);
(15) benzocaine;
(16) benzphetamine hydrochloride (Didrex);
(17) bladderwrack (focus vesiculosus);
(18) BRS3 (bombesin receptor subtype 3) agonists;
(19) bupropion;
(20) caffeine;
(21) CB 1 (cannabinoid-1 receptor) antagonist/inverse agonists, such as rimonabant (Acomplia; Sanofi Synthelabo), SR-147778 (Sanofi Synthelabo), BAY 65-2520 (Bayer), and SLV 319 (Solvay), and those disclosed in U.S. Pat. Nos. 4,973,587, 5,013,837, 5,081,122, 5,112,820, 5,292,736, 5,532,237, 5,624,941, 6,028,084, and 6,509,367 and WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and U.S. Pat. No. 6,509,367 and EPO Application No. EP-658546;
(22) CCK agonists;
(23) CCK-A (cholecystokinin-A) agonists, such as AR-R 15849, GI 181771, JMV-180, A-71378, A-71623 and SR146131, and those discribed in U.S. Pat. No. 5,739,106;
(24) chitosan;
(25) chromium;
(26) CNTF (Ciliary neurotrophic factors), such as GI-181771 (Glaxo-SmithKline), SR146131 (Sanofi Synthelabo), butabindide, PD170,292, and PD 149164 (Pfizer);
(27) CNTF derivatives, such as axokine (Regeneron), and those disclosed in PCT Application Nos. WO 94/09134, WO 98/22128, and WO 99/43813;
(28) conjugated linoleic acid;
(29) corticotropin-releasing hormone agonists;
(30) dehydroepiandrosterone;
(31) DGAT1 (diacylglycerol acyltransferase 1) inhibitors;
(32) DGAT2 (diacylglycerol acyltransferase 2) inhibitors;
(33) dicarboxylate transporter inhibitors;
(34) diethylpropion hydrochloride (Tenuate);
(35) dipeptidyl peptidase IV (DP-IV) inhibitors, such as isoleucine thiazolidide, valine pyrrolidide, NVP-DPP728, LAF237, P9301, TSL 225, TMC-2A/2B/2C, FE 999011, P9310/K364, VIP 0177, SDZ 274-444 and the compounds disclosed in PCT publication Nos. WO02/083128, WO02/062764, WO03/000180, WO03/000181, WO03/000250, WO03/002530, WP03/002531, WO03/002553, WO03/002593, WO03/004498, WO03/004496, WO03/017936, WO03/024942, WO03/024965, WO03/033524, WO03/037327 and EP1258476;
(36) ephedra;
(37) exendin-4 (an inhibitor of glp-1)
(38) FAS (fatty acid synthase) inhibitors, such as Cerulenin and C75;
(39) fat resorption inhibitors such as those in WO03/053451, and the like;
(40) fatty acid transporter inhibitors;
(41) fiber (psyllium, plantago, guar fiber);
(42) galanin antagonists;
(43) galega (Goat's Rue, French Lilac);
(44) garcinia cambogia;
(45) germander (teucrium chamaedrys);
(46) ghrelin antagonists, such as those disclosed in PCT Application Nos. WO 01/87335, and WO 02/08250;
(47) GLP-1 (glucagon-like peptide 1) agonists (e.g. exendin-4);
(48) glp-1 (glucagon-like peptide-1);
(49) glucocorticoid antagonists;
(50) glucose transporter inhibitors;
(51) growth hormone secretagogue receptor agonists/antagonists, such as NN703, hexarelin, MK-0677, SM-130686, CP-424,391, L-692,429 and L-163,255, and such as those disclosed in U.S. Pat. No. 6,358,951, U.S. Patent Application Nos. 2002/049196 and 2002/022637, and PCT Application Nos. WO 01/56592 and WO 02/32888;
(52) growth hormone secretagogues, such as those disclosed and specifically described in U.S. Pat. No. 5,536,716;
(53) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in PCT publication No. WO02/15905 and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO03/024928 and WO03/024929;
(54) interleukin-6 (IL-6) and modulators thereof, as in WO03/057237, and the like;
(55) L-camitine;
(56) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520;
(57) leptin, including recombinant human leptin (PEG-OB, Hoffman La Roche) and recombinant methionyl human leptin (Amgen);
(58) lipase inhibitors, such as tetrahydrolipstatin (orlistat/Xenical®), Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in PCT publication No. WO01/77094, and U.S. Pat. Nos. 4,598,089, 4,452,813, 5,512,565, 5,391,571, 5,602,151, 4,405,644, 4,189,438, and 4,242,453;
(59) lipid metabolism modulators such as maslinic acid, erythrodiol, ursolic acid uvaol, betulinic acid, betulin, and the like and compounds disclosed in WO03/011267;
(60) Mc3r (melanocortin 3 receptor) agonists;
(61) Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410;
(62) Mc5r (melanocortin 5 receptor) modulators, such as those disclosed in WO97/19952, WO00/15826, WO00/15790, US 20030092041;
(63) MCH2R (melanin concentrating hormone 2R) agonist/antagonists;
(64) melanin concentrating hormone antagonists;
(65) melanin-concentrating hormone 1 receptor (MCHR) antagonists, such as T-226296 (Takeda), SNP-7941 (Synaptic), and those disclosed WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480 and Japanese Patent Application Nos. JP 13226269, and JP1437059;
(66) melanocortin agonists, such as Melanotan II or those described in WO 99/64002 and WO 00/74679;
(67) Metformin (GLUCOPHAGE®);
(68) mGluR5 modulators such as those disclosed in WO03/029210, WO03/047581, WO03/048137, WO03/051315, WO03/051833, WO03/053922, WO03/059904, and the like;
(69) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO01/27068, and WO01/62341;
(70) NE (norepinephrine) transport inhibitors, such as GW 320659, despiramine, talsupram, and nomifensine;
(71) nomame herba;
(72) non-selective serotonin/norepinephrine transport inhibitors, such as sibutramine or fenfluramine;
(73) NPY 1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528;
(74) NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683, European Patent Nos. EP-01010691, and EP-01044970 and PCT Publication Nos. WO97/19682, WO97/20820, WO97/20821, WO97/20822, WO97/20823, WO98/27063, WO00/107409, WO00/185714, WO00/185730, WO00/64880, WO00/68197, WO00/69849, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, W002/22592, WO02/48152, WO02/49648, WO02/051806, WO02/094789, W003/009845, WO03/014083, WO03/022849, WO03/028726 and Norman et al., J. Med. Chem. 43:4288-4312 (2000);
(75) opioid antagonists, such as nalmefene (REVEX®), 3-methoxynaltrexone, naloxone, and naltrexone and those disclosed in WO00/21509;
(76) orexin antagonists, such as SB-334867-A and those disclosed in PCT publication Nos. WO01/96302, WO01/68609, WO02/44172, WO02/51232, WO02/51838, WO02/089800, WO02/090355, WO03/023561, WO03/032991, and WO03/037847;
(77) PDE (phosphodiesterase) inhibitors including but not limited to those disclosed in U.S. Pat. No. 6,331,543 and cilomilast, cilostamide, denbufyllene piclamilast, filaminast, milrinone, pentoxifylline, roflumilast, rolipram, sildenafil(Viagra®), tadalafil(Cialis®), theophylline, vardenafil(Levitra®), zaprinast, and zardaverine;
(78) peptide YY and fragments and variants thereof (e.g. YY3-36 (PYY3-36)(N. Engl. J. Med. 349:941,2003; IKPEAPGE DASPEELNRY YASLRHYLNL VTRQRY (SEQ ID NO:XXX)) and PYY agonists such as those disclosed in WO03/026591;
(79) phendimetrazine;
(80) phentermine,
(81) phosphate transporter inhibitors;
(82) phosphodiesterase-3B (PDE3B) inhibitors;
(83) phytopharm compound 57 (CP 644,673);
(84) pyruvate;
(85) SCD-1 (stearoyl-CoA desaturase-1) inhibitors;
(86) serotonin reuptake inhibitors, such as dexfenfluramine, fluoxetine, and those in U.S. Pat. No. 6,365,633, and WO01/27060, and WO01/162341;
(87) T71 (Tularik; Inc.; Boulder Co.);
(88) thyroid hormone β agonists, such as KB-2611 (KaroBioBMS), and those disclosed in WO02/15845 and Japanese Patent Application No. JP 2000256190;
(89) Topiramate (TOPIMAX®);
(90) transcription factor modulators such as those disclosed in WO03/026576;
(91) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propeny-1]benzoic acid (TTNPB), retinoic acid, and those disclosed in PCT Patent Application No. WO 99/00123;
(92) β3 (beta adrenergic receptor 3) agonists, such as AD9677/TAK677 (Dainippon/Takeda), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), and SR 591 19A, and those disclosed in U.S. Pat. No. 5,705,515, U.S. Pat. No. 5,451,677 and PCT publication Nos. WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881;
(93) β-hydroxy steroid dehydrogenase-1 inhibitors (β-HSD-1) and
(94) β-hydroxy-β-methylbutyrate.

### Methods of Treatment

Chymotrypsin inhibitors can be used to treat conditions in which it would be useful to potentiate the action of guanylin whether the guanylin is endogenous or is administered exogenously. Guanylin is a GC-C receptor agonist as are uroguanylin, lymphoguanylin, renoguanylin, and E. coli heat stable ST peptide. A number of disorders can possibly be treated with GC-C receptor agonists. Because guanylin is a GC-C receptor agonist, these disorders can possibly be treated with guanylin or a biologically active variant or fragment thereof.

The inhibitors of the invention can be used alone or in combination therapy for the treatment or prevention of congestive heart failure and benign prostatic hyperplasia. Such agents can be used in combination with natriuretic peptides (e.g., atrial natriuretic peptide, brain natriuretic peptide or C-type natriuretic peptide), a diuretic, or an inhibitor of angiotensin converting enzyme.

The inhibitors of the invention can be used alone or in combination therapy for the treatment or prevention of obesity-related disorders (e.g. disorders that are associated with, caused by, or result from obesity). Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrhythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Tumer's syndrome, and other pathological conditions showing reduced metabolic activity or a decrease in resting energy expenditure as a percentage of total fat-free mass, e.g., children with acute lymphoblastic leukemia. The compounds of the invention may be used to reduce or control body weight (or fat) or to prevent and/or treat obesity or other appetite related disorders related to the excess consumption of food, ethanol and other appetizing substances. The compounds may be used to modulate lipid metabolism, reduce body fat (e.g. via increasing fat utilization) or reduce (or suppress) appetite (e.g. via inducing satiety). Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastroesophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The agents of the present invention are also useful for reducing the risk of secondary outcomes of obesity, such as reducing the risk of left ventricular hypertrophy.

The inhibitors of the invention can be used alone or in combination therapy for the treatment or prevention of gastrointestinal related disorders including: chronic intestinal pseudo-obstruction (Ogilvie's syndrome), colonic pseudoobstruction, Crohn's disease, dyspepsia (including functional dyspepsia or nonulcer dyspepsia), functional bowel disorder, functional gastrointestinal disorders, functional heartburn, gastroesophageal reflux disease (GERD), gastrointestinal motility disorders, gastroparesis (e.g. idopathic gastroparesis), hypertrophic pyloric stenosis, Inflammatory bowel disease, irritable bowel syndrome (IBS), post-operative ileus, and ulcerative colitis. The inhibitors of the invention can be used alone or in combination therapy to patient suffering from or susceptible to GI disorders relating to damage to the GI tract stemming from impact or surgical intervention. The inhibitors of the invention can be used alone or in combination therapy to patients at risk for or having particular diseases are associated with hypomotility or stasis in the GI tract. For example, diabetic neuropathy, anorexia nervosa, and achlorhydria are frequently accompanied by gastric hypomotility. Damage to the GI tract following surgical intervention, for instance, can result in substantial gastric stasis. The inhibitors of the invention can be administered alone or in combination therapy to patients susceptible to or having a GI disorder associated with diabetes (e.g. diabetic gastropathy). The inhibitors of the invention can be used alone or in combination therapy to prevent and/or treat GI disorders characterized by at least one of nausea, vomiting, heartburn, postprandial discomfort, diarrhea, constipation, indigestion or related symptoms. The inhibitors of the invention can be used alone or in combination therapy to prevent and/or treat GI disorders associated with at least one of diabetes, anorexia nervosa, bulimia, achlorhydria, achalasia, anal fissure, irritable bowel syndrome, intestinal pseudoobstruction, scleroderma and gastrointestinal damage

The inhibitors can be used to prevent and/or treat constipation (e.g. constipation associated with use of a therapeutic agent; constipation associated with a neuropathic, metabolic or endocrine disorder (including autonomic neuropathy, Chagas disease, cystic fibrosis, diabetes mellitus, Hirschsprung disease, hyperthyroidism, hypocalcaemia, hypothyroidism, Multiple Sclerosis, neurofibromatosis, Parkinson's disease, and spinal cord lesions); post-surgical constipation (postoperative ileus); constipation associated with a gastrointestinal disorder; idiopathic constipation (functional constipation or slow transit constipation); constipation associated with the use of analgesic drugs (e.g. opioid induced constipation); constipation associated with the use of other agents (e.g. antihypertensives, anticonvulsants, antidepressants, antispasmodics and antipsychotics); megacolon; and chronic constipation).

The inhibitors can be used to prevent and/or treat gastrointestinal immotility, or other conditions calling for laxative or stool softener therapy. Gastrointestinal immotility can include constipation, and also includes delayed oral cecal transit time, irregular Taxation, and other related gastrointestinal motility disfunction including impaction. Impaction is a condition where a large mass of dry, hard stool develops in the rectum, often due to chronic constipation. This mass may be so hard that it cannot be excreted. The subjects affected byconstipation or gastrointestinal immotility can be refractory to laxative therapy and/or stool softener therapy.

The agents of the invention can be used for the treatment or prevention of cancer, pre-cancerous growths, or metastatic growths. For example, they can be used for the prevention or treatment of: colorectal/local metastasized colorectal cancer, gastrointestinal tract cancer, lung cancer, cancer or pre-cancerous growths or metastatic growths of epithelial cells, polyps, breast, colorectal, lung, ovarian, pancreatic, prostatic, renal, stomach, bladder, liver, esophageal and testicular carcinoma, carcinoma (e.g., basal cell, basosquamous, Brown-Pearce, ductal carcinoma, Ehrlich tumor, Krebs, Merkel cell, small or non-small cell lung, oat cell, papillary, bronchiolar, squamous cell, transitional cell, Walker), leukemia (e.g., B-cell, T-cell, HTLV, acute or chronic lymphocytic, mast cell, myeloid), histiocytonia, histiocytosis, Hodgkin's disease, non-Hodgkin's lymphoma, plasmacytoma, reticuloendotheliosis, adenoma, adeno-carcinoma, adenofibroma, adenolymphoma, ameloblastoma, angiokeratoma, angiolymphoid hyperplasia with eosinophilia, sclerosing angioma, angiomatosis, apudoma, branchionia, malignant carcinoid syndrome, carcinoid heart disease, carcinosarcoma, cementoma, cholangioma, cholesteatoma, chondrosarcoma, chondroblastoma, chondrosarcoma, chordoma, choristoma, craniopharyngioma, chrondroma, cylindroma, cystadenocarcinoma, cystadenoma, cystosarconia phyllodes, dysgenninoma, ependymoma, Ewing sarcoma, fibroma, fibrosarcoma, giant cell tumor, ganglioneuroma, glioblastoma, glomangioma, granulosa cell tumor, gynandroblastoma, hamartoma, hemangioendothelioma, hemangioma, hemangio-pericytoma, hemangiosarcoma, hepatoma, islet cell tumor, Kaposi sarcoma, leiomyoma, leiomyosarcoma, leukosarcoma, Leydig cell tumor, lipoma, liposarcoma, lymphaugioma, lymphangiomyoma, lymphangiosarcoma, medulloblastoma, meningioma, mesenchymoma, mesonephroma, mesothelioma, myoblastoma, myoma, myosarcoma, myxoma, myxosarcoma, neurilemmoma, neuroma, neuroblastoma, neuroepithelioma, neurofibroma, neurofibromatosis, odontoma, osteoma, osteosarcoma, papilloma, paraganglioma, paraganglionia. nonchroinaffin, pinealoma, rhabdomyoma, rhabdomyosarcoma, Sertoli cell tumor, teratoma, theca cell tumor, and other diseases in which cells have become dysplastic, immortalized, or transformed.

The inhibitors of the invention can be used for the treatment or prevention of: Familial Adenomatous Polyposis (FAP) (autosomal dominant syndrome) that precedes colon cancer, hereditary nonpolyposis colorectal cancer (HNPCC), and inherited autosomal dominant syndrome.

For treatment or prevention of cancer, pre-cancerous growths and metastatic growths, the inhibitors can be used in combination therapy with radiation or chemotherapeutic agents, an inhibitor of a cGMP-dependent phosphodiesterase or a selective cyclooxygenase-2 inhibitor. (a number of selective cyclooxygenase-2 inhibitors are described in WO02062369, hereby incorporated by reference).

The inhibitors can be for treatment or prevention of inflammation. Thus, they can be used alone or in combination with inhibitor of cGMP-dependent phosphodiesterase or a selective cyclooxygenase-2 inhibitor for treatment of: organ inflammation, IBD (e.g, Crohn's disease, ulcerative colitis), asthma, nephritis, hepatitis, pancreatitis, bronchitis, cystic fibrosis, ischemic bowel diseases, intestinal inflammations/allergies, coeliac disease, proctitis, eosnophilic gastroenteritis, mastocytosis, and other inflammatory disorders.

The inhibitors can also be used to treat or prevent insulin-related disorders, for example: II diabetes mellitus, hyperglycemia, obesity, disorders associated with disturbances in glucose or electrolyte transport and insulin secretion in cells, or endocrine disorders. They can be also used in insulin resistance treatment and post-surgical and non-post surgery decrease in insulin responsiveness.

The inhibitors can be used to prevent and/or treat pulmonary and respiratory related disorders, including, inhalation, ventilation and mucus secretion disorders, pulmonary hypertension, chronic obstruction of vessels and airways, and irreversible obstructions of vessels and bronchi. One may administer an agent of the invention for treating bronchospasm, for inducing bronchodilation, for treating chronic obstructive pulmonary disease (including chronic bronchitis with normal airflow), for treating asthma (including bronchial asthma, intrinsic asthma, extrinsic asthma, chronic or inveterate asthma (e.g. late asthma and airways hyper-responsiveness), dust-induced asthma, allergen-induced asthma, viral-induced asthma, cold-induced asthma, pollution-induced asthma and exercise-induced asthma) and for treating rhinitis (including acute, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis). This invention may also be useful in the treatment of dry eye disease and chronic sinusitis. The agents of the invention may also be used to prevent and/or treat disorders characterized by acute pulmonary vasoconstriction such as may result from pneumonia, traumatic injury, aspiration or inhalation injury, fat embolism in the lung, acidosis inflammation of the lung, adult respiratory distress syndrome, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, herapin-protamine reactions, sepsis, status asthmaticus or hypoxia (including iatrogenic hypoxia) and other forms of reversible pulmonary vasoconstriction. Such pulmonary disorders also are also characterized by inflammation of the lung including those associated with the migration into the lung of nonresident cell types including the various leucocyte subclasses. Also included in the respiratory disorders contemplated are: bullous disease, cough, chronic cough associated with inflammation or iatrogenic induced, airway constriction, pigeon fancier's disease, eosinophilic bronchitis, asthmatic bronchitis, chronic bronchitis with airway obstruction (chronic obstructive bronchitis), eosinophilic lung disease, emphysema, farmer's lung, allergic eye diseases (including allergic conjunctivitis, vernal conjunctivitis, vernal keratoconjunctivitis, and giant papillary conjunctivitis), idiopathic pulmonary fibrosis, cystic fibrosis, diffuse pan bronchiolitis and other diseases which are characterized by inflammation of the lung and/or excess mucosal secretion. Other physiological events which are contemplated to be prevented, treated or controlled include platelet activation in the lung, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), chronic obstructive pulmonary disease (COPD)(such as irreversible COPD), chronic sinusitis, fibroid lung, hypersensitivity lung diseases, hypersensitivity pneumonitis, idiopathic interstitial pneumonia, nasal congestion, nasal polyposis, and otitis media.

The inhibitors can be used in combination therapy with a phosphodiesterase inhibitor. (examples of such inhibitors can be found in U.S. Pat. No. 6,333,354, hereby incorporated by reference).

The inhibitors can be used alone or in combitherapy to prevent or treat: retinopathy, nephropathy, diabetic angiopathy, and edema formation The inhibitors can be used alone or in combitherapy to prevent or treat neurological disorders, for example, headache, migraines, anxiety, stress, cognitive disorders, cerebral ischemia, brain trauma, movement disorders, aggression, psychosis, seizures, panic attacks, hysteria, sleep disorders, depression, schizoaffective disorders, sleep apnea, attention deficit syndromes, memory loss, dementia, memory and learning disorders as discussed in Moncada and Higgs 1995 FASEB J. 9:1319-1330; Severina 1998 Biochemistry 63:794; Lee et al. 2000 PNAS 97: 10763-10768; Hobbs 1997 TIPS 18:484-491; Murad 1994 Adv. Pharmacol. 26:1-335; and Denninger et al. 1999 Biochim. Biophys. Acta 1411:334-350 and narcolepsy. They may also be used as a sedative.

The inhibitors and detectably labeled inhibitors can be used as markers to identify, detect, stage, or diagnosis diseases and conditions of small intestine, including: Crohn's disease, colitis, inflammatory bowel disease, tumors, benign tumors, such as benign stromal tumors, adenoma, angioma, adenomatous (pedunculated and sessile) polyps, malignant, carcinoid tumors, endocrine cell tumors, lymphoma, adenocarcinoma, foregut, midgut, and hindgut carcinoma, gastroinstestinal stromal tumor (GIST), such as leiomyorna, cellular leiomyoma, leiomyoblastoma, and leiomyosarcoma, gastrointestinal autonomic nerve tumor, malabsorption syndromes, celiac diseases, diverticulosis, Meckel's diverticulum, colonic diverticula, megacolon, Hirschsprung's disease, irritable bowel syndrome, mesenteric ischemia, ischemic colitis, colorectal cancer, colonic polyposis, polyp syndrome, intestinal adenocarcinoma, Liddle syndrome, Brody myopathy, infantile convulsions, and choreoathetosis

The inhibitors can be used in combitherapy with a GC-C agonist (e.g. guanylin or a biologically active variant or fragment thereof) conjugated to another molecule (e.g., a diagnostic or therapeutic molecule) to target cells bearing the GC-C receptor, e.g., cystic fibrosis lesions and specific cells lining the intestinal tract. Thus, they can be used to target radioactive moieties or therapeutic moieties to the intestine to aid in imaging and diagnosing or treating colorectal/metastasized or local colorectal cancer and to deliver normal copies of the p53 tumor suppressor gene to the intestinal tract. The inhibitors can also be used to increase the number of GC-C molecules on the surface of a cell. In some embodiments the cell is a metastasized colorectal cancer cell. In one embodiment the inhibitor is used administered in combitherapy with a GC-C agonist which is therapeutically conjugated to a second agent. In certain embodiments, the second agent can be radioactive or radiostable. In certain embodiments the second agent can be selected from the group consisting of a compound that causes cell death, a compound that inhibits cell division, a compound that induces cell differentiation, a chemotherapeutic, a toxin and a radiosensitizing agent. In certain embodiments the second agent can be selected from the group consisting of: methotrexate, doxorubicin, daunorubicin, cytosinarabinoside, etoposide, 5-4 fluorouracil, melphalan, chlorambucil, cis-platin, vindesine, mitomycin, bleomycin, purothionin, macromomycin, 1,4-benzoquinone derivatives, trenimon, ricin, ricin A chain, Pseudomonas exotoxin, diphtheria toxin, Clostridium perfringens phospholipase C, bovine pancreatic ribonuclease, pokeweed antiviral protein, abrin, abrin A chain, cobra venom factor, gelonin, saporin, modeccin, viscumin, volkensin, nitroimidazole, metronidazole and misonidazole.

The inhibitors can be used alone or in combination therapy to prevent and/or treat inner ear disorders, e.g., to prevent and/or treat Meniere's disease, including symptoms of the disease such as vertigo, hearing loss, tinnitus, sensation of fullness in the ear, and to maintain fluid homeostasis in the inner ear.

The inhibitors can be used alone or in combination therapy to prevent and/or treat disorders associated with fluid and sodium retention, e.g., diseases of the electrolyte-water/electrolyte transport system within the kidney, gut and urogenital system, congestive heart failure, hypertension, hypotension, salt dependent forms of high blood pressure, hepatic edema, and liver cirrhosis. In addition they can be used to facilitate diuresis or control intestinal fluid. The inhibitors can also be used to treat disorders where there is abnormal proliferation of epithelial cells within the kidney (e.g. as in the case of renal cancer).

The inhibitors can be used alone or in combination therapy to prevent and/or treat kidney disease. “Kidney disease” includes renal failure (including acute renal failure), renal insufficiency, nephrotic edema, glomerulonephritis, pyelonephritis, kidney failure, chronic renal failure, nephritis, nephrosis, azotemia, uremia, immune renal disease, acute nephritic syndrome, rapidly progressive nephritic syndrome, nephrotic syndrome, Berger's Disease, chronic nephritic/proteinuric syndrome, tubulointerstital disease, nephrotoxic disorders, renal infarction, atheroembolic renal disease, renal cortical necrosis, malignant nephroangiosclerosis, renal vein thrombosis, renal tubular acidosis, renal glucosuria, nephrogenic diabetes insipidus, Bartter's Syndrome, Liddle's Syndrome, polycystic kidney disease, medullary cystic disease, medullary sponge kidney, hereditary nephritis, and nail-patella syndrome, along with any disease or disorder that relates to the renal system and related disorders, as well as symptoms indicative of, or related to, renal or kidney disease and related disorders.

The inhibitors can be used alone or in combination therapy to prevent or treat polycystic kidney disease. Polycystic kidney disease” “PKD” (also called “polycystic renal disease”) refers to a group of disorders characterized by a large number of cysts distributed throughout dramatically enlarged kidneys. The resultant cyst development leads to impairment of kidney function and can eventually cause kidney failure. “PKD” specifically includes autosomal dominant polycystic kidney disease (ADPKD) and recessive autosomal recessive polycystic kidney disease (ARPKD), in all stages of development, regardless of the underlying cause.

The inhibitors can be used alone or in combination therapy to prevent and/or treat disorders associated with bicarbonate secretion, e.g., Cystic Fibrosis.

The inhibitors can be used alone or in combination therapy to prevent and/or treat disorders associated with bile secretion. In addition, they can be used to facilitate or control chloride and bile fluid secretion in the gall bladder.

The inhibitors can be used alone or in combination therapy to prevent and/or treat disorders associated with liver cell regeneration. This may include administration of the inhibitors to liver transplant recipients and to patients with drug or alcohol induced-liver damage. Furthermore, the inhibitors may be useful to treat liver damage as in the case of viral mediated hepatitis. The inhibitors may be used alone or in combination to prevent and/or treat liver abscess, liver cancer (either primary or metastatic), cirrhosis (such as cirrhosis caused by the alcohol consumption or primary biliary cirrhosis), amebic liver abscess, autoimmune hepatitis, biliary atresia, coccidioidomycosis disseminated, δ agent (hepatitis δ), hemochromatosis, hepatitis a, hepatitis b, hepatitis c, or any other acute, subacute, fulminant or chronic hepatitis of viral, metabolic or toxic etiology, hepatocellular carcinoma, pyogenic liver abscess, Reye's syndrome, sclerosing cholangitis, Wilson's disease, drug induced hepatotoxicity, or fulminant or acute liver failure. The inhibitors may be used in stimulating hepatic regeneration after surgical hepatectomy.

The inhibitors can be used alone or in combination therapy to prevent and/or treat myocardial infraction, diastolic dysfunction, angina pectoris, stable, unstable and variant (Prinzmetal) angina, atherosclerosis, thrombosis, endothelial dysfunction, cardiac edema, stroke, conditions of reduced blood vessel patency, e.g., postpercutaneous transluminal coronary angioplasty (post-PTCA) and peripheral vascular disease,

The inhibitors can be used alone or in combination therapy to prevent and/or treat glaucoma.

The inhibitors can be used alone or in combination therapy to prevent and/or treat immunodeficiency.

The inhibitors can be used alone or in combination therapy to prevent and/or treat bladder outlet obstruction and incontinence.

The inhibitors can be used alone or in combination therapy to prevent and/or treat male (e.g. erectile dysfunction) or female sexual dysfunction, premature labor, and dysmenorrhoea.

The inhibitors can be used alone or in combination therapy to prevent and/or treat osteopenia disorders (bone loss disorders). “Bone loss disorders” include conditions and diseases wherein the inhibition of bone loss and/or the promotion of bone formation is desirable. Among such conditions and diseases are osteoporosis, osteomyelitis, Paget's disease (osteitis deformans), periodontitis, hypercalcemia, osteonecrosis, osteosarcoma, osteolyic metastases, familial expansile osteolysis, prosthetic loosening, periprostetic osteolysis, bone loss attendant rheumatoid arthritis, and cleiodocranial dysplasia (CCD). Osteoporosis includes primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Rile-Day syndrome) and osteoporosis due to immobilization of extremitiesosteomyelitis, or an infectious lesion in bone leading to bone loss. The inhibitors can be used alone or in combination therapy to stimulating bone regeneration. The bone regeneration may be following reconstruction of bone defects in cranio-maxillofacial surgery, or following an implant into bone, for example a dental implant, bone supporting implant, or prosthesis. The bone regeneration may also be following a bone fracture.

### OTHER EMBODIMENTS

### HIV Protease Inhibitors and Reduction in Guanylin Activity

Diarrhea is a common complication in HIV patients. It appears that diarrhea is particularly common in patients treated with protease inhibitors. Prolonged diarrhea impacts quality of life and can contribute to weight loss, malnutrition, immunosuppression, poor drug absorption, non-compliance with therapy and mortality. Certain HIV protease inhibitors also inhibit chymotrypsin. Thus, treatment with HIV protease inhibitors may reduce chymotrypsin activity in the gastrointestinal tract and this may lead to increased levels of active guanylin. Since guanylin promotes intestinal motility, this increase in active guanylin can lead to diarrhea and other gastrointestinal disorders. Thus, the invention features methods for treating or preventing diarrhea and/or other gastrointestinal disorders in patients, particularly HIV patients and others being treated with a protease inhibitor that inhibits chymotrypsin, by administering to the patient chymotrypsin. The administered chymotrypsin should reduce the level of guanylin.

The gastrointestinal side-effects of HIV protease inhibitors can be mitigated by modifying the inhibitors to have reduced chymotrypsin inhibition activity. Thus, the invention features a method for identifying protease inhibitors, e.g., HIV protease inhibitors, having reduced chymotrypsin inhibition activity and thus reduced gastrointestinal side effects. The methods entail testing a known or candidate protease inhibitor for its ability to inhibit chymotrypsin and selecting a known or candidate protease inhibitor having reduced chymotrypsin inhibition and retaining the ability to inhibit HIV protease. The ability of a compound to inhibit HIV protease and the ability of a compound to inhibit chymotrypsin can be assessed using standard in vitro assays including the guanylin degradation assay described herein.

The invention also feature compounds that are analogues of an HIV protease inhibitor and have reduced chymotrypsin inhibition activity while retaining the ability to inhibit HIV protease.

As noted above, certain HIV protease inhibitors may inhibit chymotrypsin and may undesirably potentiate the activity of guanylin. Among these inhibitors are: ritonavir (André et al. 1998 Proc Natl Acad Sci USA 95:13120-4 and Schmidtke et al. 1999 J Biol Chem 274:35734-40), saquinavir (Hosseini et al. 2001 J Neuroimmunol. 118:233-44) and indinavir/indavir (Piccinini et al. 2002 AIDS 16:693-700). These HIV protease inhibitors and others have the ability to inhibit chymotrypsin and this may lead to gastrointestinal side-effects. To reduce these side-effects, a patient being treated with such an inhibitor can also be treated with uroguanylin or guanylin or an analogue or variant of uroguanylin or guanylin.

An HIV protease inhibitor variant having reduced chymotrypsin inhibition will be less likely to potentiate the action of guanylin. Thus, such inhibitors are less likely to be associated with gastrointestinal side-effects. HIV protease inhibitors such as those depicted below can be modified to reduce chymotrypsin inhibition. The modification can include replacing a hydroxyl group with a phosphonate group or with an ester (e.g., cycloalkyllester, arylalkylester, aralkylester, heteroaralkylester). Thus, any hydroxyl group in the compounds below can be replaced by —OR wherein R is a C₁ to C₁₂ alkyl group or a C₆-C₁₈ where alkyl refers to a hydrocarbon chain that may be a straight chain or branched chain, containing the indicated number of carbon atoms. For example, C₁-C₁₂ alkyl indicates that the group may have from 1 to 12 (inclusive) carbon atoms in it (i.e., 1, 2, 3, 4, 5, 6, 7, 8, 9, 10, 11, 12). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups. The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl. The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbomyl, and adamantyl. Saquinavir (CAS Registry No. 127779-20-8)

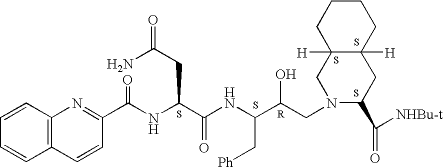

Indavir (CAS Registry No 150378-17-9)

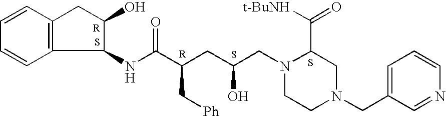

Norvir (CAS Registry No. 155213-67-5)

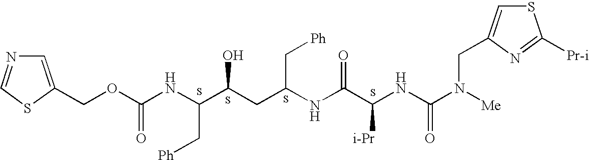

Nelfinavir (CAS Registry No. 159989-64-7)

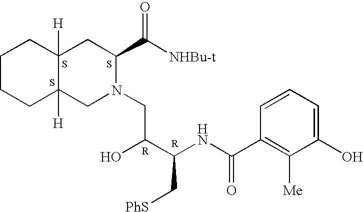

Amprinavir (CAS Registry No. 161814-49-9)

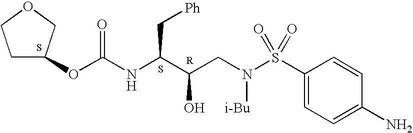

Atazanivir (CAS Registry No. 198904-31-3)

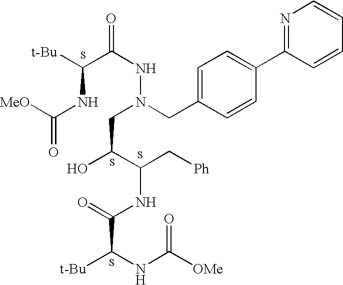

Lopinavir (CAS Registry No. 369372-47-4)

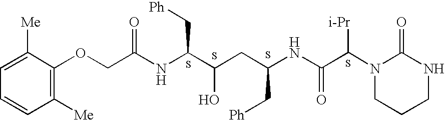

It has been suggested that the chymotrypsin like activity of the 20S proteasome is inhibited by HIV protease inhibitors. Thus, it can be desirable to assess chymotrypsin inhibitors and HIV protease inhibitors using an assay that measures the chymotrypsin-like activity of the proteasome. One such assay is described by Elliott et al. (Methods in Molecular Medicine 85:163-172, 2003). This assay can be used to accurately determine the level of proteasome activity in rodent blood samples. The assay entails measuring proteasome activity at either or both of the two proteolytic sites (chymotryptic and tryptic) within the 20S core of the proteasome and determining degree of inhibition conferred by a test agent.

1. A method for increasing GC-C Receptor activity, the method comprising administering a composition comprising a chymotrypsin inhibitor.

2-3. (canceled)

4. A method for increasing intestinal motility, the method comprising administering a composition comprising a chymotrypsin inhibitor.

5-7. (canceled)

8. The method of claim 20 wherein the gastrointestinal disorder is selected from the group consisting of: constipation, a gastrointestinal motility disorder, irritable bowel syndrome, a functional gastrointestinal disorder, gastroesophageal reflux disease, functional heartburn, dyspepsia, functional dyspepsia, nonulcer dyspepsia, gastroparesis, chronic intestinal pseudo-obstruction, and colonic pseudo-obstruction.

9-11. (canceled)

12. The method of any of claim 1 further comprising administering guanylin or a biologically active variant or fragment thereof.

13. A pharmaceutical composition comprising a chymotrypsin inhibitor and a pharmaceutically acceptable carrier.

14. The composition of claim 10 further comprising guanylin or a biologically active variant or fragment thereof.

15. The composition of claim 14 further comprising a GC-C Receptor agonist.

16. The method of claim 1 further comprising administering a GC-C Receptor agonist.

17. (canceled)

18. The method of claim 1 wherein the disorder is congestive heart failure.

19. The method of claim 1 wherein the disorder is prostatic hyperplasia.

20. The method of claim 1 wherein the disorder is a gastrointestinal disorder.

21. The method of claim 1 wherein the disorder is obesity.
